(19) United States
(12) Patent Application Publication
(10) Pub. No.: US 2023/0108454 A1
(43) Pub. Date: Apr. 6, 2023

(54) SYSTEM AND METHODS FOR CODE ANALYSIS AND DATA STORAGE WITH CRYPTOGRAPHY

(71) Applicant: BRELLA INSURANCE, INC., Claymont, DE (US)

(72) Inventors: Veer Gidwaney, Hoboken, NJ (US); Abigail Albright, Boston, MA (US); Amanda Turcotte, Montclair, NJ (US); Michael Zarrillo, Charlotte, NC (US)

(21) Appl. No.: 17/853,535

(22) Filed: Jun. 29, 2022

Related U.S. Application Data
(60) Provisional application No. 63/219,661, filed on Jul. 8, 2021.

(51) Int. Cl. G06Q 40/08

(52) U.S. Cl. CPC G06Q 40/08

(57) ABSTRACT

The disclosure is directed to systems and methods for the transfer of documentation over secure networks. In some embodiments, the system includes cryptography that enables network security while displaying graphical user interfaces and receiving inputs into the system. In some embodiments, the inputs include one or more forms which the system is configured to determine codes. In some embodiments, the inputs include information pertaining to confidential data. In some embodiments, the system includes modules that enable users to enroll in programs and receive payments over secure networks relating to the programs.

### CROSS-REFERENCE TO RELATED APPLICATION

This application claims the benefit and priority of U.S. Provisional Application No. 63/219,661, filed Jul. 8, 2021, which is incorporated herein by reference in its entirety.

### BACKGROUND

The primary purpose of a supplemental health plan is to reduce unexpected out-of-pocket medical costs. This is a critical need today as the cost of employer-sponsored health coverage and associated out of pocket cost-sharing has become increasingly burdensome for employees. The average employee pays $5,588 in premiums for family health coverage that comes with an average deductible of $3,655. Yet, a recent study found that 60% of Americans would struggle to pay a $1,000 unexpected expense (Source: Bankrate Financial Security Index January 2020). Conventional methods of providing supplemental health insurance and submitting, adjudicating and processing claims were designed decades ago and have not evolved to address today's challenges.

In addition, the narrow scope of conventional supplemental coverage leads directly to low utilization rates. Misalignment of benefits with the employee's actual financial responsibility leaves many people financially exposed, and complex rules and claims requirements cause employees to miss opportunities to obtain the benefits they need to help ease the financial burden of a medical event. Poor strategic ties to overall health plan objectives have caused today's most common supplemental health insurance options to demonstrate poor product/market fit. Furthermore, conventional methods of obtaining and evaluating claim information is a burden on network and computer resources, and often does not include secure networks or cryptography techniques during collection. Therefore, there is a need for an intelligent insurance design, pricing, automated administration, and claim submission system to overcome the limitations of conventional systems to provide comprehensive coverage which better fits current and future market needs.

### SUMMARY

Some embodiments provide an appointment application and an associated appointment application webform flow. In some embodiments, an appointment webform is automatically generated by the system per agency. Some embodiments of the system automatically generate a version for individual producers. This appointment webform flow may supplement or replace current processes used to manage appointments according to some embodiments. In some embodiments, the system is configured to check for a valid appointment record at the time of contract issue regardless of process. In some embodiments, DocuSign® or similar secure document transfer services are used for partner and agency contracting.

Regarding partner ongoing administration, the system enables partners to have access to a partner portal module according to some embodiments. In some embodiments, a partner portal graphical user interface (GUI) is configured to enable partners to request quotes, communicate with an underwriting module (i.e., to provide feedback, share documents, request a re-quote), view quotes, access employer details, access policy details for in force policies and contract documents, get help, manage their own users, and manage their team. In some embodiments, a book of business and/or (Brella) book management system can be accessed from the partner portal GUI. In some embodiments, the system is configured to enable access to performance dashboards (for enrollment, premium calculations, etc.) through the partner portal GUI. In some embodiments, the partner portal module is integrated into one or more modules and/or systems described herein (e.g., the core admin system, the secure processing platform/policy admin system). In some embodiments, the system is configured to enable all of the functionality that can be triggered through the partner portal module (e.g., a quote request) to be accessed from another platform, product, or process.

Some embodiments enable functionality to be turned on and off as needed due to a modular design of one or more portions of the system. As just one non-limiting example, the system is configured to provide a cobranded version of the partner portal module for partners. In some embodiments, the cobranded version that has custom branding and custom get-help messaging according to some embodiments. In some embodiments, the system is configured to enable a user to input one or more items through the partner portal GUI, receive the data from the partner portal module, and send data back to the user. One non-limiting example, according to some embodiments, is the user going to a partner portal GUI or partner portal module for book of business reporting across the full product suite, but single signing into a cobranded portal for quote requests.

Some embodiments provide employer sales functionality including quoting through underwriting module and proposal generation. In some embodiments, the quote request is initiated through the partner portal GUI. In some embodiments, the system is configured to enable a partner to request quotes for new or existing employers. In some embodiments, quote information requested by the system includes one or more employer level details (e.g., Standard Industrial Classification (“SIC”), state, census, etc.) and quote level dates, (e.g., effective date, commission, etc.), as non-limiting examples according to some embodiments. In some embodiments, the quote information then goes through the rating engine and the underwriting review process, which is performed by the automated underwriting module according to some embodiments. Based on that quote being approved in the policy admin system, in some embodiments, the proposal is automatically generated, made available within the partner portal, and a customizable notification is emailed to the partner. In some embodiments, the system is configured to enable the quote to be reviewed and/or approved by a team member before the proposal is auto generated.

In some embodiments, the system is configured generate a secure graphical user interface to enable one or more portions of the proposal to be modified by an employer and/or partner to evaluate different strategies and/or product offerings (e.g., Choice or Select, Riders or not, employer contribution scenarios, etc.) which would require re-quotes in conventional systems. In some embodiments, the system is configured to provide a modeling tool (e.g., a premium calculator tool) to aid in determining an optimum proposal modification. In some embodiments, the system is configured to generate a custom tokenized Uniform Resource Locator (“URL”) per quote. In some embodiments, the URL displays static sales and marketing content (e.g., at the top) but allows a partner and/or employer to modify inputs with regards to one or more plan offerings, benefit amounts, different employer contribution scenarios, and including or excluding riders. In some embodiments, the application is connected to the quoting and rating engine. As a non-limiting example, in some embodiments, employer contribution impacts rates, and if an employer contributes to the employee premium, a rate adjustment may be applied. If the user enters an employer contribution on the proposal user interface (“UI”), the rating engine is accessed again by the system (e.g., a secure processing platform) and automatically adjusts those rates, according to some embodiments. In some embodiments, the updated rates are shown on the proposal in real time.

In some embodiments, the system includes a proposal acceptance (application) module. In some embodiments, the proposal acceptance module is configured to automatically trigger the next piece of the contracting flow: the employer welcome and case install workflow. In some embodiments, based on the contact information provided on proposal acceptance, the system creates that employer portal and that employer portal user account and automatically sends the employer a welcome email to implement the next step of the flow.

In some embodiments, the system includes automatic implementation of one or more rules described herein. In some embodiments, for underwriting module execution, the system is configured to include limits of certain attributes and/or forced contract terms based on state regulatory requirements. In some embodiments, non-limiting examples of one or more rules include maximum benefit waiting period, maximum rate guarantee, minimum number of age bands, and/or minimum benefit amounts. In some embodiments, the automatic implementation of one or more rules is configured to impact employer implementation flows. In some embodiments, non-limiting examples of employer implementation flows include available choices and/or configuration limits based on state requirements. In some embodiments, the system is configured to reflect regulatory “forced” (required) contract term configurations in displayed shopping and/or education experiences. As a non-limiting example, Florida's child eligibility requirements, which are different from other states, are automatically implemented by the system in one or more sections described herein according to some embodiments.

Some embodiments provide fully automated employer implementation by one or more modules. In some embodiments, the employer reviews the information populated by the partner on the proposal acceptance flow. In some embodiments, the group application is automatically generated with the appropriate state specific form, plan offering and employer contribution scenarios and prepopulated with all those employer details. In some embodiments, it's then presented via an integrated secure electronic signature system (e.g., DocuSign®) through the employer portal for signature. In some embodiments, the document can be resent, or the signer can be reassigned if there needs to be a different designated signer as well.

In some embodiments, based on the completion of the group application signature, the system then updates the contract record and the policy administration system and all of the policy documents (e.g., policy and the employer version of the certificate) are automatically generated and made available simultaneously. In some embodiments, the system then moves on to open enrollment setup. In some embodiments, the open enrollment setup and remainder of the implementation is all done through an implementation form provided by an implementation module. In some embodiments, the implementation form is a web-based implementation form that is customized for each employer by the system automatically. In some embodiments, the implementation web form includes a combination of enrollment method and plan offering.

In some embodiments, the system includes different versions based on prior selections. Some non-limiting versions include one for third party benefits administration vendors, one for the enrollment platform (also referred to as Brella Enroll herein), and one for situations in which the employer is fully funding the product for their employees. In some embodiments, a provided version or configuration is then automatically generated based on the contract attributes in the policy admin system (or secure processing platform, as another non-limiting example). In some embodiments, different versions of education/recommendation are automatically provided, based on the completion of the web form. In some embodiments, changes can be made before an invoice is finalized regardless of whether Brella Enroll is employed or not.

In some embodiments, the Brella Enroll platform (enrollment platform) facilitates employee education, plan shopping, benefit recommendations, and enrollment directly on the platform, whereas the BenAdmin platform (education platform) allows employees to shop for their plan and get personalized recommendations before enrolling via an employer's enrollment system. For employers who offer the product on a non-contributory basis, the system platform enrolls employees automatically. In some embodiments, Brella Enroll includes automated enrollment campaigns.

In some embodiments, the system is configured to generate an employee shopping GUI that includes one or more different versions of a shopping experience. One such shopping experience is the Brella Enroll version, which is an enrollment tool, and the other shopping experience is a BenAdmin version, where the system takes the education, engagement, and recommendation components of the Brella Enroll experience, without the actual capturing of elections, and makes that available per employer via a URL that can be accessed and embedded within the BenAdmin platform. In some embodiments, the URL is secured by cryptography or one or more other secure data transfer systems.

In some embodiments, in the case of Brella Enroll, a user or employer will provide, via a GUI generated by the implementation module, the launch date, the open enrollment dates, upload a census, and then the system will automatically send welcome emails and open enrollment invites based on those dates and the shopping experience becomes available. In some embodiments, in the case of BenAdmin, that employer's specific links are automatically generated by the implementation module at the time the implementation form is completed and becomes available for use. In both cases, the plan design and configuration details, the rates, and any other desired information, are all called and pulled directly from the policy admin system (secure processing platform, so there is no setup, hands on keys, or configuration required, according to some embodiments.

In some embodiments, the system provides a member enrollment processing platform. In some embodiments, member processing includes receiving of the election information via the Brella Enroll platform, sending an enrollment report if the user uses a non-integrated BenAdmin, or via an inbound enrollment file. In some embodiments, the system integrates and communicates with a BenAdmin vendor via an Application Programming Interface (“API”). Once the system receives that election information, member IDs and certificates are automatically generated.

In some embodiments, once the employer is implemented and open enrollment has been setup, the employer has access to their ongoing administration portal. Here the employer can perform functions such as monitor annual enrollments, manage employees, add, terminate, trigger life events, view an employee's plan information and all policy and contract documents, view reporting and billing in one place according to some embodiments, as well as request help and access a knowledge base.

In some embodiments, the system handles billing, including calculation and invoice generation, and different frequencies as well. In some embodiments, the system generates a preview of the invoice. In some embodiments, the employer can review the preview of the invoice over a secure online connection (e.g., encrypted) before it is finalized, they can request any changes, and they can make any changes, such as adding or terminating an employee, if the employer is using a Brella Enroll platform. In some embodiments, the employer also can authorize electronic payments via their employer portal within the employer porter module as well. In some embodiments, the system is configured to automatically manage the overpayment and/or calculate adjustments. In some embodiments, the system is configured to enable a user to set up and manage automatic payments on a predetermined schedule. In some embodiments, the system is configured to automatically send one or more email notifications to the member related to their payment status (e.g., autopay reminder email six days prior to processing of payment). In some embodiments, the system is configured to bill employees directly for the product. In some embodiments, the direct billing is based on employer preferences, such as providing a partial employer contribution (e.g., split-premium). In some embodiments, the system is configured to enable the employee to provide electronic payment details at the time of enrollment. In some embodiments, all connections to billing and payment information is provided over a secure communication network employing cryptography techniques.

In some embodiments, the post enrollment experience for members focuses on employees and their covered family members. In some embodiments, both a web and a mobile app are available for members. In some embodiments, the mobile app is available on IOS® and Android® devices via the Apple® App Store® and Google Play® store. In some embodiments, both primaries and dependents can have accounts. In some embodiments, accounts can be created during the Brella Enroll shopping experience, or via a self-registration flow from a corporate site, or in the case of dependents, via an invite link from the primary member.

In some embodiments, the member portal module includes plan information and policy forms, as well as various get help options including a secure messaging inbox with a customer care team. In some embodiments where the employee is being directly billed for the product, the system is configured to provide the ability to view an invoice, set up and manage electronic payment methods, and/or to set up automatic payments. Some embodiments include the ability to set up e-payment options for claims payout. In some embodiments, Venmo®, PayPal®, and conventional bank accounts are all supported for electronic delivery of claims payments if the system approves a claim. In some embodiments, other secure methods of electronic payment that use encryption technology are contemplated.

In some embodiments, the member portal module is configured to generate one or more graphical user interfaces to enable members to file a claim via web or mobile. In some embodiments, filing a claim includes telling what happened by answering a few short questions and uploading evidence. In some embodiments, the system is flexible in terms of the evidence that it allows. In some embodiments, members then can view the status, details, and updates of any submitted claim and the determination results as well. In some embodiments, the system provides a flow in one or more graphical user interfaces for requesting additional information if needed for a claim not in good order, and that functionality includes reminders and chase emails. Some embodiments provide an internal claims adjudicator flow to review evidence and finalize the determination and integration with the member portal module and the mailing vendor for notice generation and electronic and U.S. mail delivery.

Notice/forms generation: In some embodiments, policy forms are automatically generated and compiled into a downloadable PDF packet, taking into account state specific and employer size requirements. In some embodiments, notices that communicate the status of a claim are automatically generated and account for state specific requirements. In some embodiments, the system automatically generates and sends notices relating to billing activity (e.g., error with payment, payment due).

In some embodiments, the member portal module also includes ongoing member administration, including access to policy documents, a covered condition look-up tool, claims submission, messaging with support, and a self-serve knowledge base. In some embodiments, the system also includes and supports a portability workflow. As a non-limiting example, if eligibility under ER's group policy is lost, the ability to continue their coverage under a portability trust facilitates the “moving” from certificate holder under one vs. the other, including direct billing and/or individual invoicing according to some embodiments. In some embodiments, the system provides a view for member coverage if that coverage is no longer through their employer's group policy and instead through the trust ported policy.

In some embodiments, the system is directed to secure transfer of electronic documents. In some embodiments, the system comprises one or more computers comprising one or more processors and one or more non-transitory computer readable media, the one or more non-transitory computer readable media having instructions stored thereon that when executed cause the one or more computers to implement one or more steps. In some embodiments, the one or more steps include instructions to generate, by the one or more processors, a secure processing platform. In some embodiments, the one or more steps include instructions to generate, by the one or more processors, a claims module. In some embodiments, the one or more steps include instructions to establish, by the one or more processors, a secure data connection between the processing platform and the claims module. In some embodiments, the one or more steps include instructions to send, by the one or more processors, one or more documents from the claims module to the secure processing platform. In some embodiments, the one or more steps include instructions to analyze, by the one or more processors, the one or more documents for one or more ICD codes. In some embodiments, the one or more steps include instructions to associate, by the one or more processors, the one or more ICD codes with one or more conditions.

In some embodiments, the compensation associated with the one or more conditions are sent via a data encrypted network. In some embodiments, the one or more non-transitory computer readable media have instructions stored thereon that when executed cause the one or more computers to generate, by the one or more processors, a digital marketplace module. In some embodiments, the one or more steps include instructions to send, by the one or more processors, compensation associated with the one or more conditions from the digital marketplace module. In some embodiments the one or more ICD codes are ICD-10 codes.

In some embodiments, claims module is configured to use a combination of one or more ICD codes and one or more benefit categories to determine the compensation. In some embodiments, the claims module is configured to determine if more than one benefit category is covered by analysis of one or more eligible documents. In some embodiments, the one or more steps include instructions to generate, by the one or more processors, a member portal module. In some embodiments, the one or more steps include instructions to generate, by the one or more processors, an employer portal module. In some embodiments, the secure processing platform is configured to accept one or more member inputs and one or more employer inputs from the member portal module and the employer portal module, respectively.

In some embodiments, the one or more steps include instructions to generate, by the one or more processors, a recommendation engine. In some embodiments, the one or more steps include instructions to send, by the one or more processors, the one or more member inputs and the one or more employer inputs from the secure processing platform to the recommendation engine. In some embodiments, wherein the recommendation engine is configured to apply a factor to each of the one or more member inputs and each of the one or more employer inputs. In some embodiments, the recommendation engine is configured to sum each factor. In some embodiments, the one or more steps include instructions to generate, by the one or more processors, a graphical user interface. In some embodiments, the one or more steps include instructions to display, on the graphical user interface, a partner portal. In some embodiments, the partner portal is configured to enable a user to input a quote request.

In some embodiments, the one or more steps include instructions to generate, by the one or more processors, an underwriting module. In some embodiments, the one or more steps include instructions to send, by the one or more processors, the quote request to the underwriting module. In some embodiments, the underwriting module is configured estimate risk. In some embodiments, if the risk is below a predetermined threshold, the underwriting module is configured to automatically provide a quote proportional to the risk.

### BRIEF DESCRIPTION OF THE DRAWINGS

FIG. 1 illustrates an aspect of the system that includes a single insurance product that includes three primary benefit categories: Moderate, Severe, and Catastrophic according to some embodiments.

FIG. 2 depicts two non-limiting example plan design options in the form of Choice and Select according to some embodiments.

FIG. 3 shows the system applied to an example scenario that includes 3 different employees each with a different diagnosis according to some embodiments.

FIG. 4 shows some sample age-banded rates according to some embodiments.

FIG. 5 illustrates example premium calculations according to some embodiments.

FIG. 6 portrays example plan rules and specifications according to some embodiments.

FIG. 7 shows some concepts of the claims process according to some embodiments.

FIG. 8 depicts, as a non-limiting example, points assigned to different documents according to some embodiments.

FIG. 9 depicts a claim point value for each condition group according to some embodiments.

FIG. 10 depicts example eligible claim documents for a moderate (5 pts) claim submission for pneumonia according to some embodiments.

FIG. 11 depicts example eligible claim documents for a moderate (5 pts) broken ankle claim submission according to some embodiments.

FIG. 12 depicts example eligible claim documents for a severe (8 pts) appendicitis claim submission according to some embodiments.

FIG. 13 depicts example eligible claim documents for a severe (8 pts) appendicitis claim submission according to some embodiments.

FIG. 14 depicts example eligible claim documents for a severe (8 pts) heart attack claim submission according to some embodiments.

FIG. 15 provides a summary for some embodiments of the system with 3 distinct benefit categories (Moderate, Severe, and Catastrophic) and covered conditions that correlate to International Classification of Diseases (“ICD”) 10 codes.

FIG. 16 represents an example of multiple-category determination and payout according to some embodiments.

FIG. 17 shows a flowchart for an example recommendation engine execution according to some embodiments.

FIG. 18 illustrates a non-limiting example implementation of the system according to some embodiments.

FIG. 19 illustrates a non-limiting example of computer implemented steps that include a partner portal module according to some embodiments.

FIG. 20 shows a non-limiting example of computer implemented steps that include a proposal application module according to some embodiments.

FIG. 21 depicts a non-limiting example of computer implemented steps that include a group application module according to some embodiments.

FIG. 22 shows non-limiting example of computer implemented steps that include an implementation module according to some embodiments.

FIG. 23 illustrates a non-limiting example of computer implemented steps that include an employer portal module according to some embodiments.

FIG. 24 illustrates a non-limiting example of computer implemented steps that include a member portal module according to some embodiments.

FIG. 25 shows a login including date of birth entry UI according to some embodiments.

FIG. 26 shows a personalized introduction to the employee shopping portal according to some embodiments.

FIG. 27 illustrates example questions presented in the employee shopping portal that is fed into the recommendation engine according to some embodiments.

FIG. 28 illustrates an educational portion of the employee shopping portal according to some embodiments.

FIG. 29 depicts a non-limiting recommendation engine result according to some embodiments.

FIG. 30 depicts a coverage plan generated by the recommendation engine according to some embodiments.

FIG. 31 illustrates a premium calculator portion of the system according to some embodiments.

FIG. 32 shows a claims payment election and authorization portion of the member portal according to some embodiments.

FIG. 33 illustrates a claims review UI according to some embodiments.

FIG. 34 depicts a claims information portion of the claims platform according to some embodiments.

FIG. 35 shows a claim description portion of the claims platform according to some embodiments.

FIG. 36 depicts a presentation of acceptable documents provided by the claims platform according to some embodiments.

FIG. 37 shows a user interface to upload documents for analysis by the claims platform according to some embodiments.

FIG. 38 shows a review page for the claim submission according to some embodiments.

FIG. 39 depicts a review page for the overall claim submission according to some embodiments.

FIG. 40 shows a preconfigured package plan overview portion of the system according to some embodiments.

FIG. 41 illustrates an employer proposal user interface according to some embodiments.

FIG. 42 illustrates another aspect of the employer proposal user interface according to some embodiments.

FIG. 43 depicts a plan overview portion of the user interface according to some embodiments.

FIG. 44 shows an account summary portion of the employer portal system according to some embodiments.

FIGS. 45-59 depict non-limiting examples of a system generated Choice plan proposal according to some embodiments.

FIGS. 60-76 illustrate non-limiting examples of a system generated Select plan proposal according to some embodiments.

FIG. 77 shows a conditions list according to some embodiments.

FIGS. 78-107 illustrate non-limiting examples of an employee enrollment UI and process flow according to some embodiments.

FIGS. 108-112 illustrate non-limiting examples of enrollment shopping tips, limitations, and exclusions UI and process flow according to some embodiments.

FIGS. 113-114 show non-limiting examples of recommendation engine explanation and questions according to some embodiments.

FIG. 115 shows a non-limiting example of an employee account portal according to some embodiments.

FIGS. 116-134 depict non-limiting examples of an employee billing and payments portal UI and process flow according to some embodiments.

FIGS. 135-144 depict non-limiting examples of an employee profile portal UI and process flow according to some embodiments.

FIGS. 145-149 illustrate non-limiting examples of an employee plan info and policy documents portal UI and process flow according to some embodiments.

FIGS. 150-152 illustrate non-limiting examples of an employee reports portal UI and process flow according to some embodiments.

FIGS. 153-157 illustrate non-limiting examples of a member e-consent portal UI according to some embodiments.

FIGS. 158-183 illustrate non-limiting examples of a member claims portal UI and process flow according to some embodiments.

FIGS. 184-189 illustrate non-limiting examples of a member customer care messaging, help UI, and process flow according to some embodiments.

FIGS. 190-192 show non-limiting examples of the MyInfo portal UI according to some embodiments.

FIGS. 193-201 illustrate non-limiting examples of a beneficiary UI and process flow according to some embodiments.

FIGS. 202-204 illustrate non-limiting examples of an electronic consent UI and process flow according to some embodiments.

FIG. 205 illustrates a computer system 410 enabling or comprising the systems and methods in accordance with some embodiments of the system.

### DETAILED DESCRIPTION

In some embodiments, the system includes an innovative limited benefit fixed-indemnity product that provides benefits upon the diagnosis of specific conditions. In some embodiments, the system is one simplified product that provides wide-ranging protection across the spectrum of injuries and illnesses. In some embodiments, the system is designed to cover many more conditions than today's accident, critical illness, and hospital indemnity plans. In some embodiments, the system is not a bundled critical illness, accident, and hospital indemnity product. Rather, in some embodiments, the system is a single product that eliminates the need to string together these legacy product options. In some embodiments, the system provides a simpler employee-consumer experience and easier plan administration for employers. In some embodiments, in addition to insurance product innovation, the system creates a streamlined experience for members and employers.

In some embodiments, the system includes an International Classification of Diseases (“ICD”) conditions list. In some embodiments, the system is configured to sort the ICD conditions list into buckets according to treatment pathways and median patient out-of-pocket. In some embodiments, the system includes a proprietary coverage module configured to provide detailed pricing rates based on data from one or more guidelines (e.g., Milliman Health Cost Guidelines). In some embodiments, the system is configured to implement parametric claim adjudication based directly on ICD conditions specified in a contract.

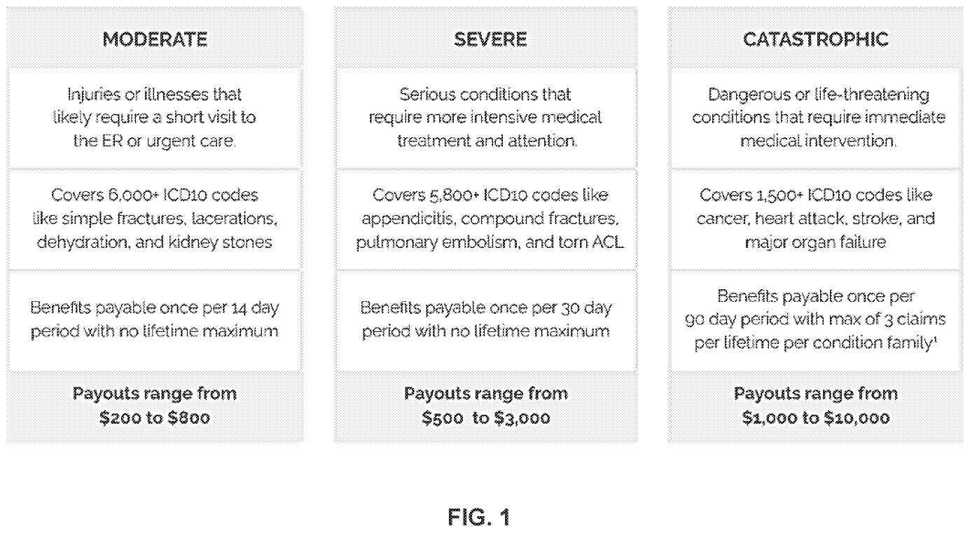

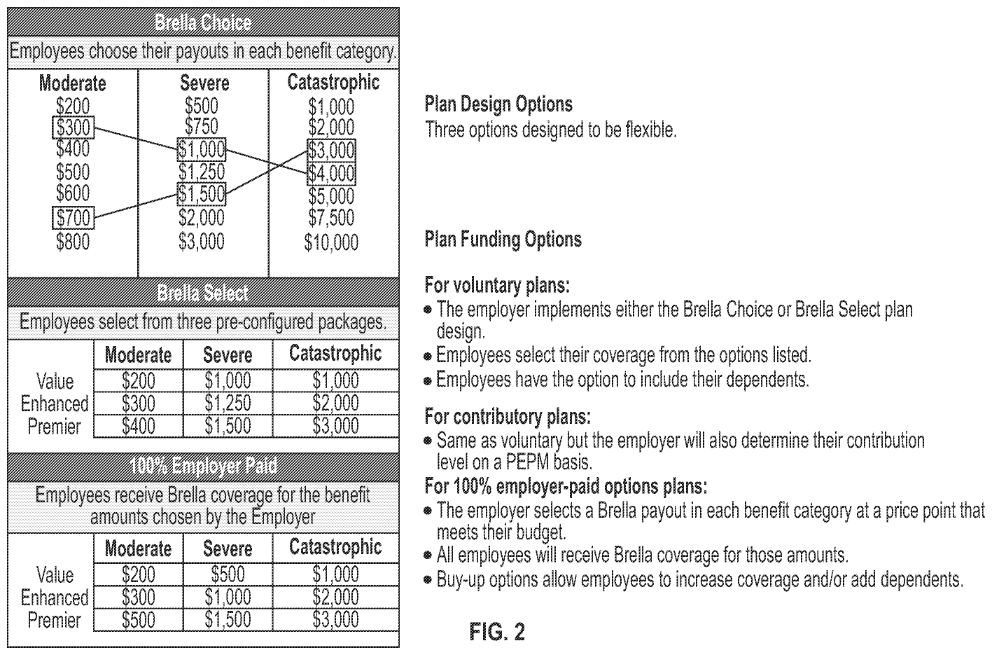

FIG. 1 illustrates an aspect of the system that includes a single product that includes three primary benefit categories: Moderate, Severe, and Catastrophic according to some embodiments. In some embodiments, the system is built for personalization and FIG. 2 offers at least two plan design options to provide employers and employees with flexibility, as well as depicts three plan design options in the form of Choice, Select and 100% employer paid according to some embodiments. In some embodiments, prior to enrollment, employers will select which of the plan design options they would like to offer to their employees. In some embodiments, with respect to the Select and 100% employer paid options, the system is configured to provide the ability to specify the number of plans, benefit amounts and other specifications.

In some embodiments, employees have the ability to add their eligible dependents to their plan at time of election. In some embodiments, coverage tiers include EE (Employee) only, EE+Child(ren), EE+Spouse, and Full Family. In some embodiments, the employer makes coverage available to eligible employees on either a 100% voluntary basis, or in conjunction with some level of employer funding.

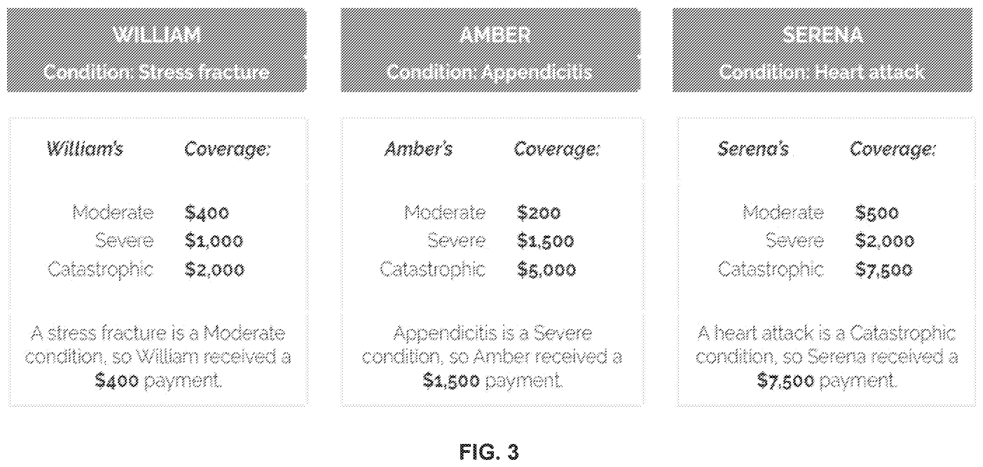

FIG. 3 shows the system applied to an example scenario that includes 3 different employees each with a different diagnosis and plan design configuration. In some embodiments, even though each condition is different, the system is configured to automatically determine the condition's category of coverage and the associated benefit amount. As with all examples presented herein, any number, value, or monetary designation are for illustration purposes only and the system is not limited to the values shown.

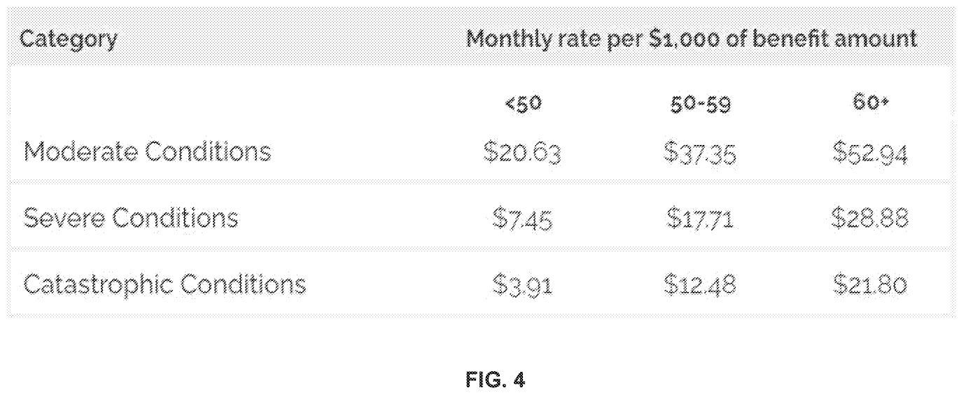

Plan configuration for the system includes rates according to some embodiments. In some embodiments, group underwriting applies. In some embodiments, employer rates are on a per $1,000 of covered benefit basis per coverage category. In some embodiments, age bands include <50, 50-59, and 60+(unisex rates), as non-limiting examples, as well as any other age bands determined to be appropriate for a particular type of coverage. In some embodiments, an employee's age and gender is used for rate and/or premium calculations. In some embodiments, composite rates can apply in exception cases. FIG. 4 shows some sample age-banded rates according to some embodiments. In some embodiments, if the system's Select plan design is chosen by the employer, the system is configured to generate one or more of age-banded monthly calculated premiums for value, enhanced, and premium per coverage tier. In some embodiments, plan offerings and rates are accessed by third parties via an API.

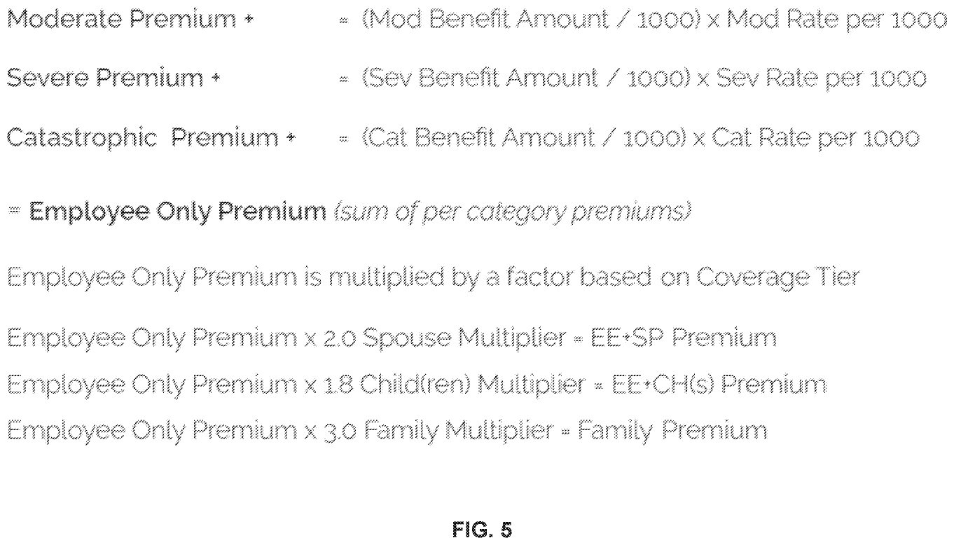

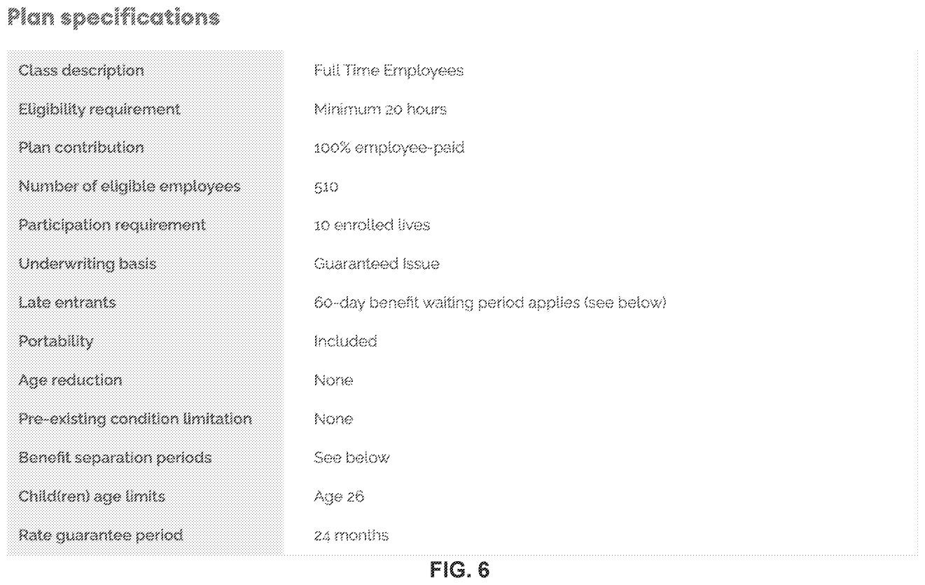

FIG. 5 illustrates example premium calculations according to some embodiments. In some embodiments, monthly premiums are calculated based on the employee's attained age. In some embodiments, benefit amounts are multiplied by the associated rate per $1000 per category. FIG. 6 shows example plan rules and specifications according to some embodiments.

In some embodiments, the system is built on a flexible technology stack configured to support multiple integration formats. In some embodiments, the system uses a proposed standard format for enrollment and eligibility maintenance. In some embodiments, the system streamlines delivery of new and/or renewal sold case data in a variety of formats as well. In some embodiments, the system is configured to update rates or plan configurations for renewal case data each year.

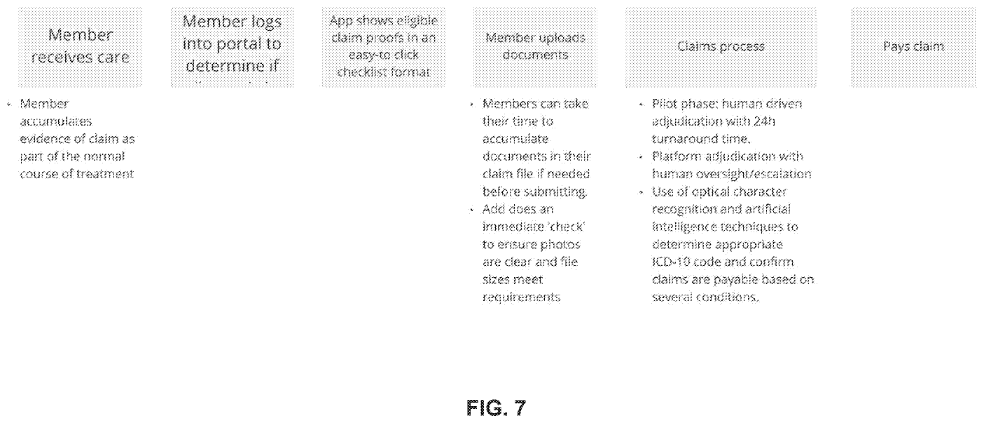

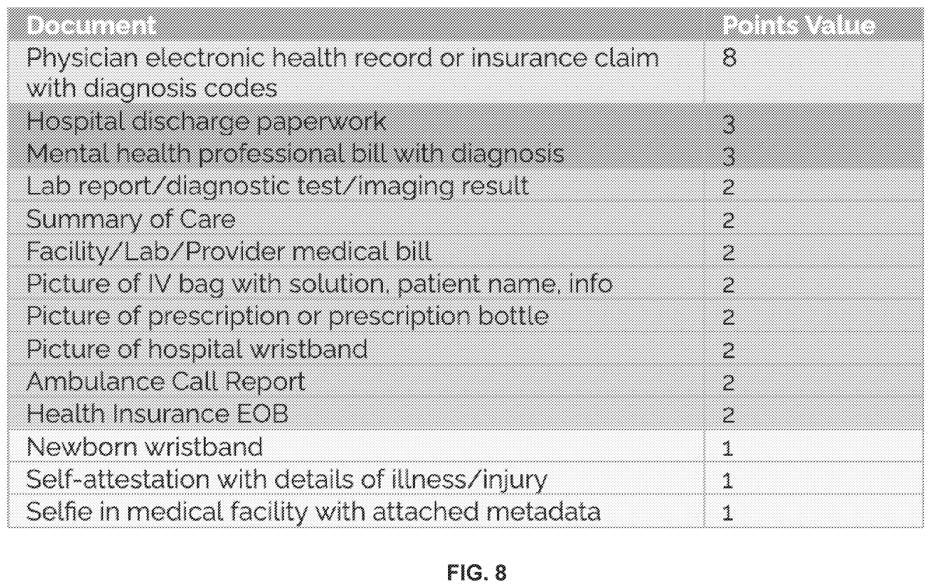

FIG. 7 shows a portion of the claims module process flow according to some embodiments. In some embodiments, filing claims relies on documents the claimant has readily at hand. In some embodiments, the claims module includes a claims submission process that is guided. In some embodiments, the system enables claimants to swiftly know whether their claim is eligible or not. In some embodiments, the claims module includes a points value for each eligible document to enable the automatic coverage determination. FIG. 8 depicts a non-limiting example of points assigned to different documents according to some embodiments. In some embodiments, the system is configured to receive medical claims data from a health insurer or third party administrator (e.g., for self-funded employers). In some embodiments, the system is configured to proactively pay claims without the claimant needing to file a claim.

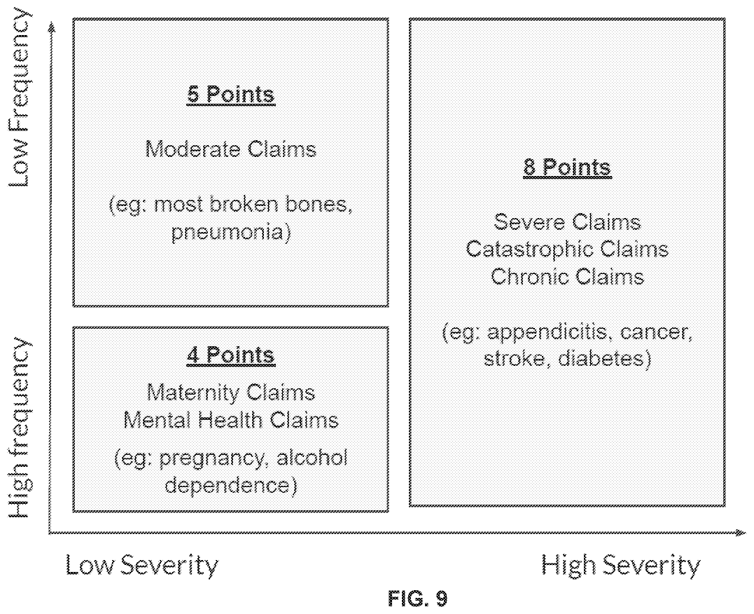

FIG. 9 depicts a claim point value for each condition group according to some embodiments. In some embodiments, each condition group is assigned a required claim point value by the claims module. In some embodiments, the claims module is accessed through a secure network comprising employing cryptology services. In some embodiments, the member portal module comprises the claims module. In some embodiments, the secure processing platform comprises the claims module. In some embodiments, the claims module is configured to enable consumers to submit any combination of eligible claim documents to accumulate enough points to submit a claim. In some embodiments, the claims module is configured to automatically identify text in a document or in a photograph and convert it to digital form. In some embodiments, the system is configured to automatically identify medical equipment (e.g., prescription bottles, casts, wristbands, IV bags, anatomical structures, and/or any physical structure related to a medical diagnosis or procedure) in images and/or videos submitted by a patient. In a non-limiting example of this functionality, in some embodiments, a patient may submit an image of a prescription bottle. In some embodiments, the claims module then analyzes the image and determines that the image is of a prescription bottle by comparing the image to training and/or historical images in a database. Simultaneously or in a previous or subsequent step, in some embodiments the claims module applies one or more artificial intelligence (“AI”) text recognition techniques to the image to identify text printed on the prescription bottle label. The image's structure and/or text obtained from the image is then used by the claims module's AI algorithm to determine an ICD 10 code and/or the appropriate category according to some embodiments.

In some embodiments, the claims module is configured to automatically extract medical information (e.g., electronic medical records) from one or more third party data sources. In some embodiments, the extracted medical information is analyzed by the claims module to aid in determining the validity of a claim. In some embodiments, the claims module is configured to use the extracted medical information to augment evidence supplied by the member. In some embodiments, the system is configured to automatically identify a covered condition and/or automatically pay a claim based on the extracted medical information, saving both man-hours and computer resources. In some embodiments, the system is configured to automatically validate whether a claim is payable based on coverage dates, condition categories included in the coverage, the separation periods of prior claims, among other automatic checks.

In some embodiments, AI-assisted algorithms are used to identify text, forms, and/or medical equipment. In some embodiments, the claims module is configured to use AI to confirm the validity of submitted evidence for claim approval. As used herein, AI is a broad term that encompasses all conventional systems and methods that allow a machine to learn from data. In some embodiments, the claims module is configured to extract and/or determine the proper ICD 10 code from the eligible claim documents to determine categorization as further described below. In some embodiments, AI is used to extract and/or determine the proper ICD-10 code from analysis of eligible claim documents, including images of associated medical equipment (e.g., a cast). In some embodiments, the claims module includes an all-digital workflow that enables a verbose data collection process for all user touch points to train the AI. In some embodiments, the data is transactional and clickstream. In some embodiments, the touch points include one or more of: (1) performance marketing; (2) group lead qualification; (3) quote/proposal; (4) underwriting; (5) enrollment; (6) mobile claims app and member portal; (7) fraudster behavioral data. In some embodiments, the AI models are applied to one or more of (1) lead qualification including risk and/or loss ratio prediction; (2) pricing including group risk based pricing; (3) personalization including precision plan selection and payout levels; (4) customer and member experience including Net Promoter Score (“NPS”); (5) claims including image submission quality improvement, ICD-10 extraction, fraudulent document detection, and/or auto-adjudication for speed; and/or (6) reserve including algorithmic precision on reserve amounts.

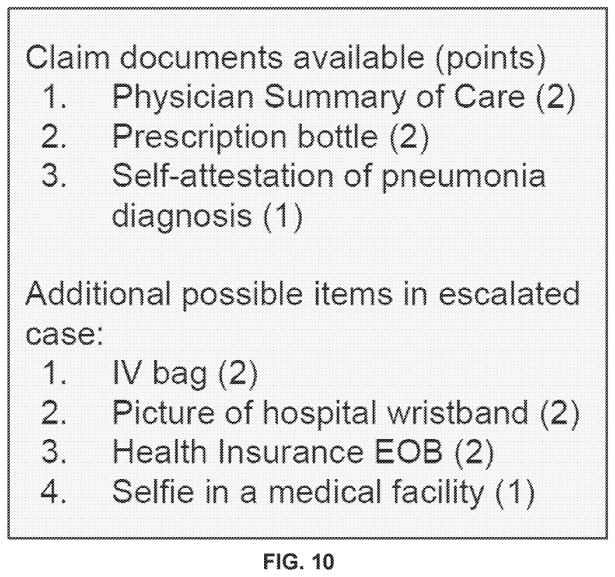

In some embodiments, an example customer experience for pneumonia includes: (1) A Primary Care Physician (“PCP”) visit for persistent cough/trouble breathing/chest congestion; (2) receiving antibiotics for 14 days; and/or (3) a follow-up visit in 2 weeks, but call if not better in 2 days. In some embodiments, if the pneumonia is not resolved, the patient experience may include: (1) adjusted antibiotic regimen; (2) outpatient daily antibiotic treatment; and/or (3) inpatient IV treatment with antibiotics and fluids. FIG. 10 depicts example eligible claim documents for a moderate (5 pts) claim submission for pneumonia according to some embodiments.

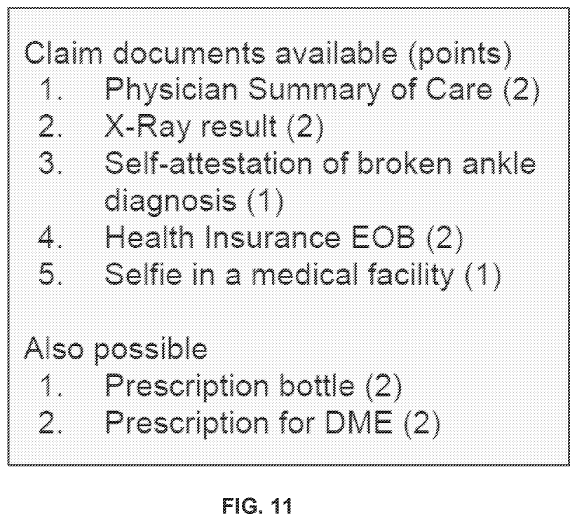

In some embodiments, an example customer experience for a broken ankle includes: (1) a visit to urgent care; (2) X-ray to confirm broken ankle diagnosis; (3) receiving a hard cast or walking cast; and/or (4) a follow-up appointment in 6 weeks. In some embodiments, the customer experience includes a prescription for pain and/or physical therapy. FIG. 11 depicts example eligible claim documents for a moderate (5 pts) broken ankle claim submission according to some embodiments.

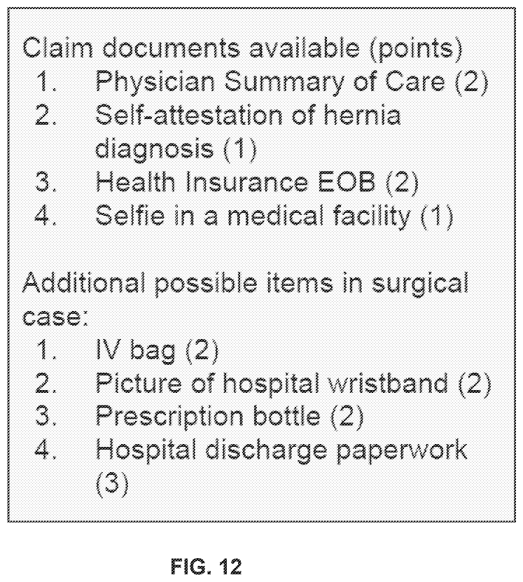

In some embodiments, an example customer experience for an inguinal hernia includes one or more of: (1) a visit to a PCP; (2) receiving a conservative “watch and wait” diagnosis; (3) surgeon visit; (4) same-day surgery; (5) prescription for pain and/or antibiotic; and/or (6) a follow-up appointment for 2 weeks and 6 weeks. FIG. 12 depicts example eligible claim documents for a severe (8 pts) broken ankle claim submission according to some embodiments.

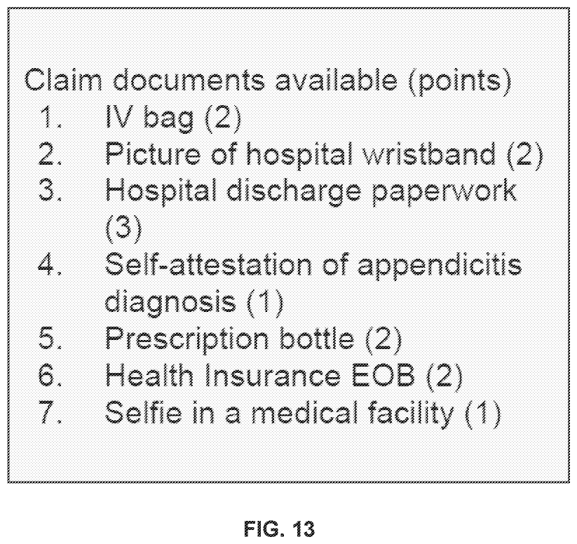

In some embodiments, an example customer experience for appendicitis includes one or more of: (1) a visit to the emergency room (“ER”) with severe abdominal pain; (2) imaging to confirm appendicitis diagnosis; (3) surgery to remove the appendix; (4) prescription for pain and antibiotic; (5) home health care; and/or (6) follow-up with physician in 2 weeks. FIG. 13 depicts example eligible claim documents for a severe (8 pts) appendicitis claim submission according to some embodiments.

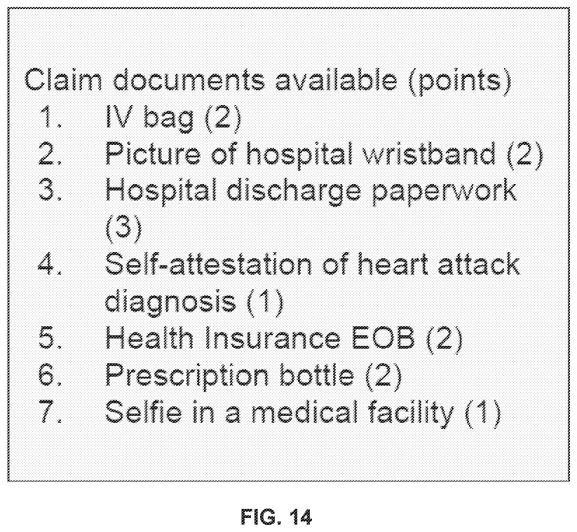

In some embodiments, an example customer experience for a heart attack includes one or more of: (1) emergency room visit; (2) resuscitation; (3) electrocardiogram (“EKG”) to diagnose heart attack; (4) blood test to confirm diagnosis; (5) monitoring for abnormal heart rhythm; (6) cardiac catheterization procedure; (7) inpatient hospital stay; (8) hospital discharge; (9) physician follow-up; and/or (10) cardiac rehab. FIG. 14 depicts example eligible claim documents for a severe (8 pts) heart attack claim submission according to some embodiments.

In some embodiments, the system includes one insurance product that pays a lump-sum for 13,000+ covered medical conditions. In some embodiments, the system provides automatic approval and/or dispersion of funds over a secure (encrypted) data transfer network for approximately 76% of conditions that would require emergency medical attention, which is 3 times what traditional critical illness and accident plans cover combined, noting that no traditional plans offer the automatic approval and/or dispersion of funds provided by the system.

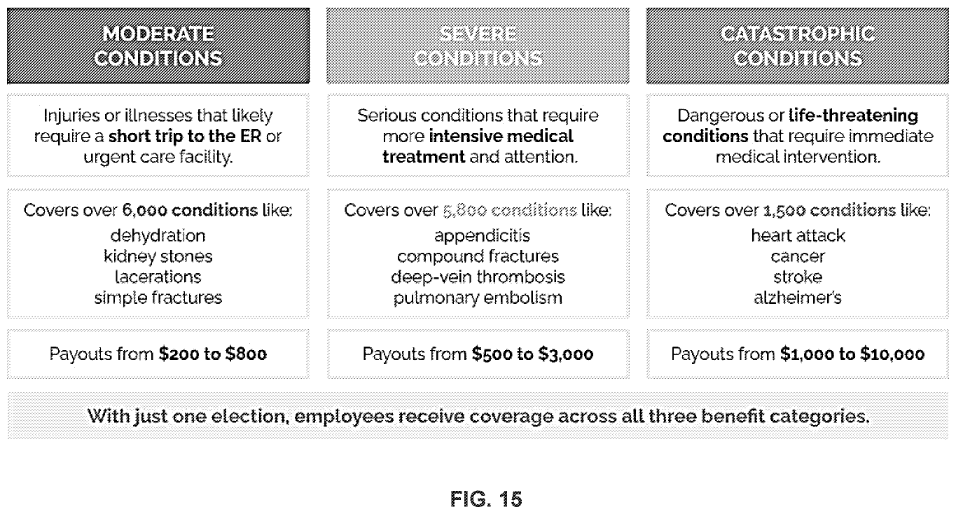

To achieve the automatic approval and/or dispersion of funds, the claims module uses a combination of ICD-10 (diagnosis) codes, and distinct benefit categories (e.g., 3, although any number of categories are contemplated) to determine eligibility to be paid a benefit. Unlike the prior art, some embodiments of the system do not require human intervention, approval, and/or review which provides the following benefits over the prior art: (1) lower cost for the insurance company in terms of person-hours; (2) substantial reduction in network communication bandwidth usage from individuals on both the patient and insurance side transferring, accessing, and reviewing claim documents both verbally based on analog media as well as digitally; (3) reduction in computer storage by reducing the number of forms and/or evidence needed for claims; and (4) faster payout to patients which limits exposure to financial hardships. According to some embodiments, in the secure processing platform and/or the claims module, the ICD-10 code and/or condition is determined automatically using one or more eligible documents as described above. FIG. 15 provides a summary for some embodiments of the system with 3 distinct benefit categories (Moderate, Severe, and Catastrophic) and covered conditions that correlate to ICD-10 codes.

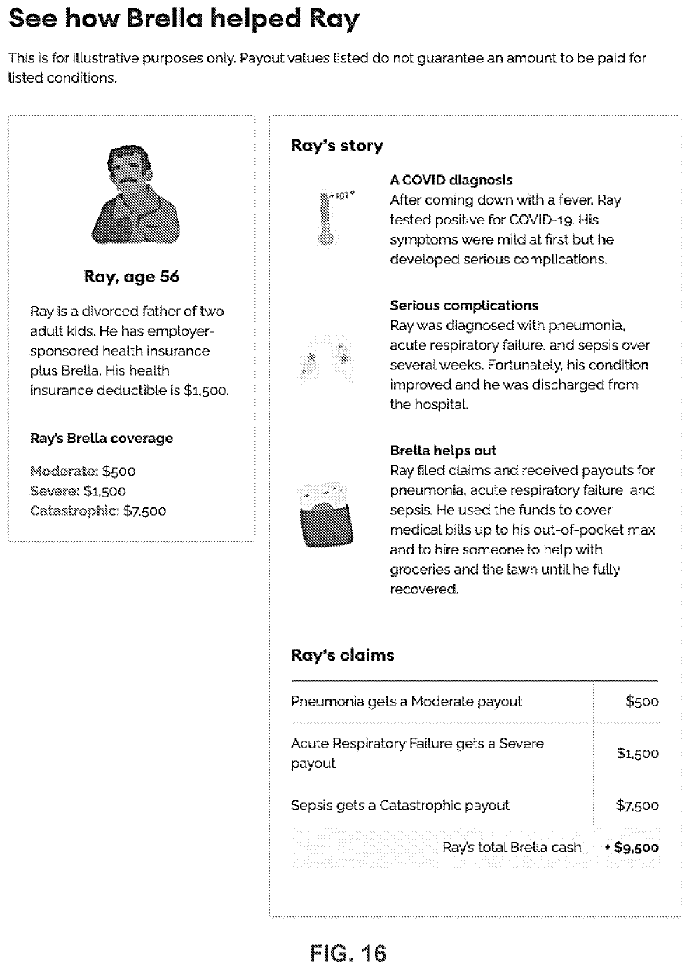

In some embodiments, the claims module is configured to determine if more than one category is covered by analysis of one or more eligible documents submitted by a member. Since the system is based on categorization of diagnosis, the claims module is configured to identify individual diagnosis in one or more eligible documents and assign each diagnosis to the appropriate category. Advantageously, in some embodiments the claims module and/or digital marketplace module is configured to distribute corresponding benefits over one or more secure connections for each diagnosis according to each category. In some embodiments, the system is configured to send the distribution over a secure connection in separate payments. In some embodiments, the claims module is configured to consolidate all benefits from each category into a single payment, which is distributed by the digital marketplace module according to some embodiments. In some embodiments, the claims module is configured to determine whether to group payments or pay individually based on the time frame between when documents are received. FIG. 16 represents an example of multiple category determination and payout according to some embodiments. In this example, although the initial diagnosis was not a covered condition, the system is configured to automatically identify secondary diagnoses of covered conditions from submitted eligible documents resulting in multiple categorical claim payouts.

In some embodiments, the system enables claim submission by mobile app or web accessed member portal GUI. In some embodiments, the system enables paperless claim submission in minutes. In some embodiments, the system enables the use of photos to submit claim evidence. In some embodiments, a bill from a physician is not needed to file a claim. In some embodiments, the system enables payment of benefits directly to a bank account and/or third-party payment service (e.g., PayPal®, Venmo®).

In some embodiments, the system is configured to use a member's GPS location to notify and/or remind the patient of potential coverage. For example, in some embodiments, if a member's location is at or near (e.g., within 200 ft of) a hospital, the system is configured to send a notification (e.g., text, email, or any other notification) over an encrypted network with instructions for how to submit a claim. In some embodiments, the system is configured to use a member's location data in conjunction with public information about the location to send specific instructions and/or coverage information to the patient. For example, if the member is at a location listed as an orthopedic specialist office on Google Maps®, the system is configured to send a link to covered bone injuries and/or documentation to the member according to some embodiments. This is advantageous as there are over 13,000 conditions covered by the system, most of which are not covered by traditional plans and narrowing down potential conditions saves the member time and increases the probability of a payout. In addition, in some embodiments, the system is configured to provide the member with example eligible claim documents specific to the type of medical setting the member visited. This further improves the system analysis as the system is configured to suggest the best documentation and format for upload to ensure that all eligible conditions are identified.

In some embodiments, the system includes a partner portal module for agency set-up, partner onboarding and management, quote request, and employer client management. In some embodiments, the system includes an underwriting module and custom rating engine (module). In some embodiments, the system includes an interactive proposal tool for plan option visualization and employer configuration. In some embodiments, the system includes straight-thru employer account creation from proposal to application e-signature. In some embodiments, the system includes auto-generation of policy and certificate documents with conditional variables.

In some embodiments, the system includes an automated underwriting module. In some embodiments, the automated underwriting module is configured to accept one or more underwriting inputs from a UI (e.g., inputs from a partner and/or employer). In some embodiments, the automated underwriting module is configured to obtain census data for an individual and/or a group of individuals from one or more proprietary and/or one or more third-party databases. In some embodiments, census data includes one or more of age, gender, SIC code, credit score, address, biometric data, criminal records, behavioral data, and/or any conventional data used in the underwriting process. In some embodiments, the automated underwriting module is configured to estimate risk for one or more of each individual and/or a group comprising each individual for the risk assessment. In some embodiments, the automated underwriting module is configured to generate a score based on the estimated risk for each individual and/or the group. In some embodiments, if a risk is determined to be high, the automated underwriting module is configured to notify the user that a quote cannot be provided. If a risk is assessed as medium, the automated underwriting module is configured to request additional information to further assess risk and/or is configured to supply a quote proportional to the risk assessment according to some embodiments. In some embodiments, if the risk is determined to be low, the automated underwriting module is configured to automatically provide a quote proportional to the risk assessment. In some embodiments, automatically providing a quote without human intervention saves both man-hours and computer resources, as well as increases profit margins as risk is more accurately reflected in the quote. In some embodiments, once the automated underwriting module has determined risk and provided a quote, the system is configured to automatically set up a case and/or automatically begin an open enrollment process. In some embodiments, the system is configured to enable partners and/or their underwriters to access and/or administer the automated underwriting module. In some embodiments, the access is over a secure network comprising an encrypted data transfer program.

In some embodiments, the system includes an all-in-one education, recommendation and enrollment/shopping platform. In some embodiments, the system includes an employee shopping platform, including multilingual support. In some embodiments, the system includes an employer portal for member management and ongoing administration. In some embodiments, the system includes a member portal generated by a member portal module for ongoing account management and claim submission through a GUI. In some embodiments, the system includes an E-payment for premium collection and claim payments (PayPal®, Stripe®, Venmo®, or similar E-payment systems, and/or ACH straight to bank account transfer). In some embodiments, the system includes flexible billing and transaction management for premiums. In some embodiments, the system includes member mobile applications for Android® and IOS® to access one or more system modules including the member portal module described herein.

In some embodiments, the system includes a digital marketplace module. In some embodiments, the digital marketplace module is configured to direct claim payments from the claims module to a digital wallet. In some embodiments, the digital marketplace module is configured to disperse funds for medical related expenses for products and/or services related to a member's medical condition. In some embodiments the digital marketplace module is configured to automatically determine qualified purchase items (e.g., prescriptions, medical equipment, and/or any conventional medical items) by analysis of the documents supplied by the member and/or documents obtained from a third-party database (e.g., electronic medical records). In some embodiments, the digital marketplace module is configured to search and compare prices of the qualified purchase items and automatically provide the lowest cost option to the member. In some embodiments, the digital marketplace module is configured to automatically purchase the qualified purchase items and/or provide delivery information to third-party vendors.

In some embodiments, the system includes a financial module. Like every module described herein, the financial module is configured to interface with one or more other module and/or associated data through the secure processing platform and/or some other central platform. In some embodiments the financial module is configured to automatically negotiate bills with providers (throughout this disclosure, the term “provider” includes any entity that provides medical equipment and/or services). In some embodiments, the financial module is configured to analyze proprietary and/or third-party databases over one or more encrypted networks to determine the lowest cost for a procedure and/or service. In some embodiments, the financial module is configured to automatically generate a negotiation notification and/or deliver the negotiation notification to one or more providers. In some embodiments, the negotiation notification includes an “accept” and/or “decline” link and/or button. In some embodiments, the financial module is configured to automatically pay a provider over a secure network who accepts the automatically generated negotiation notification. In some embodiments, the financial module is configured to provide a member with a quote for a low interest loan to cover medical expenses. In some embodiments, the financial module is configured to automatically populate documents relating to loan approval using member information.

In some embodiments, the system (e.g., secure processing platform) includes a recommendation engine. In some embodiments, the recommendation engine includes one or more user interfaces (“UIs”) configured to accept one or more inputs. In some embodiments, the recommendation engine (recommendation experience GUI) is configured to accept one or more of the following inputs from a customer: (1) age; (2) spouse; (3) has child dependents; (4) qualified high-deductible health plan (“QHDHP”); and (5) discretionary income. As used herein, a customer includes an employee of a company or an individual purchasing insurance directly (i.e., not through an employer): the system is able to be used by any individual and/or groups of individuals according to some embodiments. In some embodiments, the recommendation engine is configured to accept one or more of the following inputs from an admin: (1) price tolerance limit and (2) package adjustment method.

In some embodiments, the recommendation engine is configured to automatically obtain personal information for an individual and/or a group of individuals from one or more proprietary databases and/or one or more third-party databases. In some embodiments, personal information, secured over one or more encrypted networks, includes one or more of age, gender, SIC code, credit score, address, biometric data, criminal records, behavioral data, and/or any conventional data used to determine coverage pricing. In some embodiments, the recommendation engine is configured to use at least a portion of the obtained personal information together with one or more supplied inputs to determine coverage pricing.

In some embodiments, the recommendation engine is configured to compare the price of a recommended package to the price tolerance limit. In some embodiments, if the recommended package price is greater than the price tolerance limit, the system is configured to execute one of: (1) lower the benefits for a given package and (2) reduce the benefit within a given package that was statistically chosen the least. In some embodiments, the recommendation engine is configured to generate one or more of the following outputs: (1) coverage tier default; (2) benefit levels recommendation; (3) monthly price; and (4) recommendation “why” text.

In some embodiments, the recommendation engine is configured to influence factors for each benefit type based on one or more user input variables. In some embodiments, the recommendation engine is configured to correlate the input variables to multiplier factors from one or more Individual Factor Tables (“IFT”). Examples according to some embodiments of the IFT applying multiples to the one or more user input variables for a customer in the 60-64 age range include: (1) Catastrophic=2.0: where the IFT increases the factor with age; (2) Severe=1.0: where the IFT increases the factor with age, but not as much as for the Catastrophic category; and (3) Moderate=0: where the recommendation does not change with age.

In some embodiments, the recommendation engine is configured to sum the individual influence factors for each benefit level (e.g., for Moderate, the system sums up the influence of Age+Has Child Dependents+“Qualified High Deductible Health Plan (“QHDHP”)). In some embodiments, each summation correlates to a Benefits Conversion Table, where each sum represents the number of steps up or down to move the starting package amounts. For example, if the Catastrophic sum for a given user is 2.0 and the starting Catastrophic benefit amount is $4,000, the system is configured to recommend a benefit amount two steps up, or $8,000 according to some embodiments.

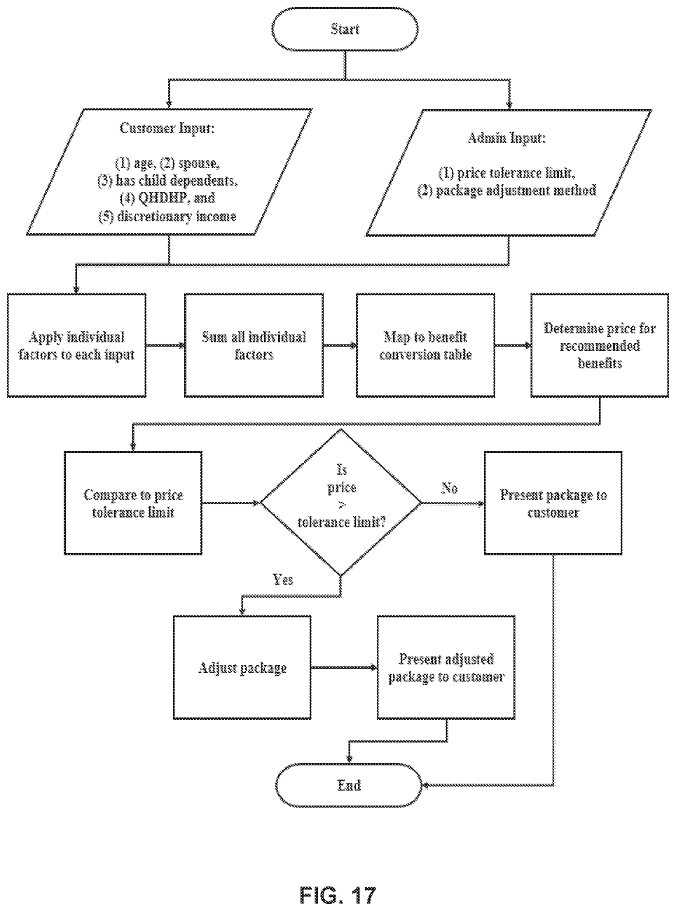

In some embodiments, once the recommendation engine formulates a recommended benefit amount for each benefit type, the system (secure processing platform) is configured to determine a proposed price for a package. In some embodiments, the system is configured to compare the recommended package price to the price tolerance limit as described above and recommend a new package to the customer. FIG. 17 shows a flowchart for an example recommendation engine execution according to some embodiments. All flowcharts presented herein represent computer implemented steps and/or are visual representations of algorithms implemented by the system.

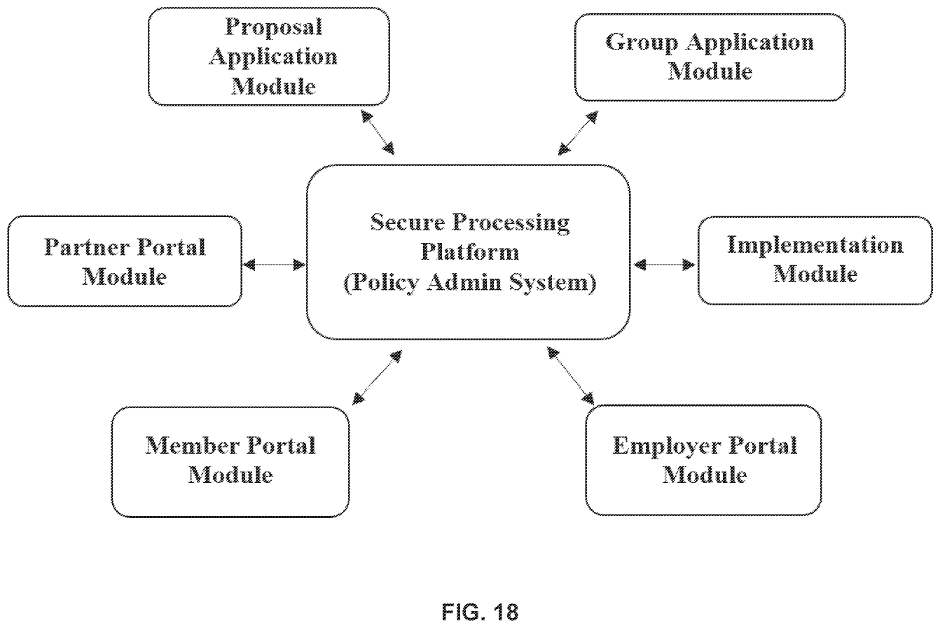

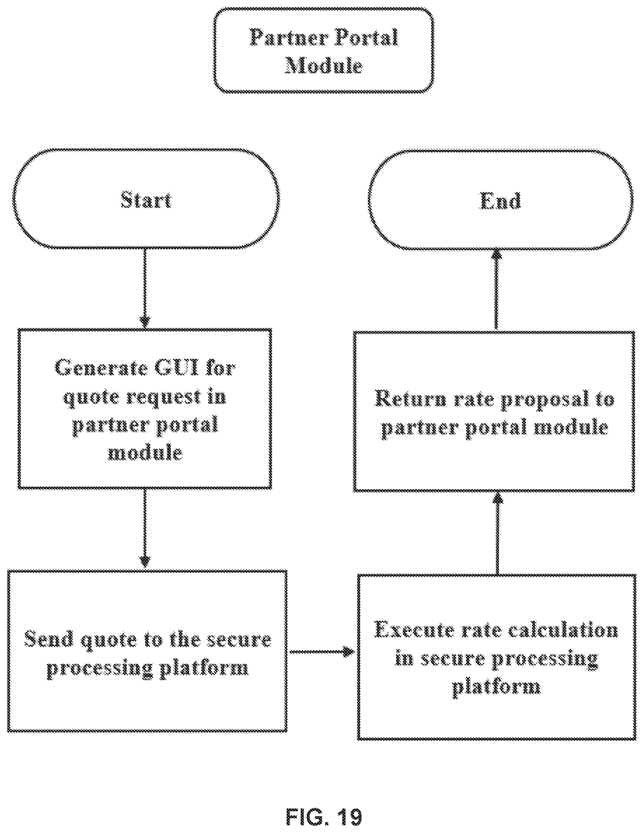

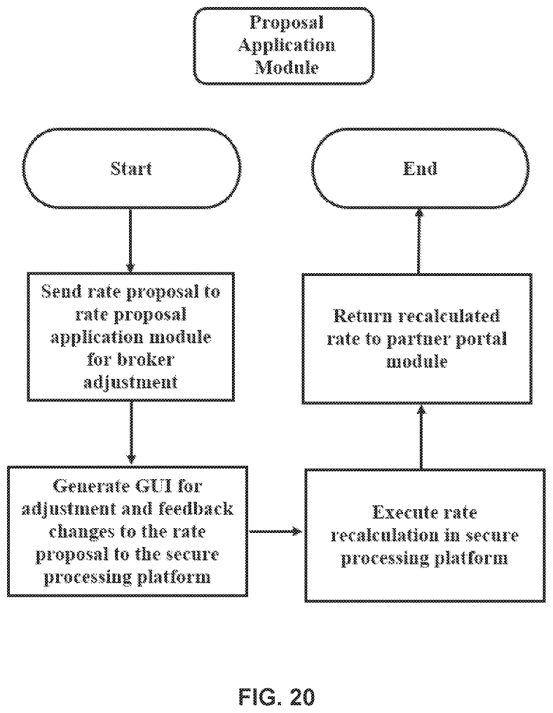

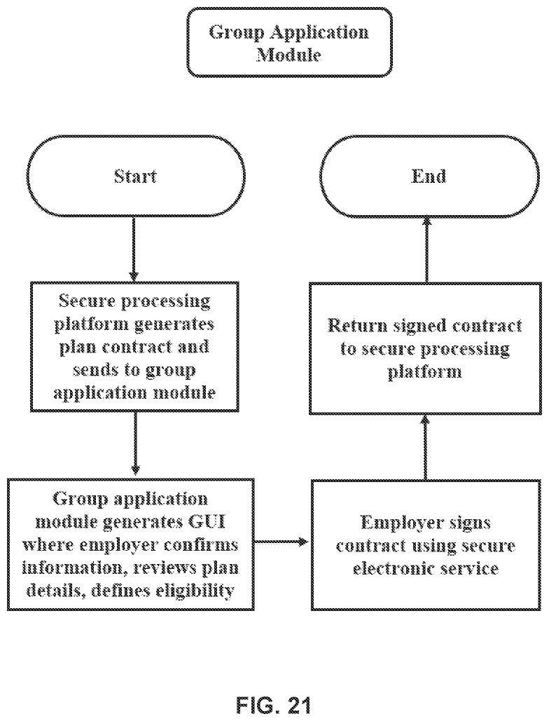

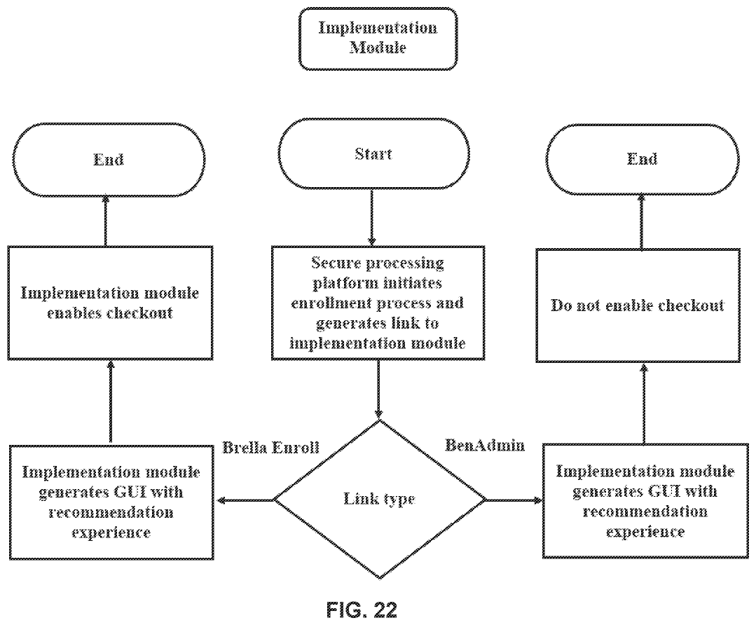

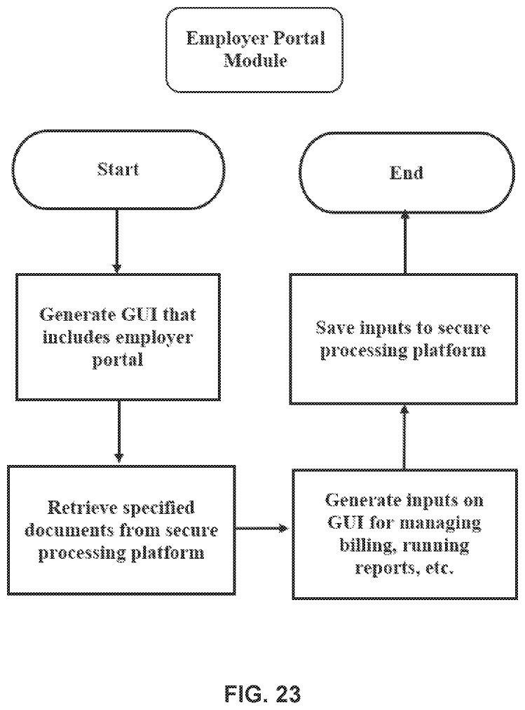

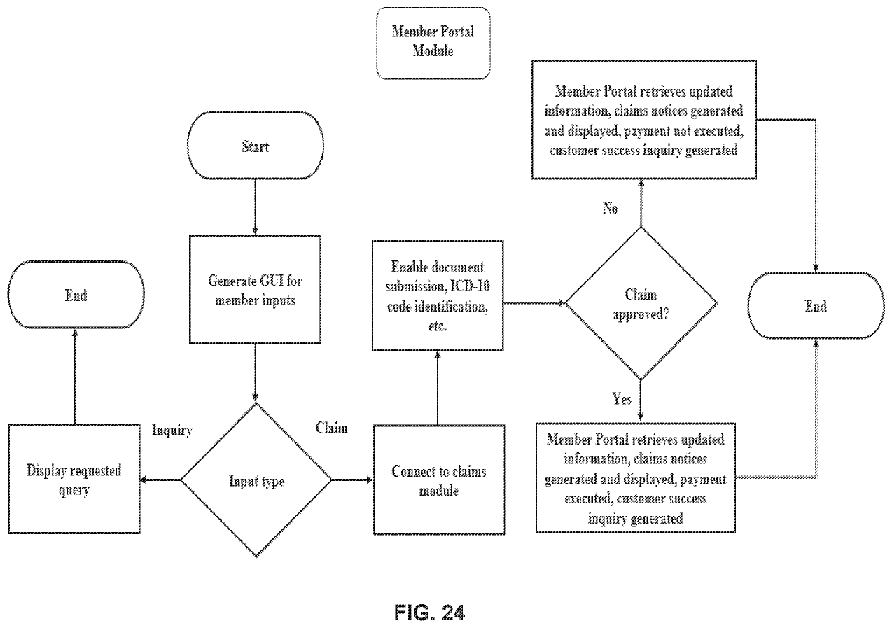

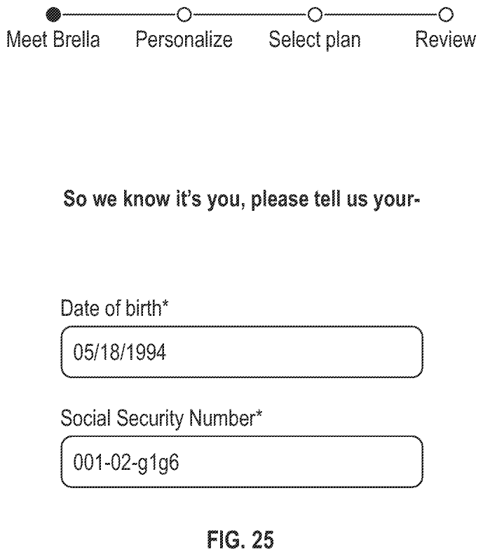

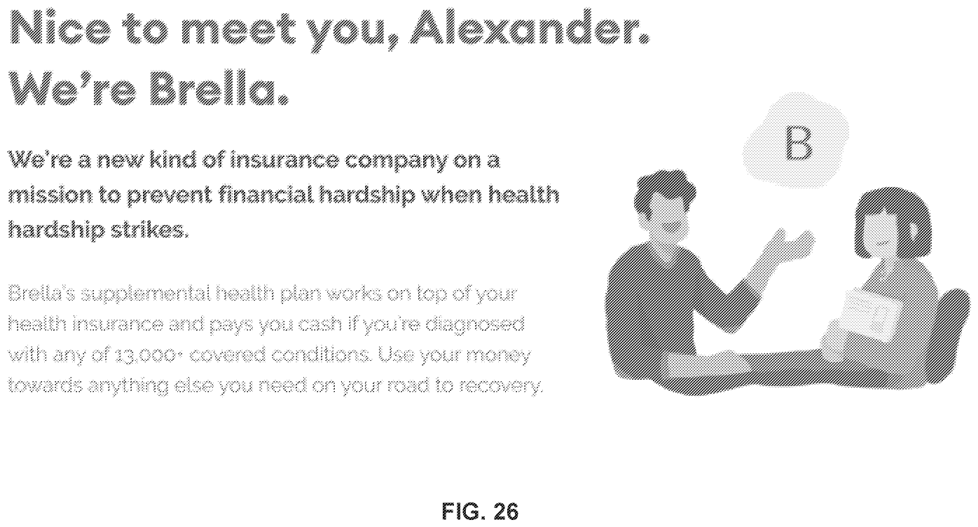

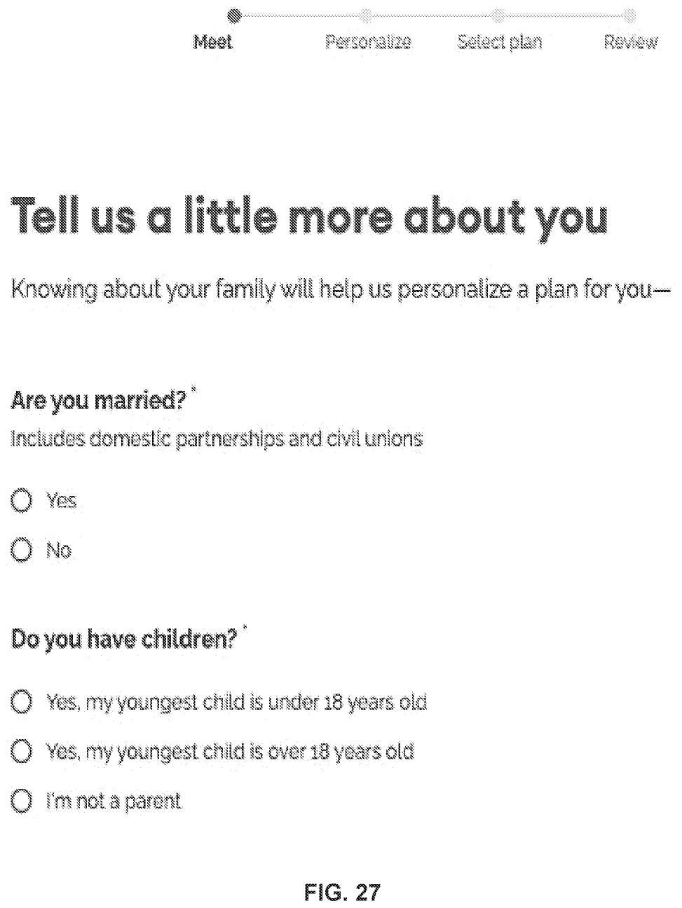

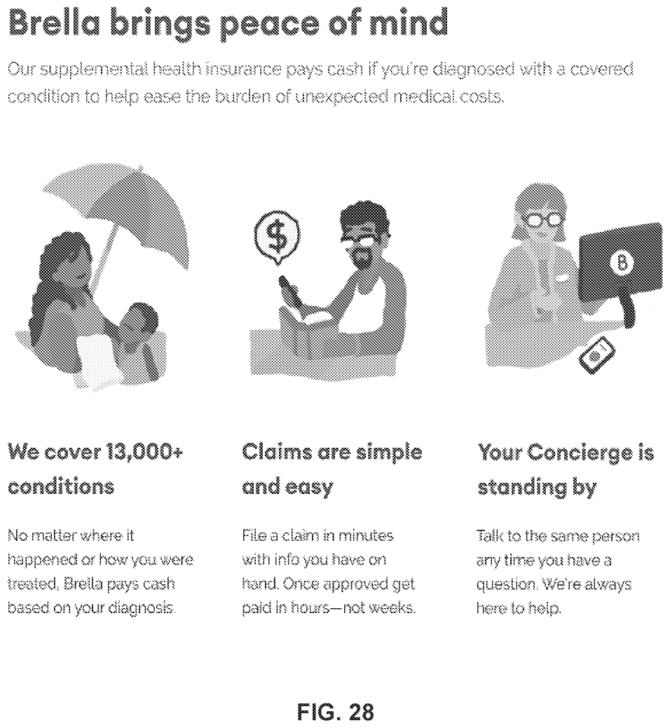

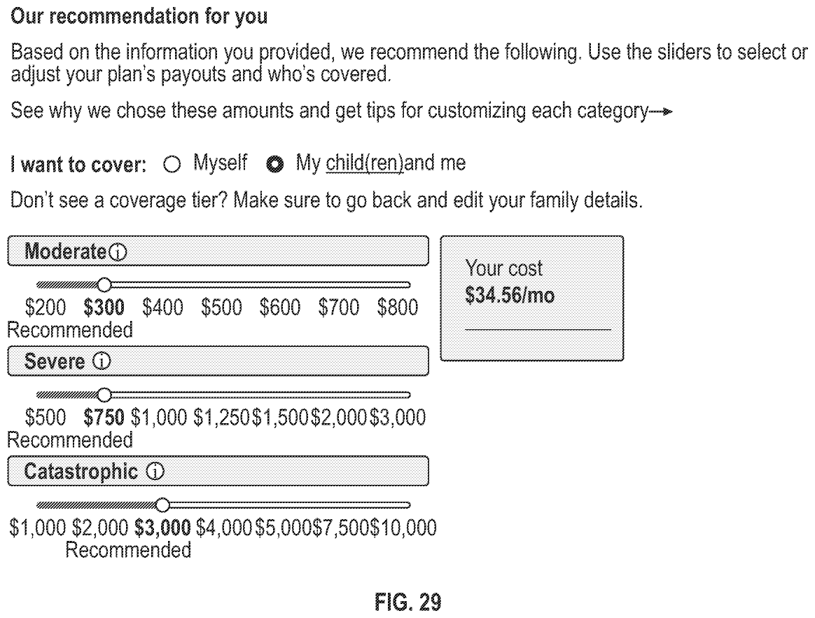

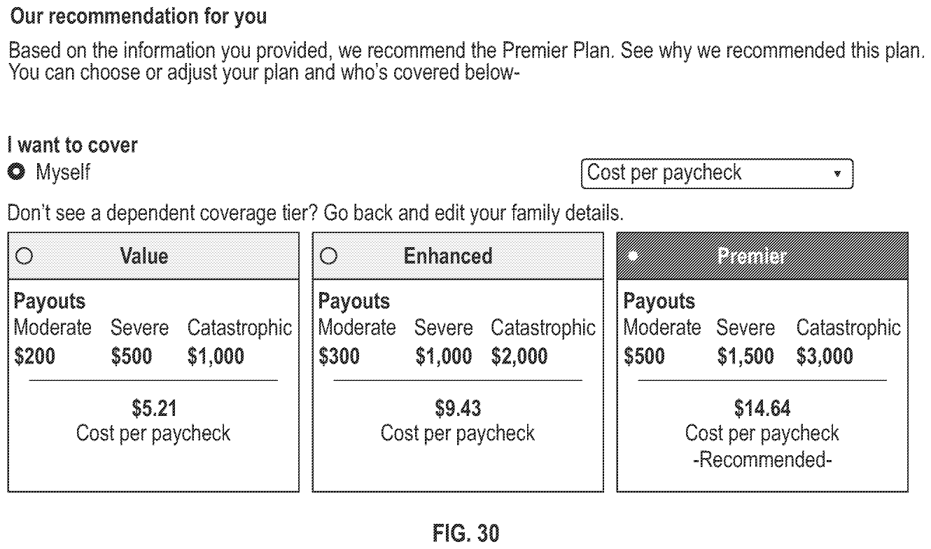

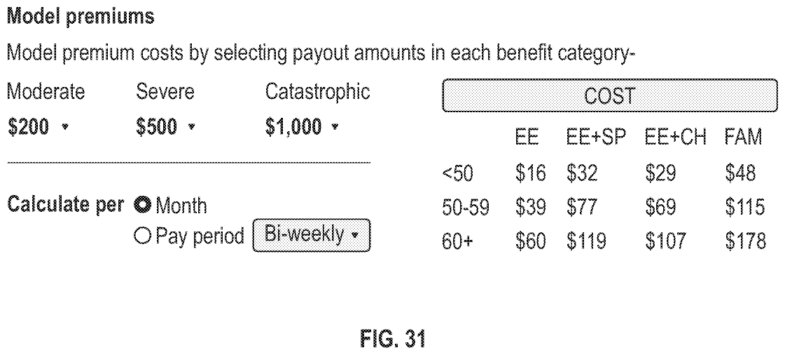

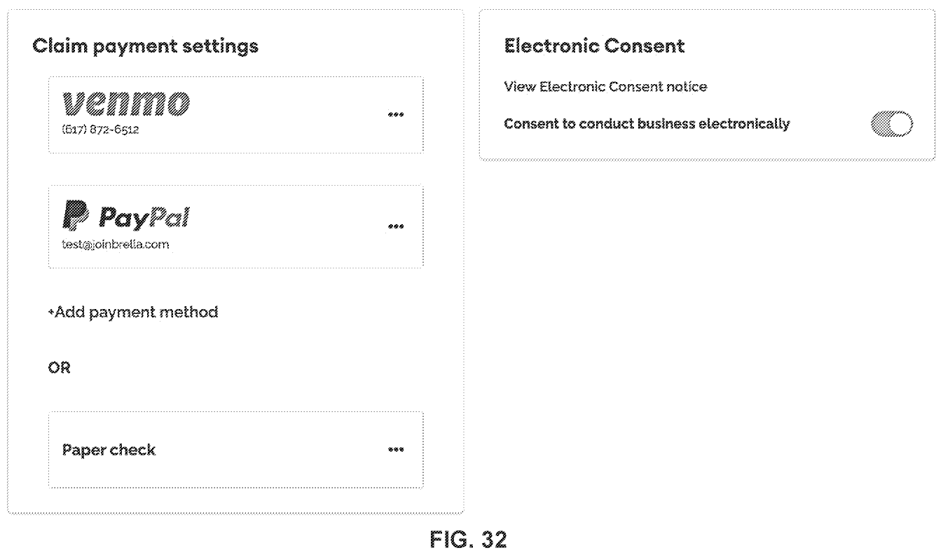

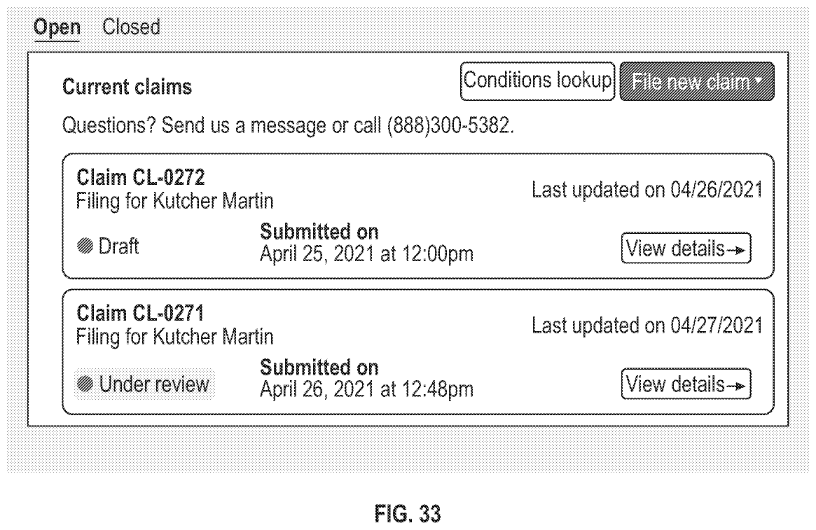

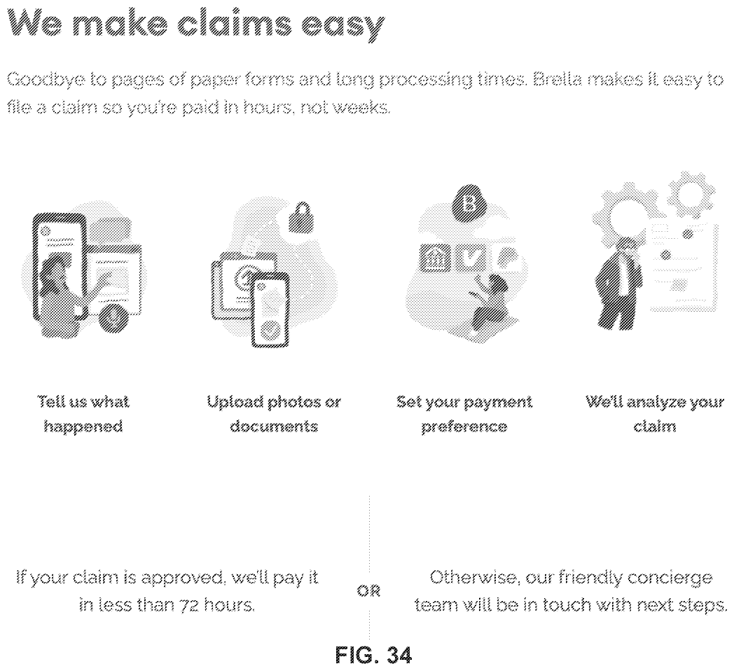

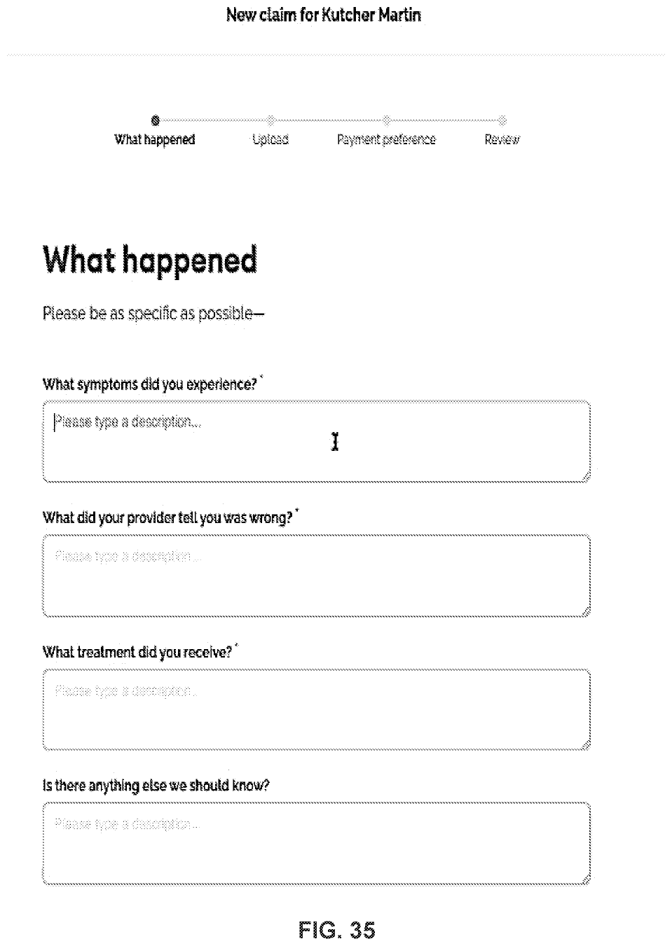

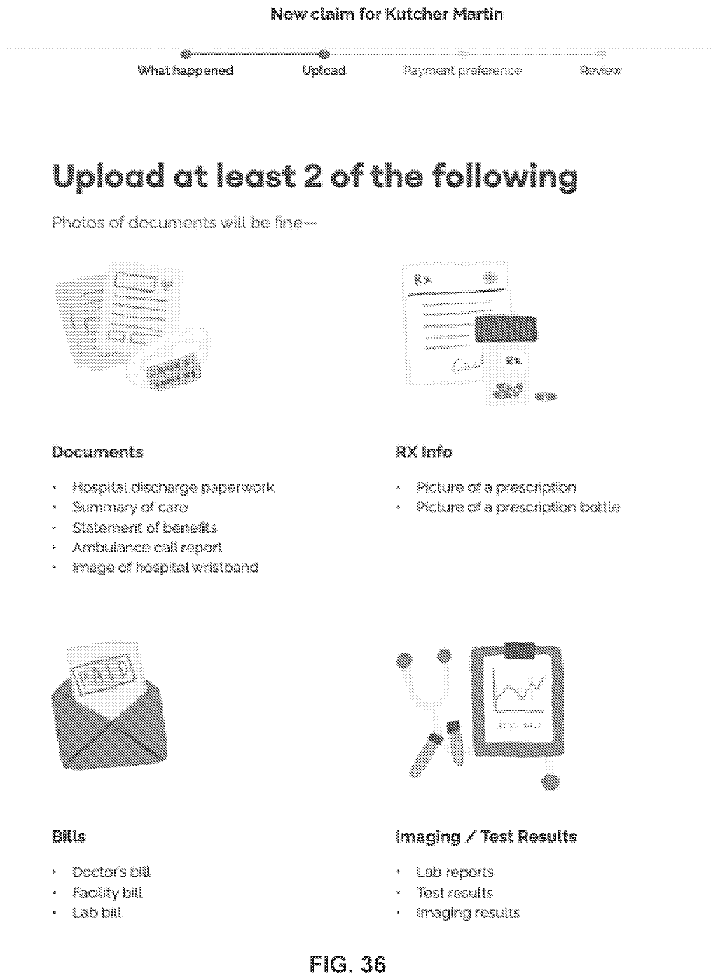

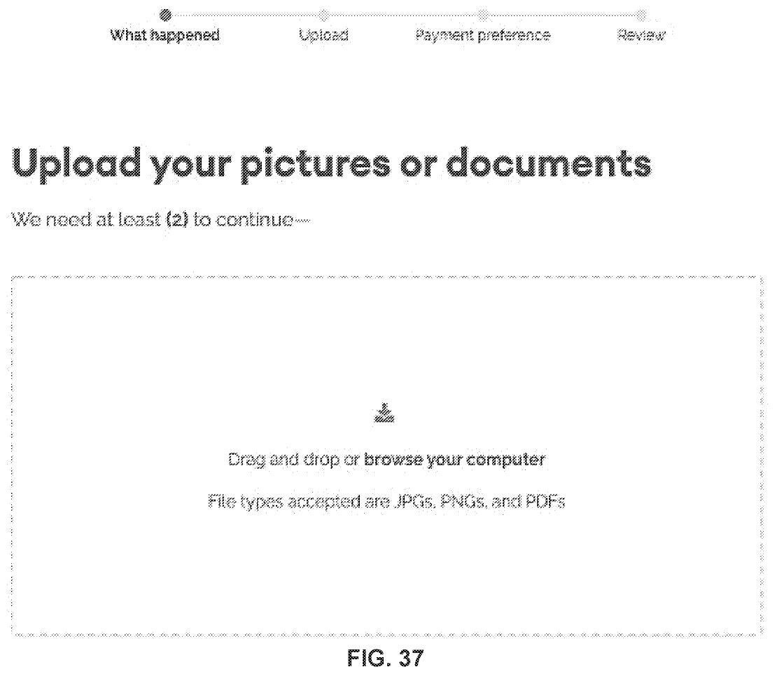

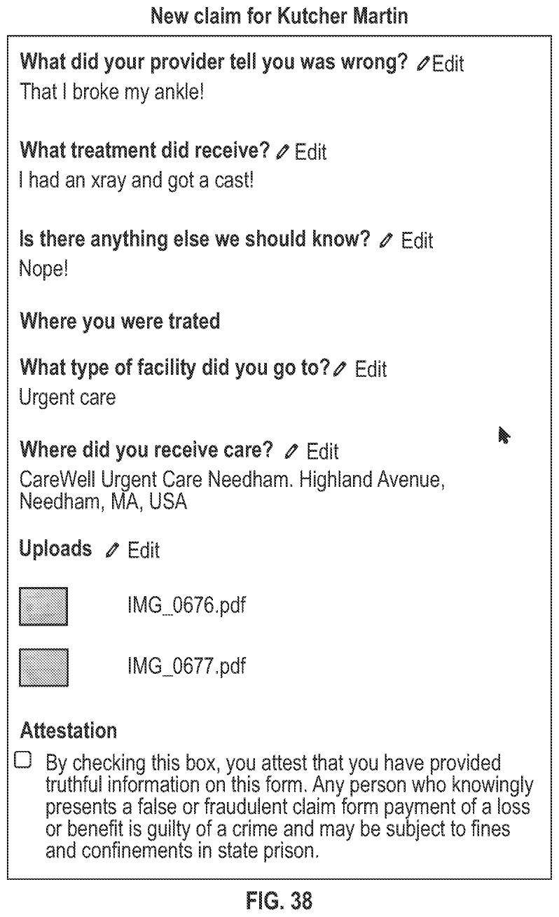

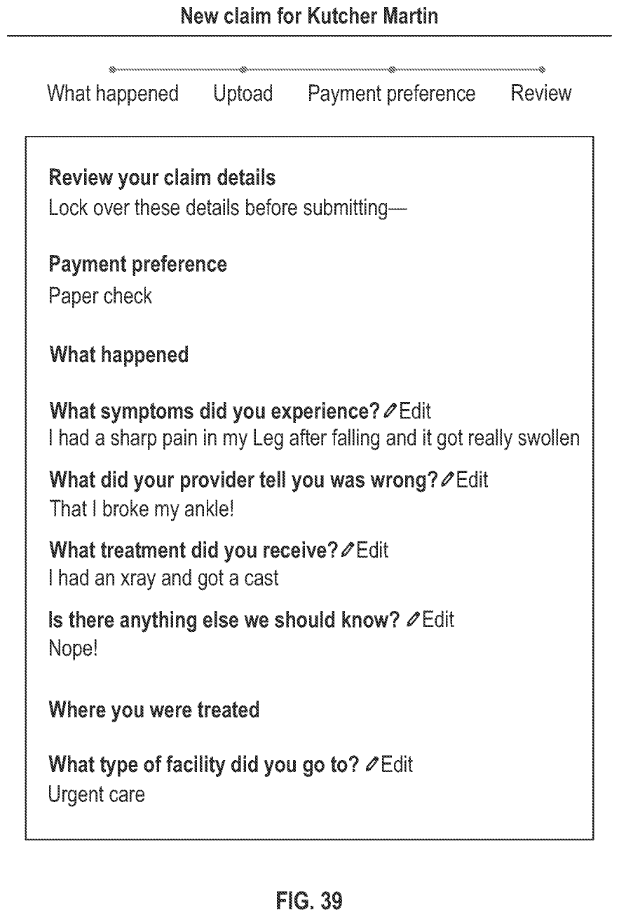

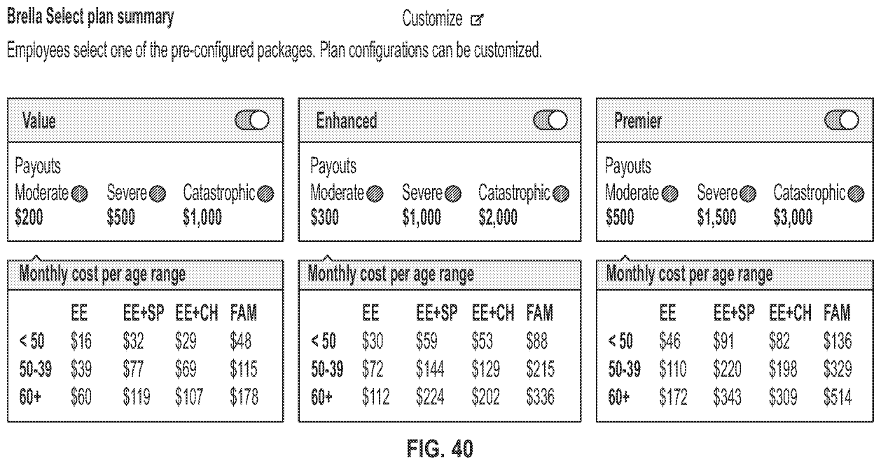

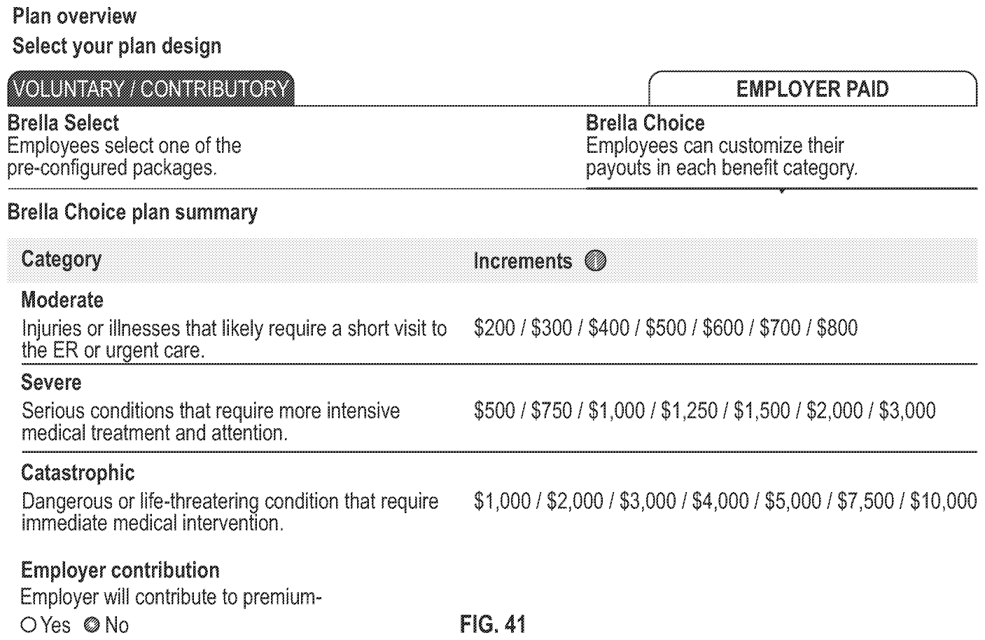

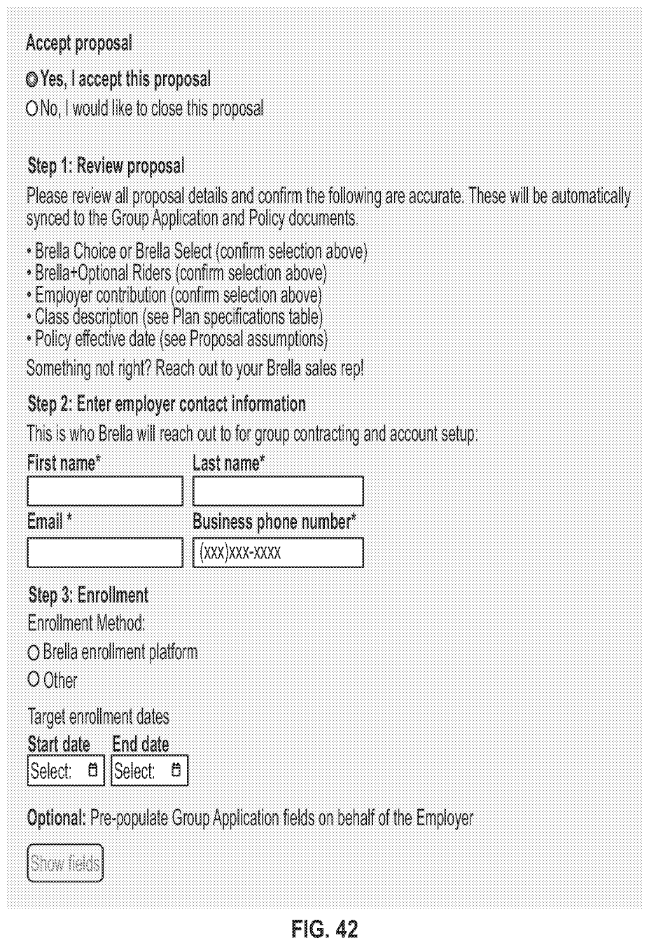

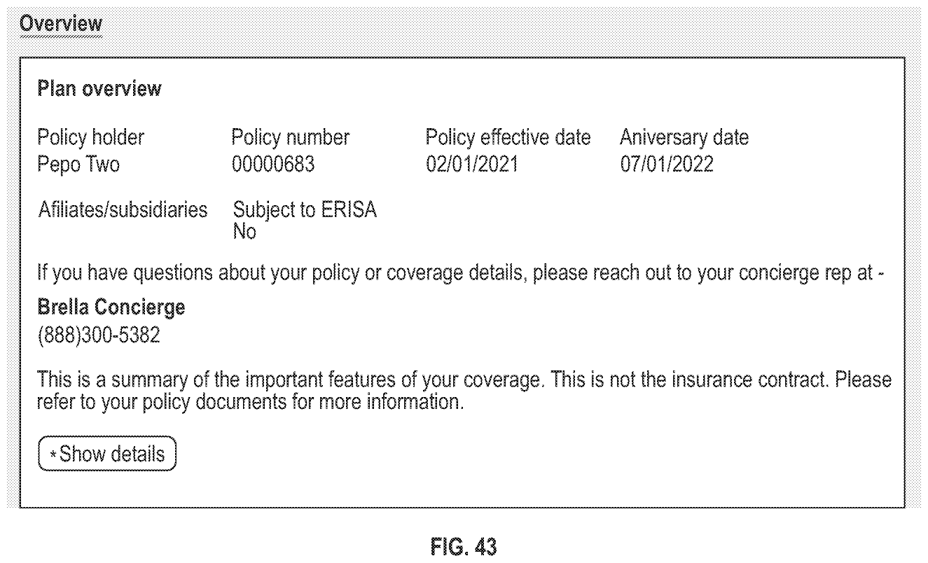

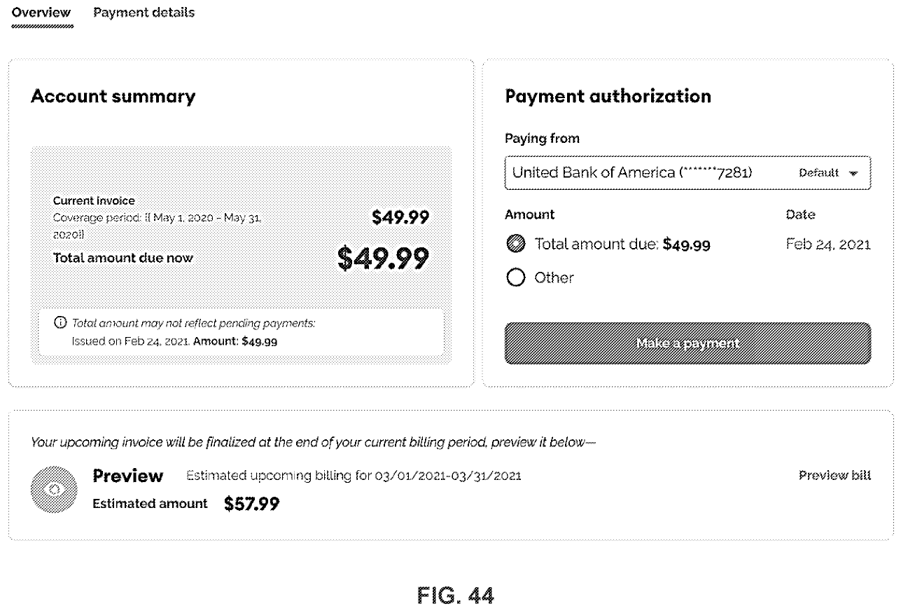

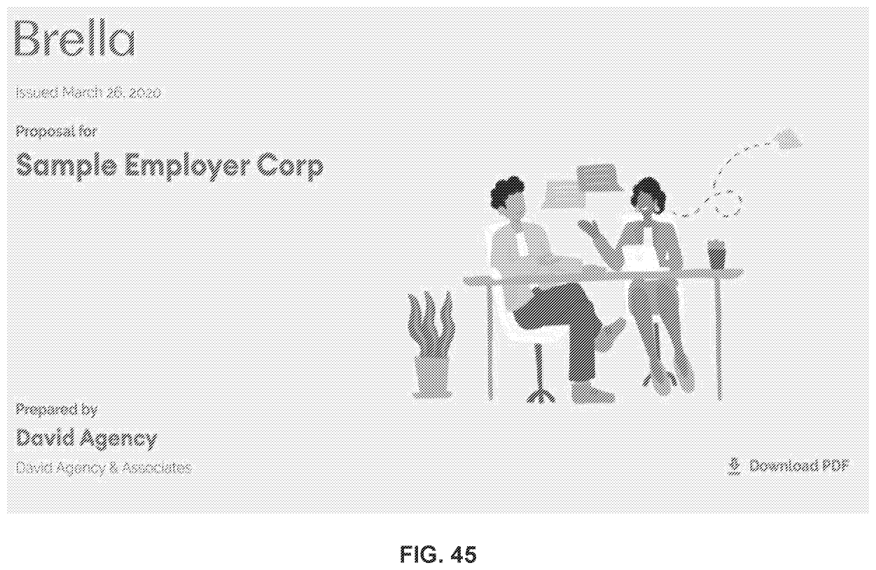

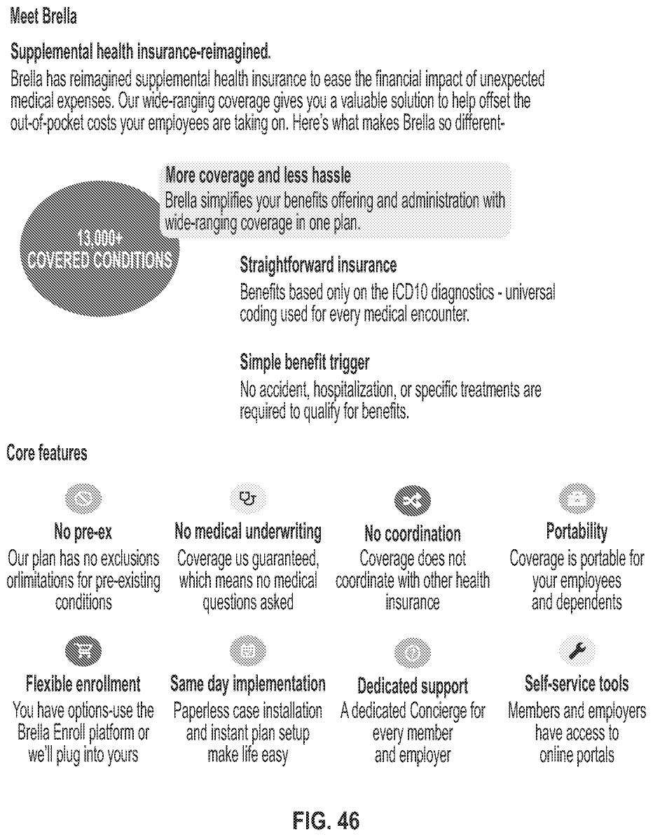

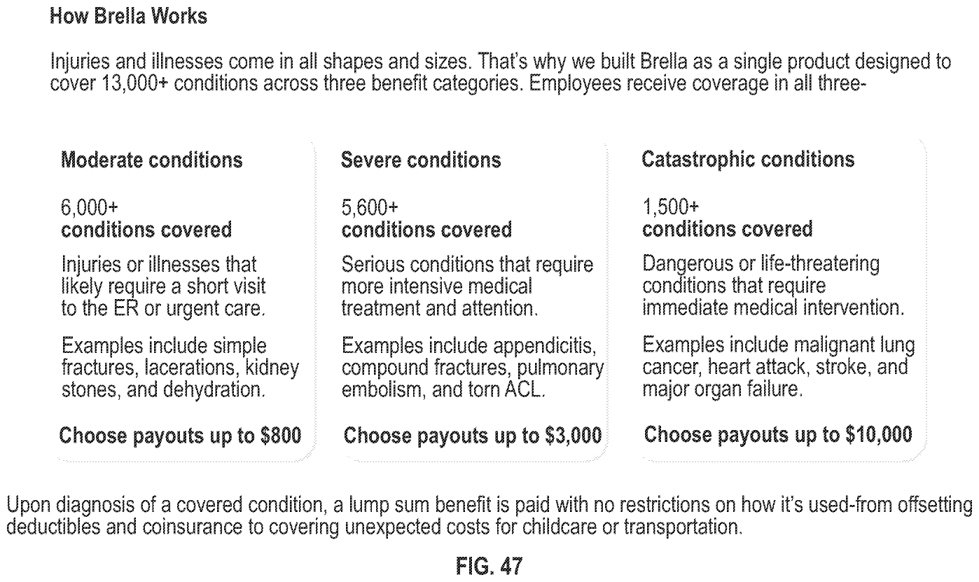

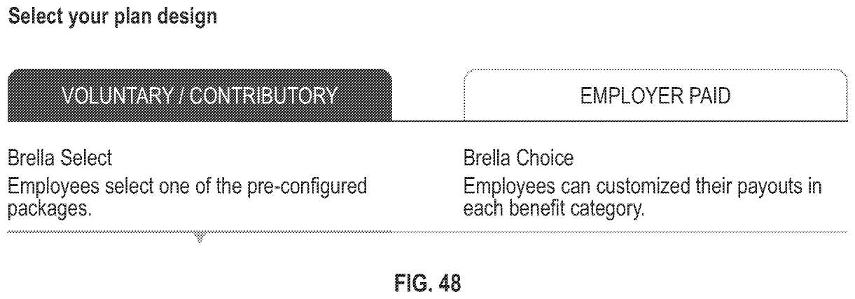

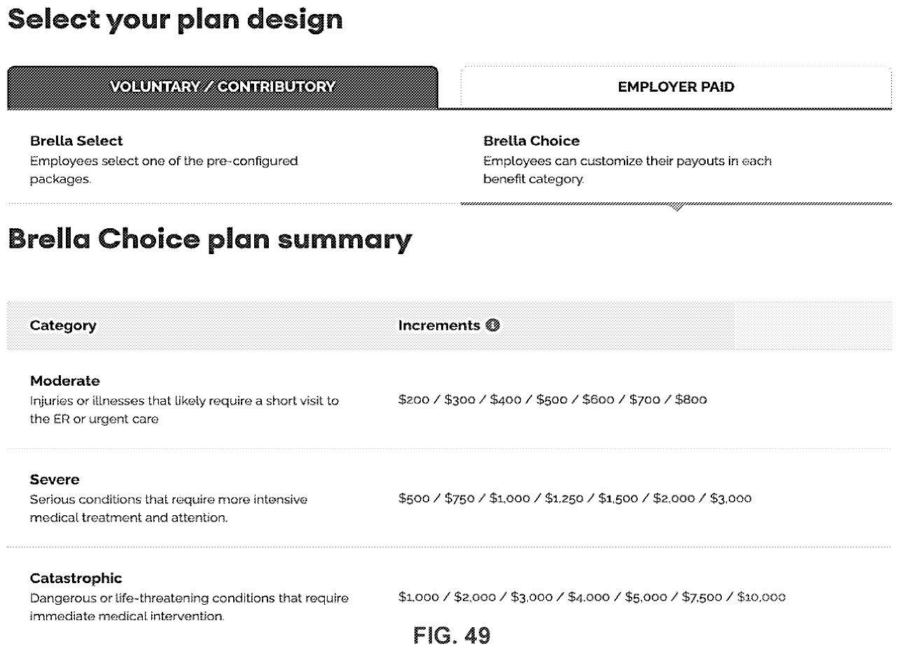

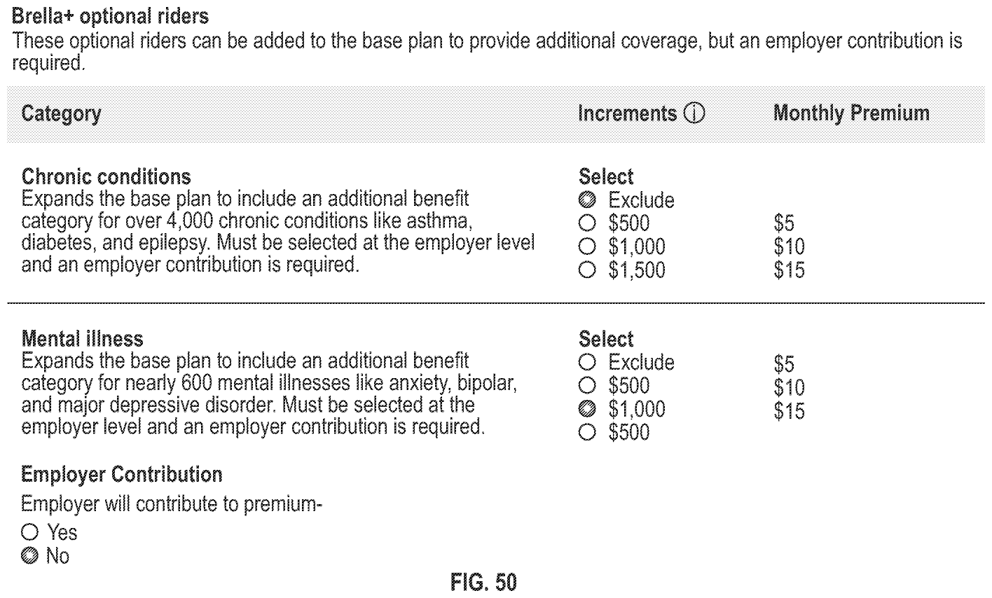

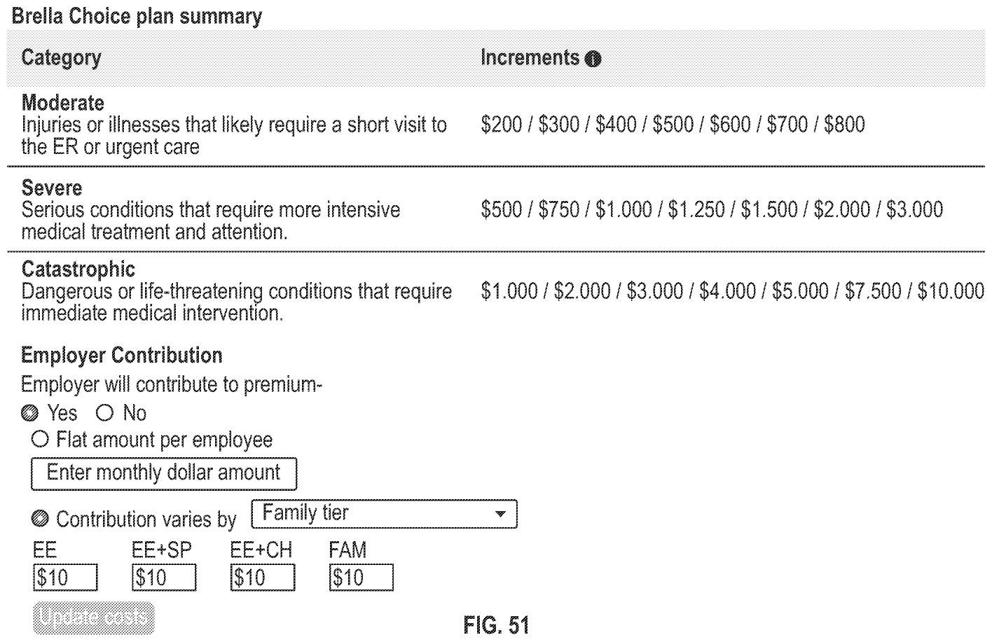

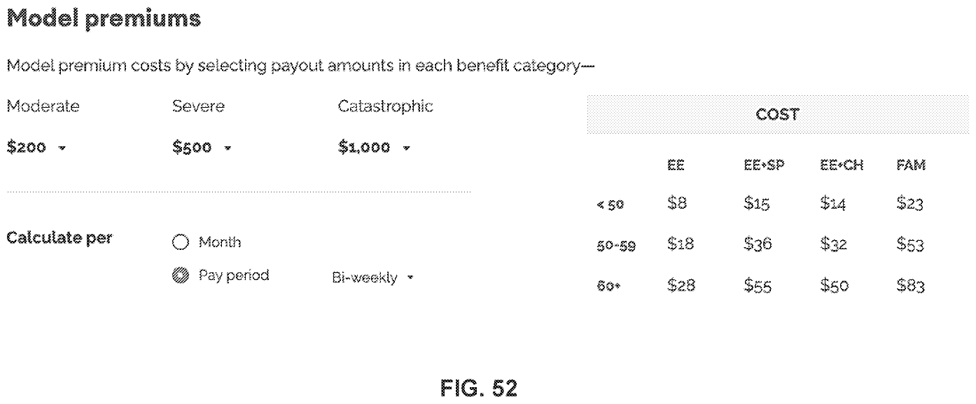

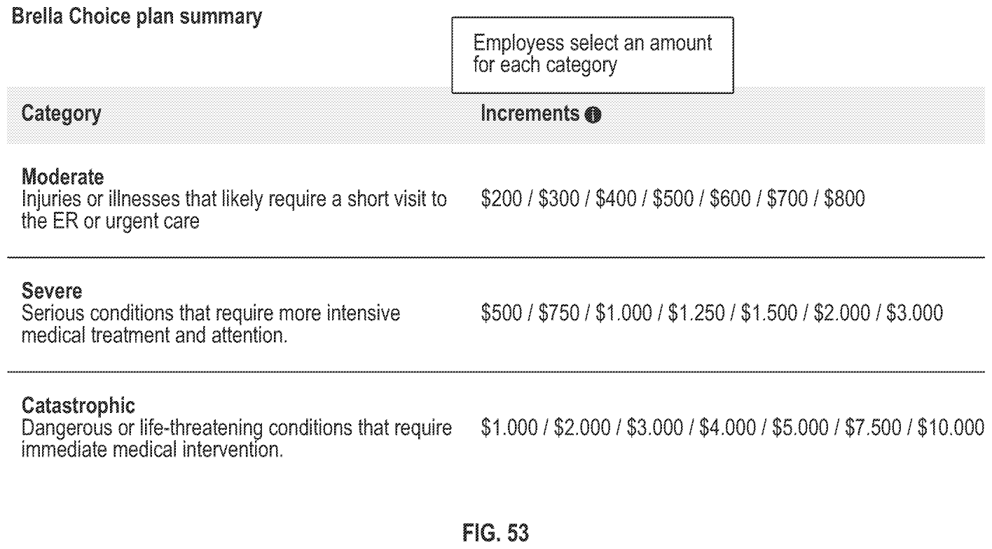

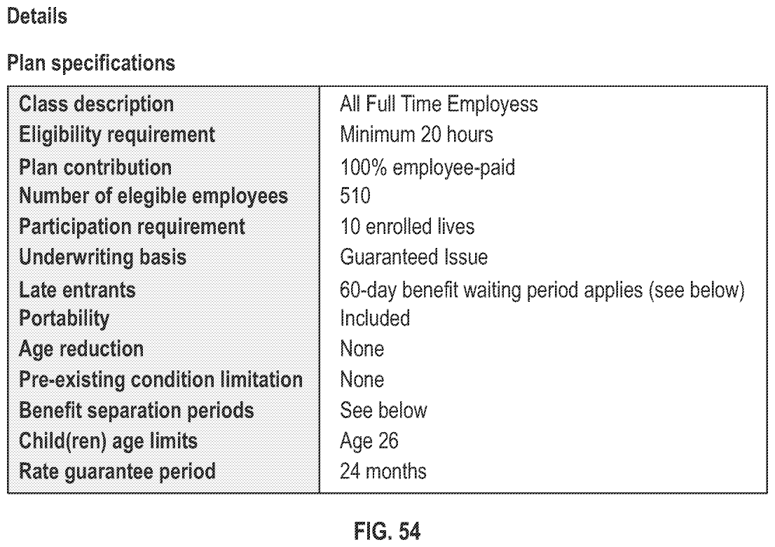

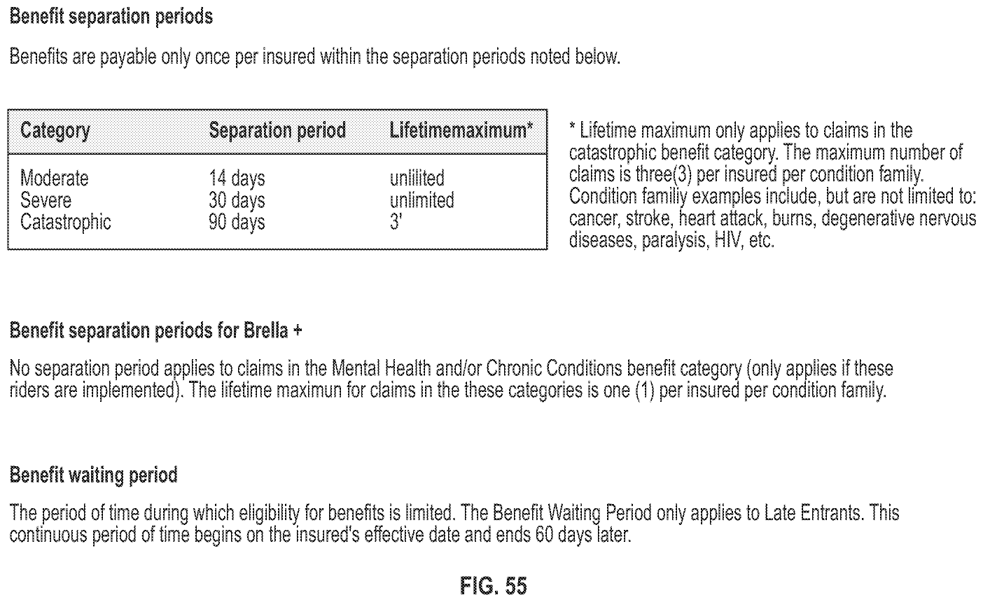

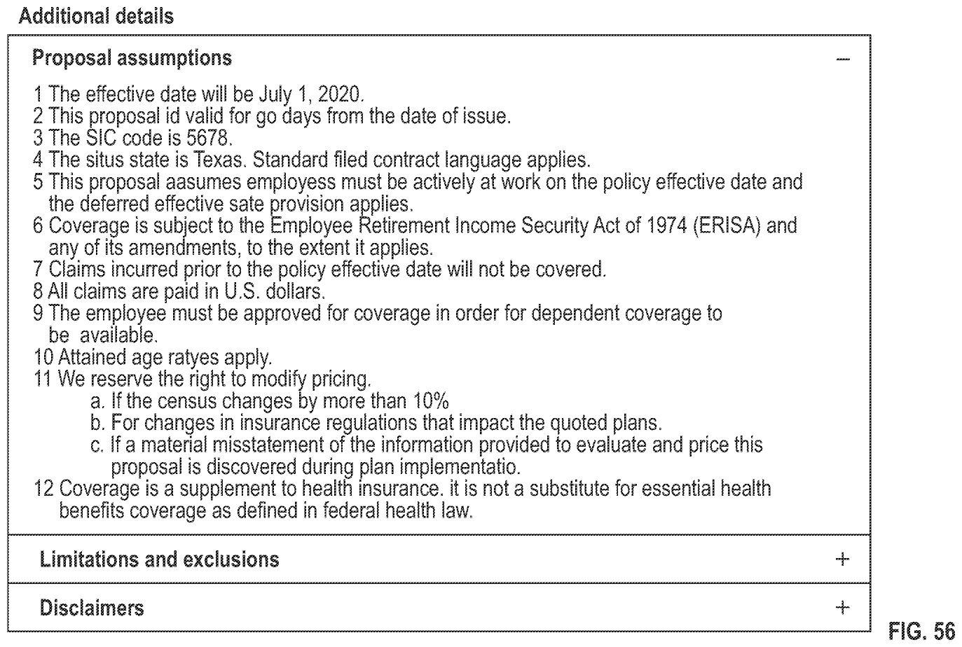

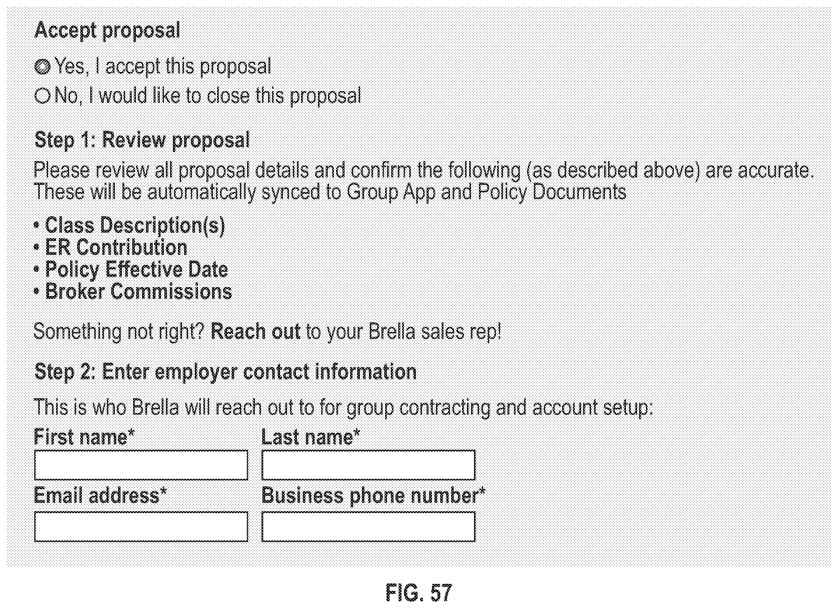

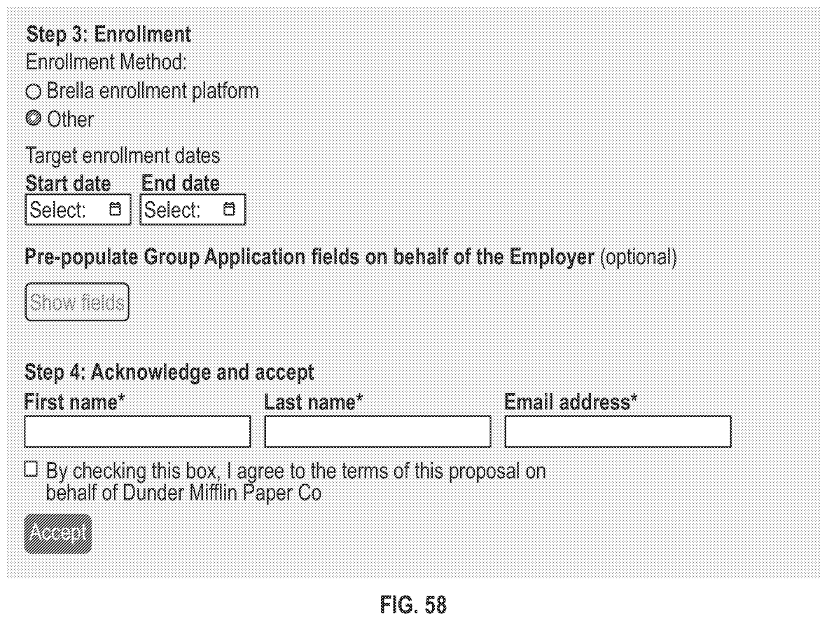

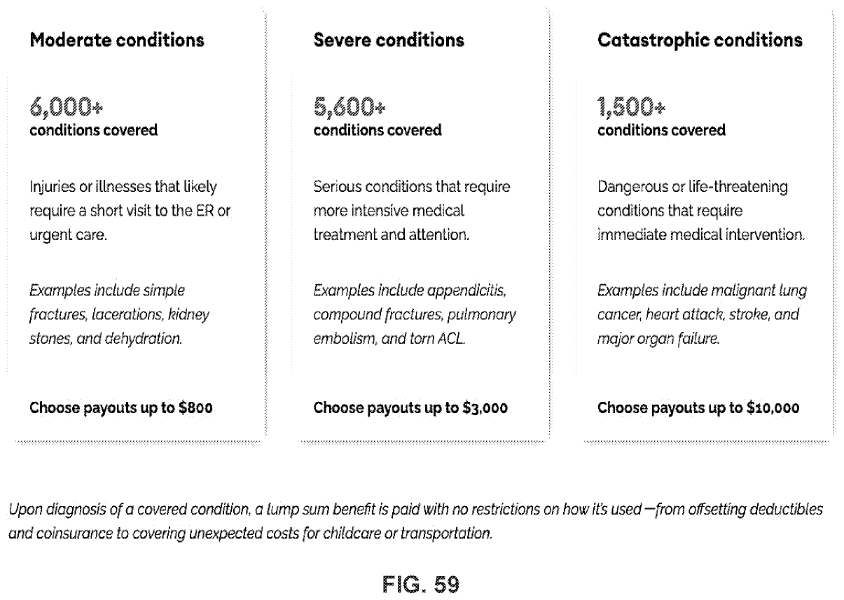

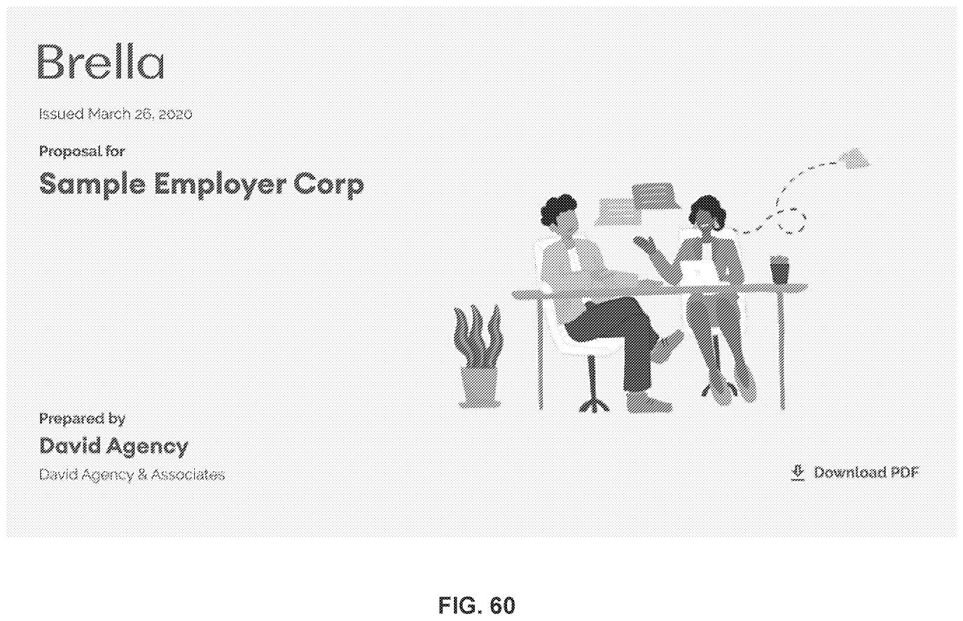

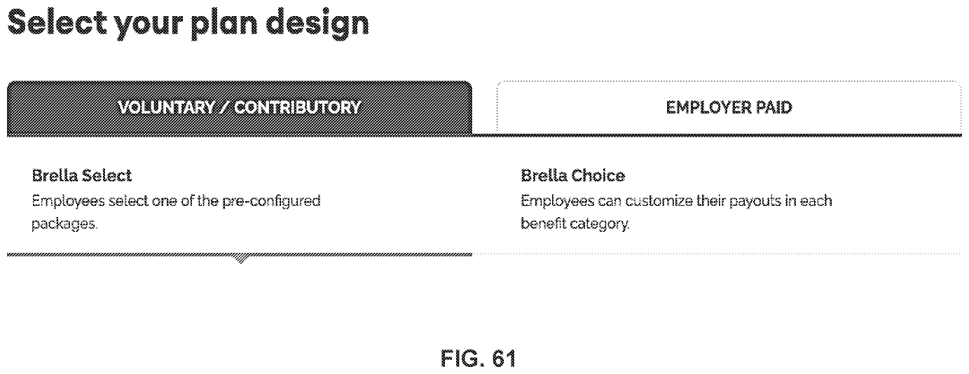

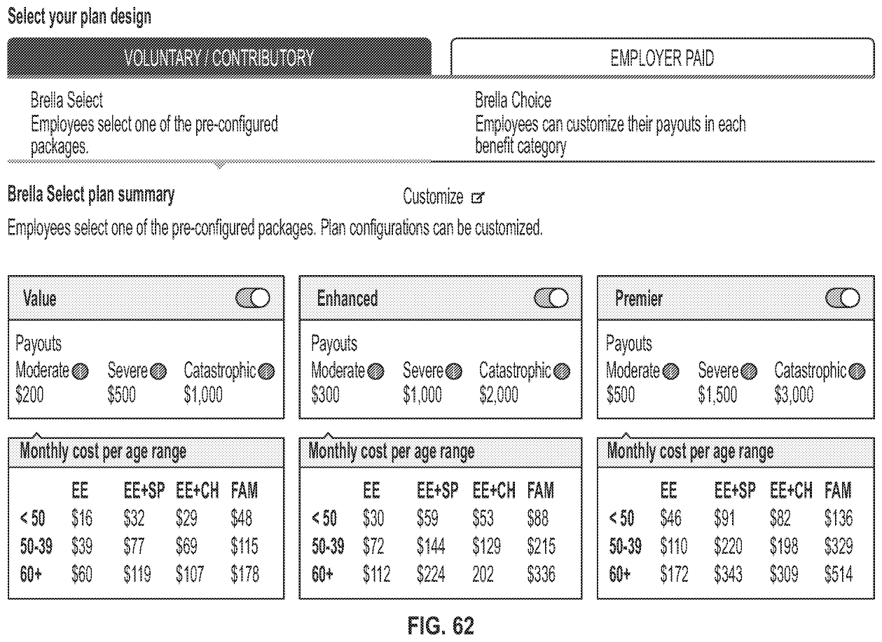

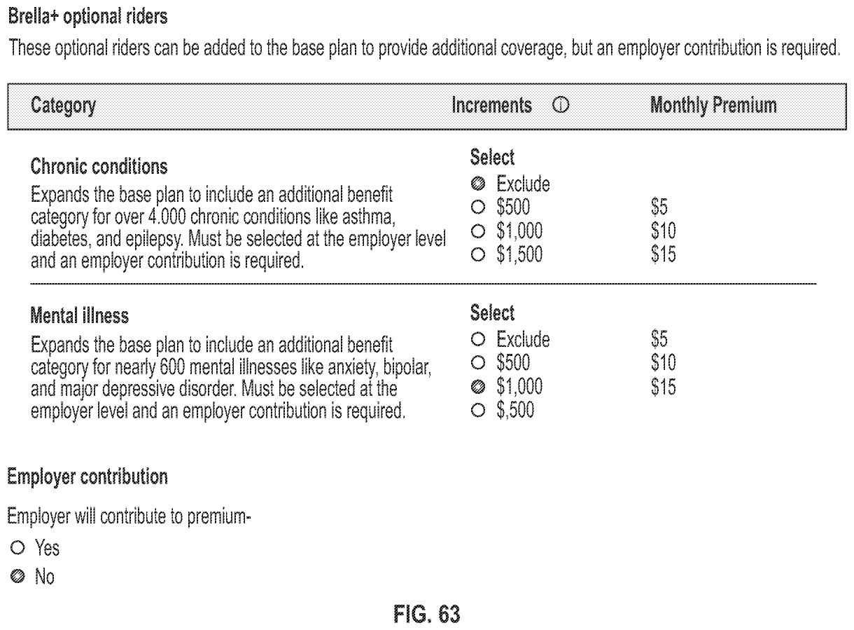

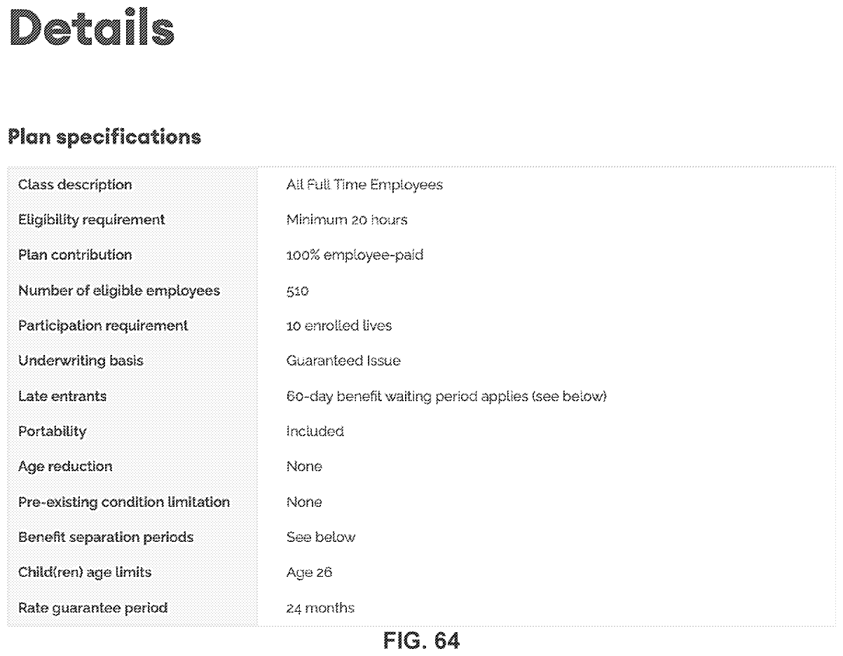

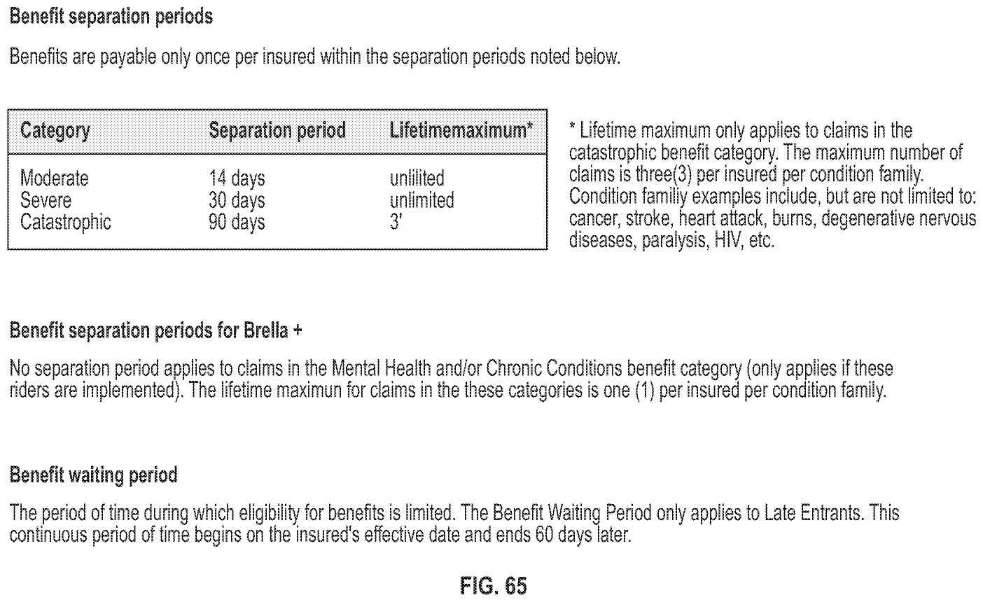

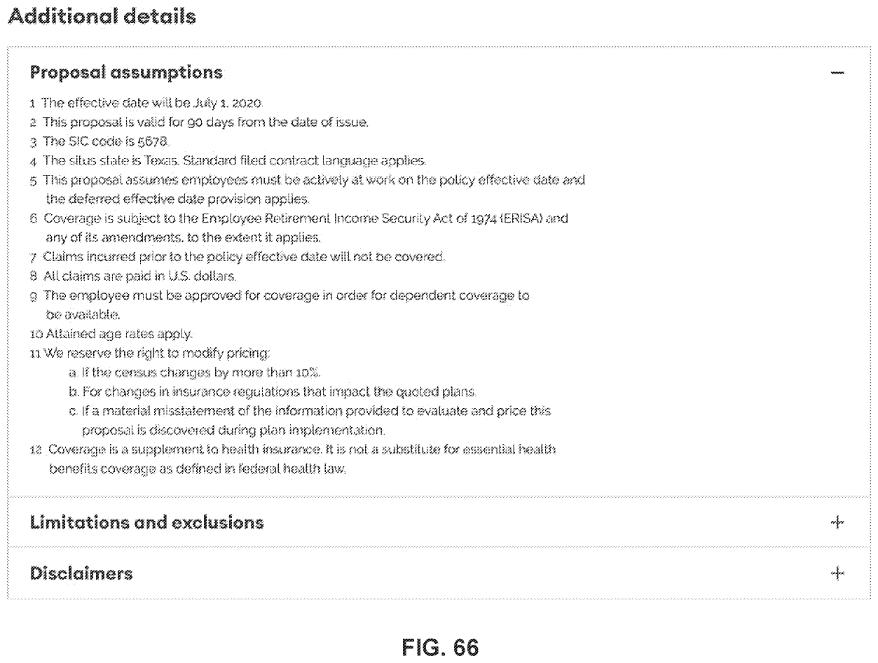

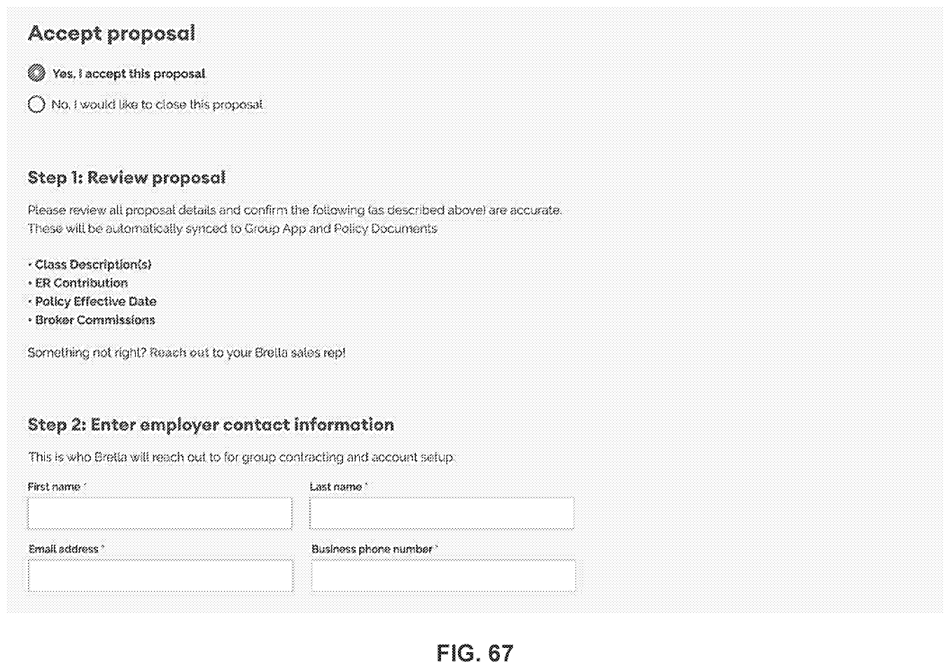

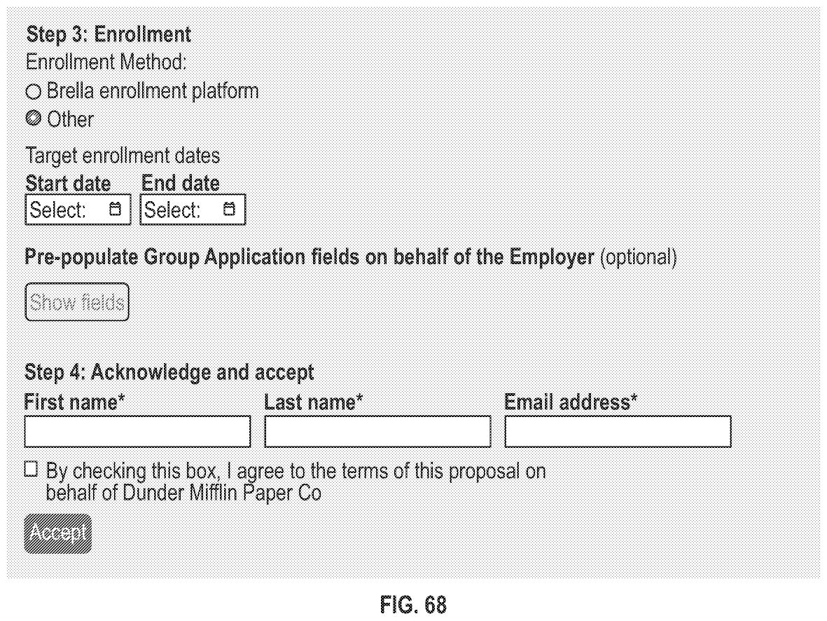

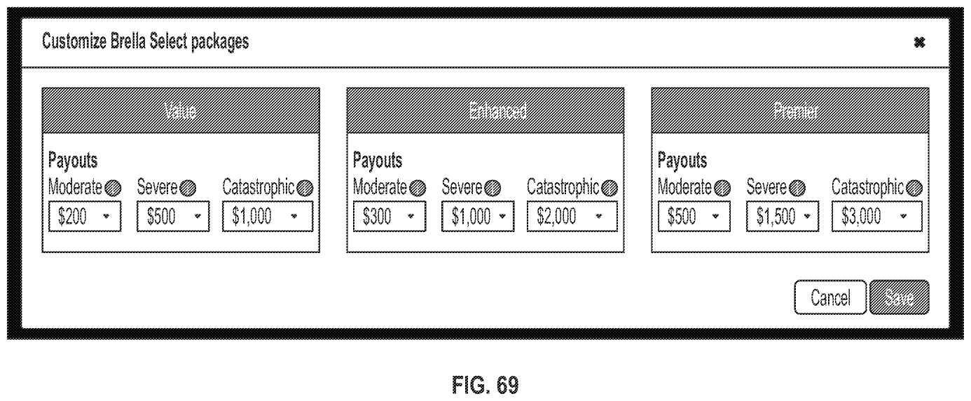

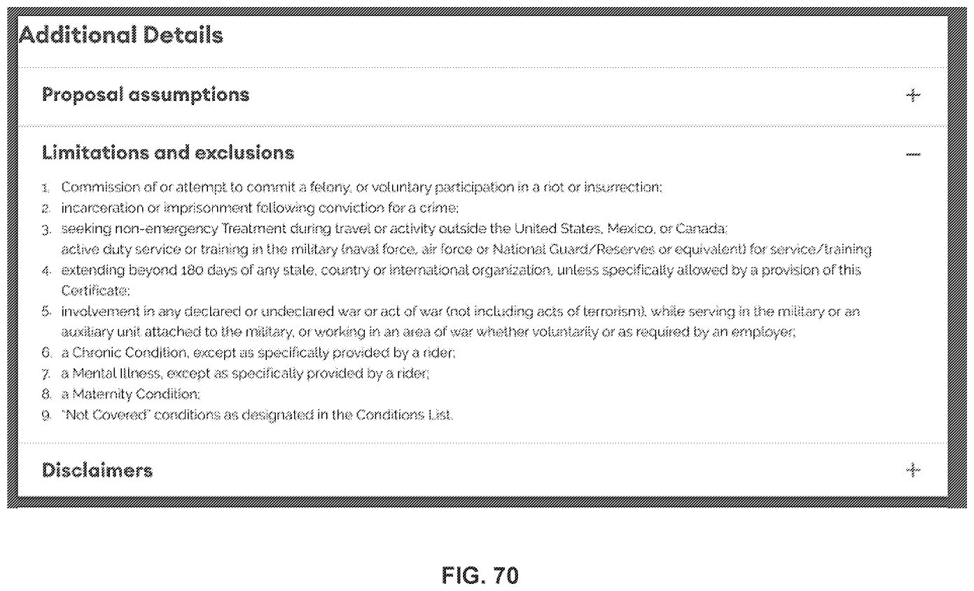

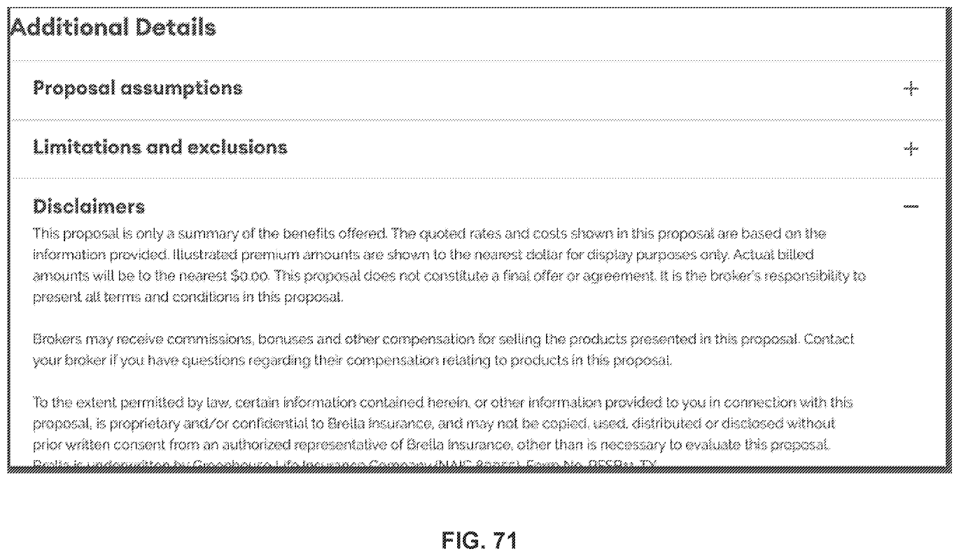

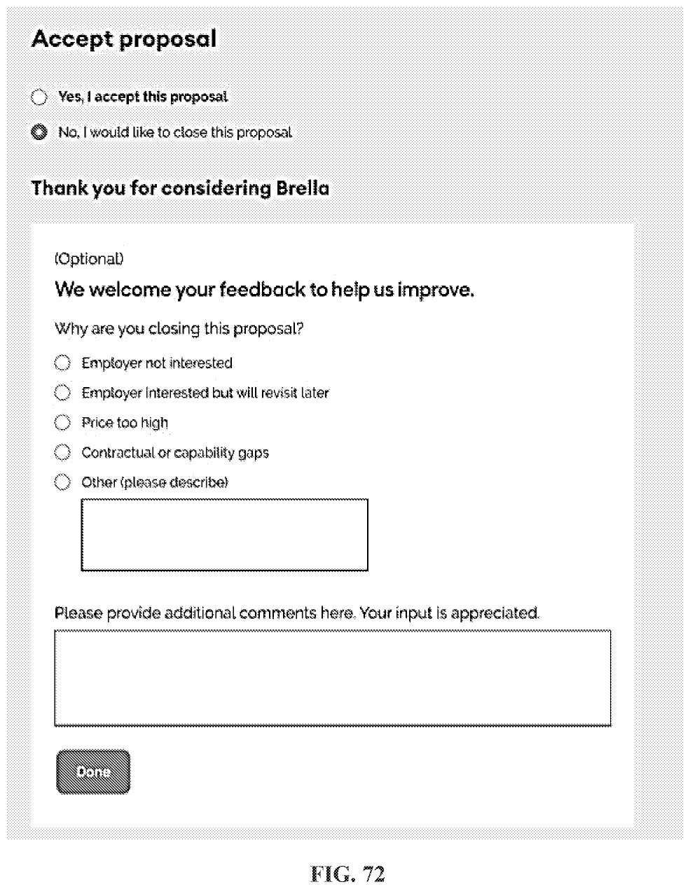

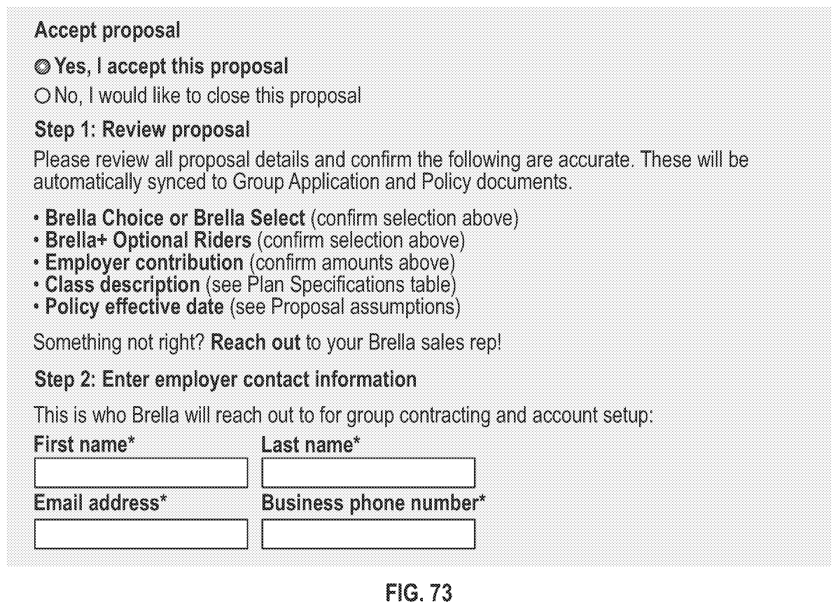

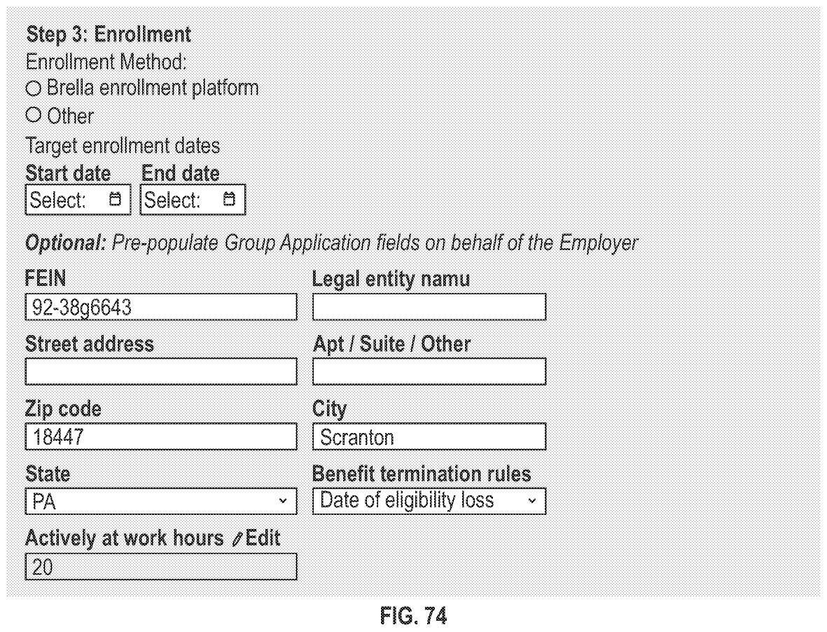

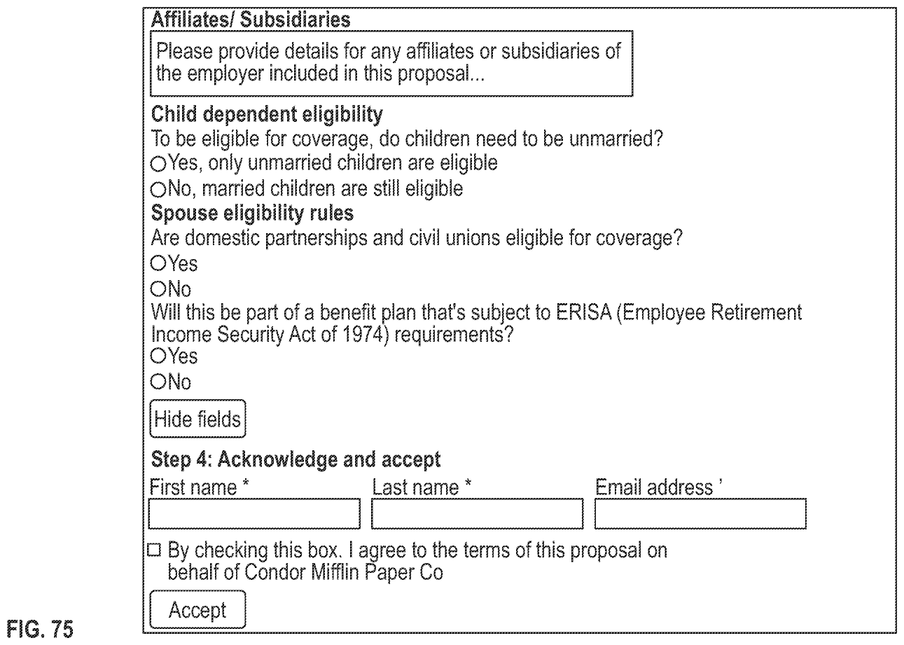

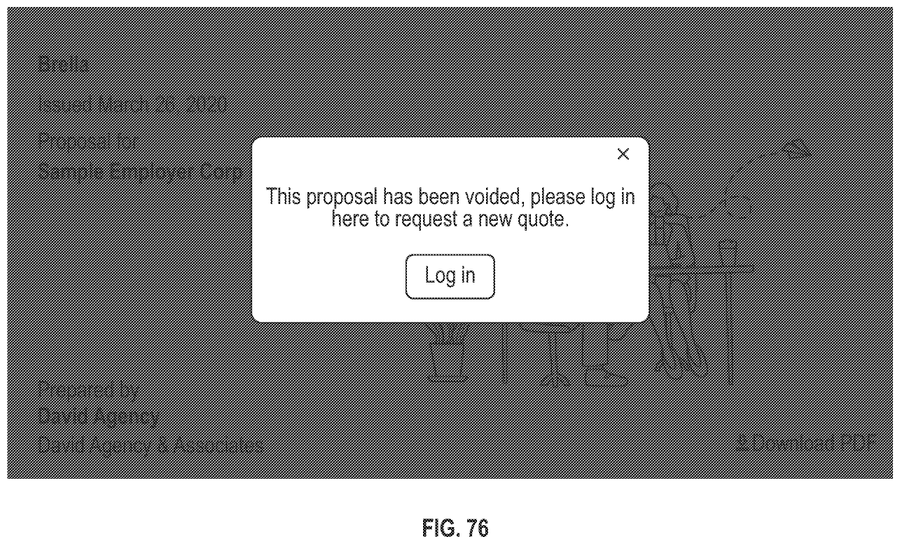

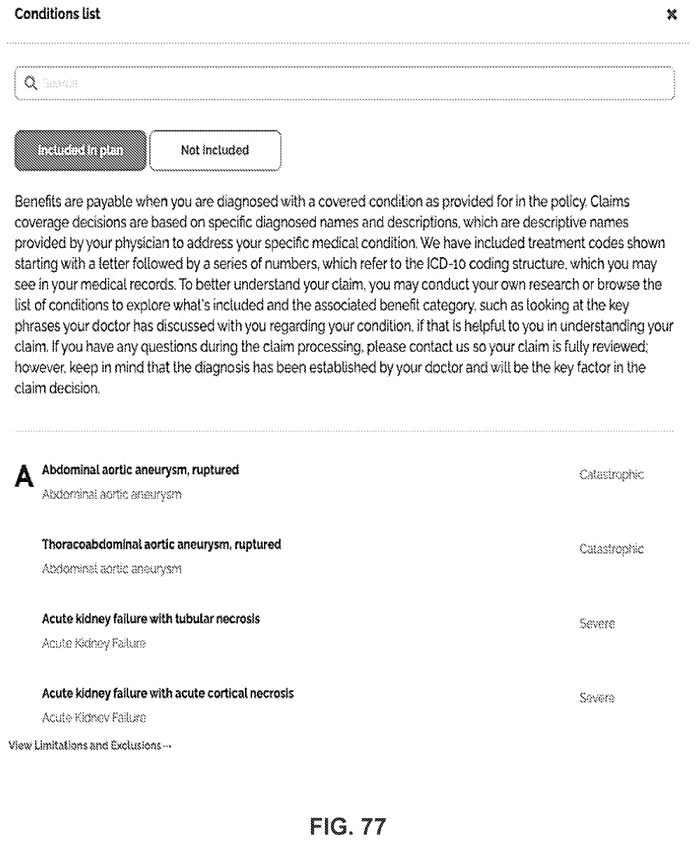

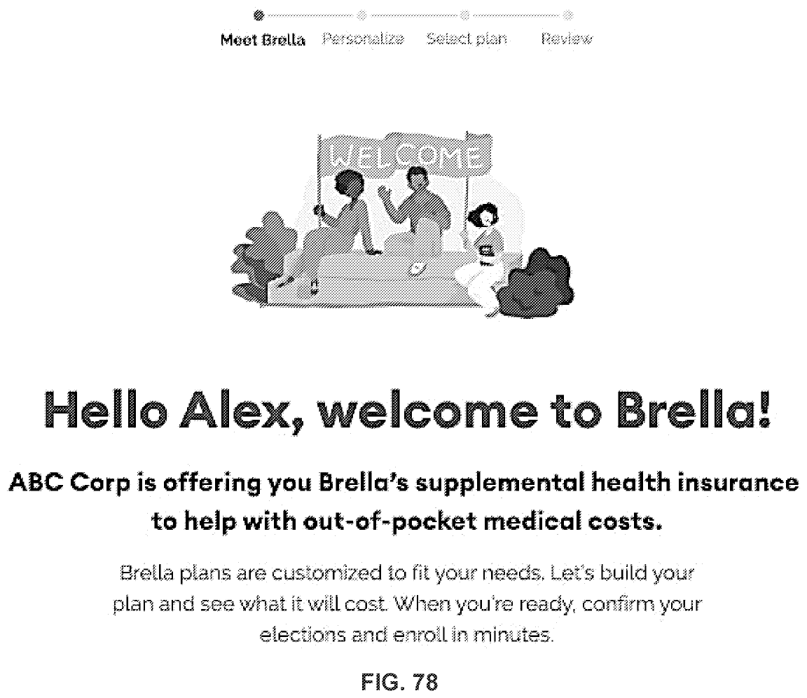

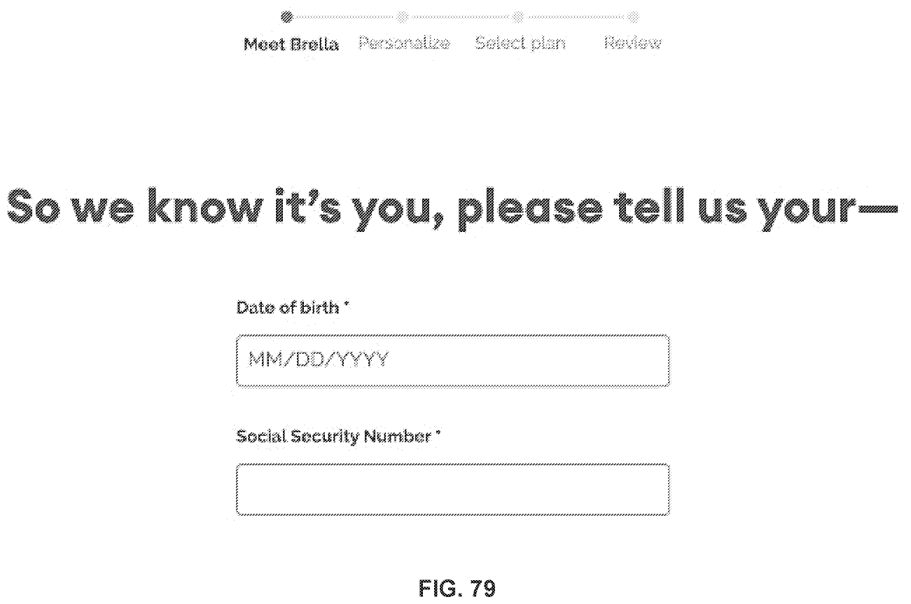

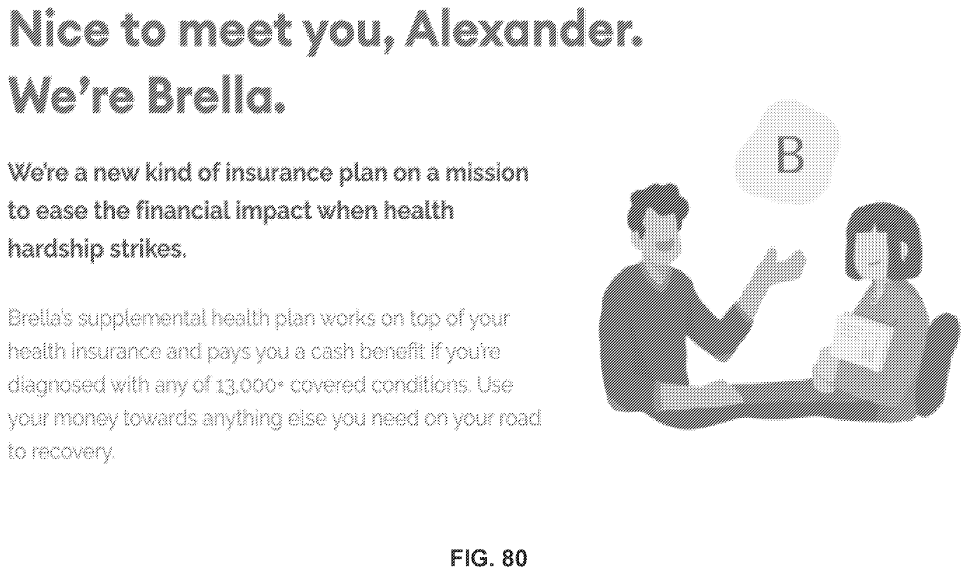

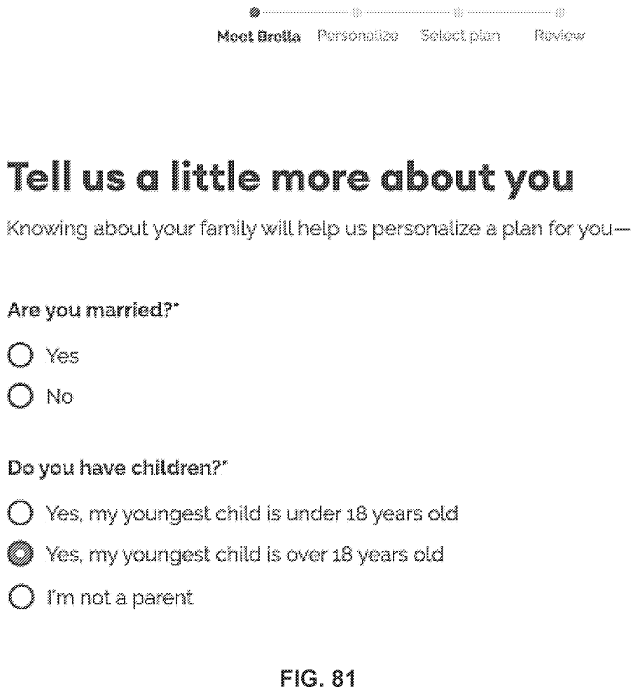

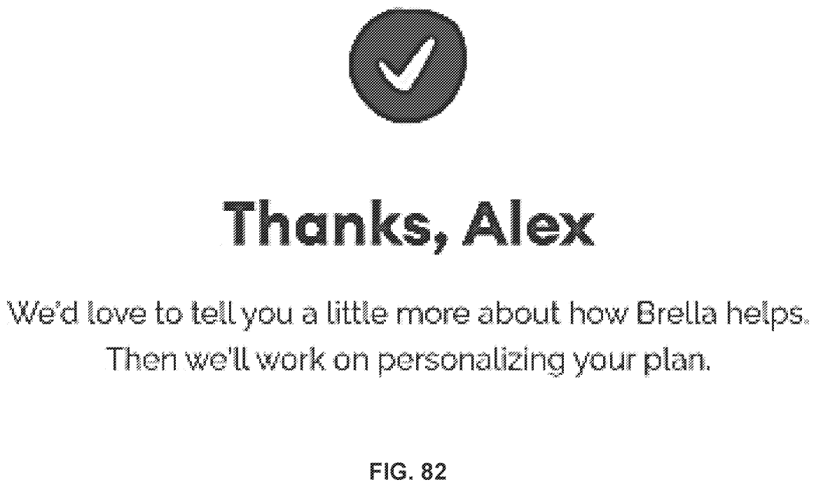

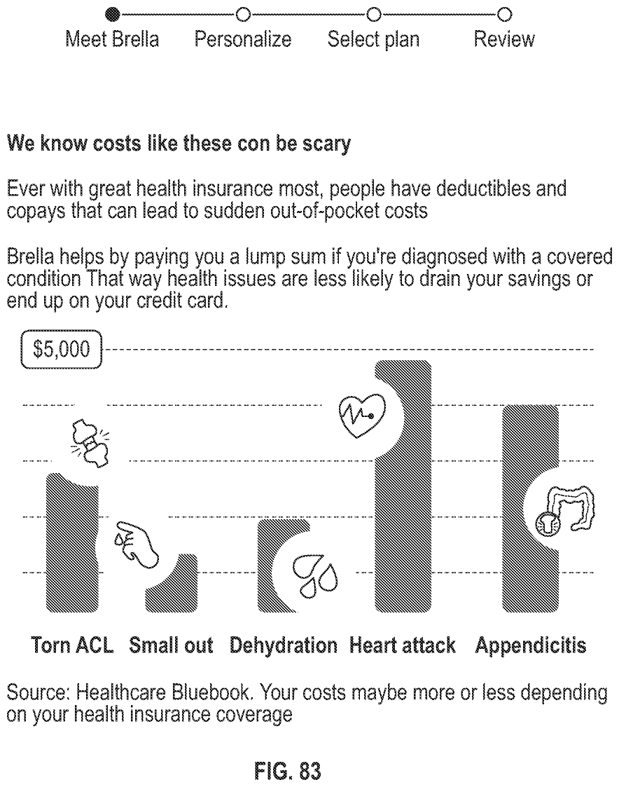

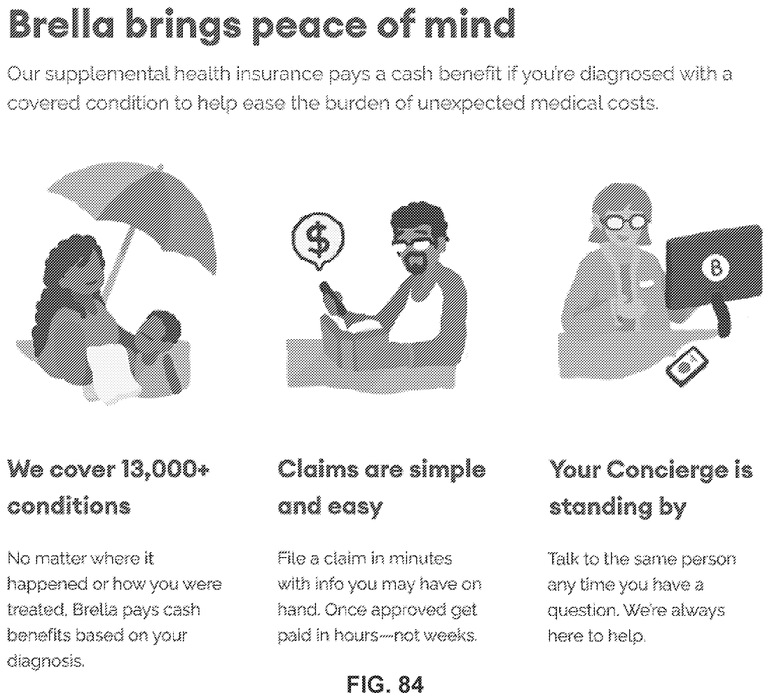

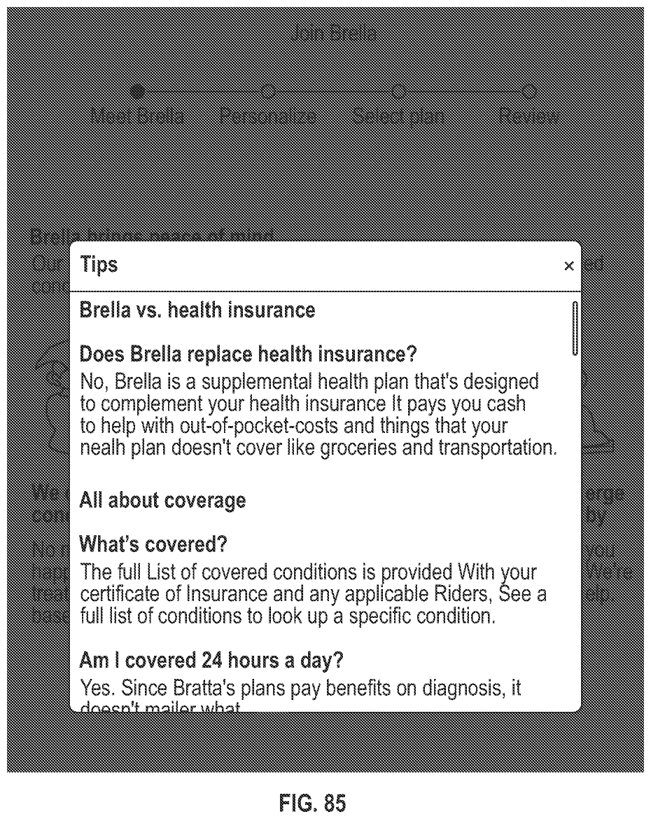

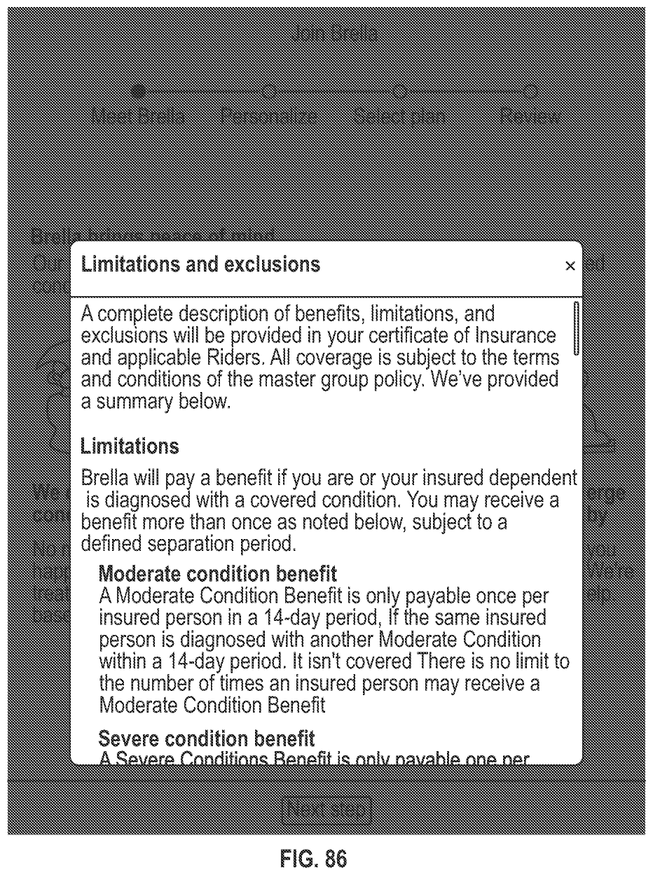

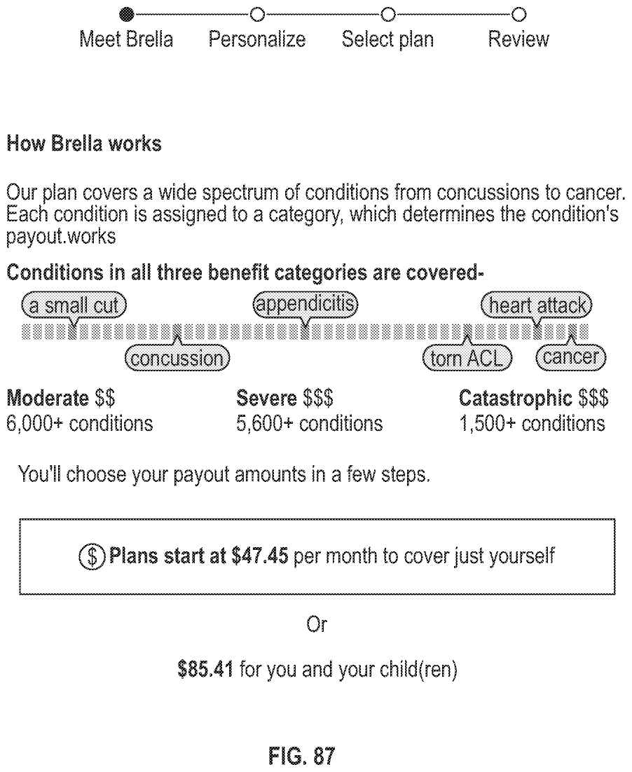

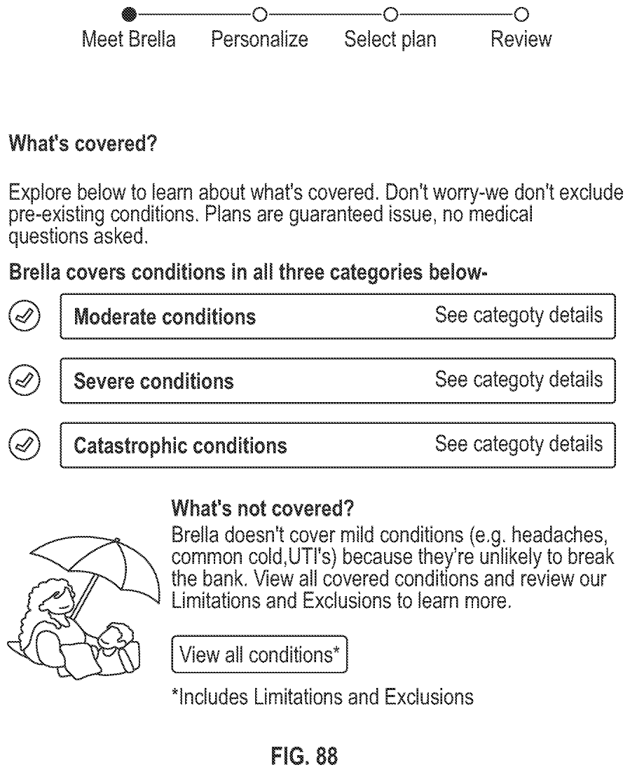

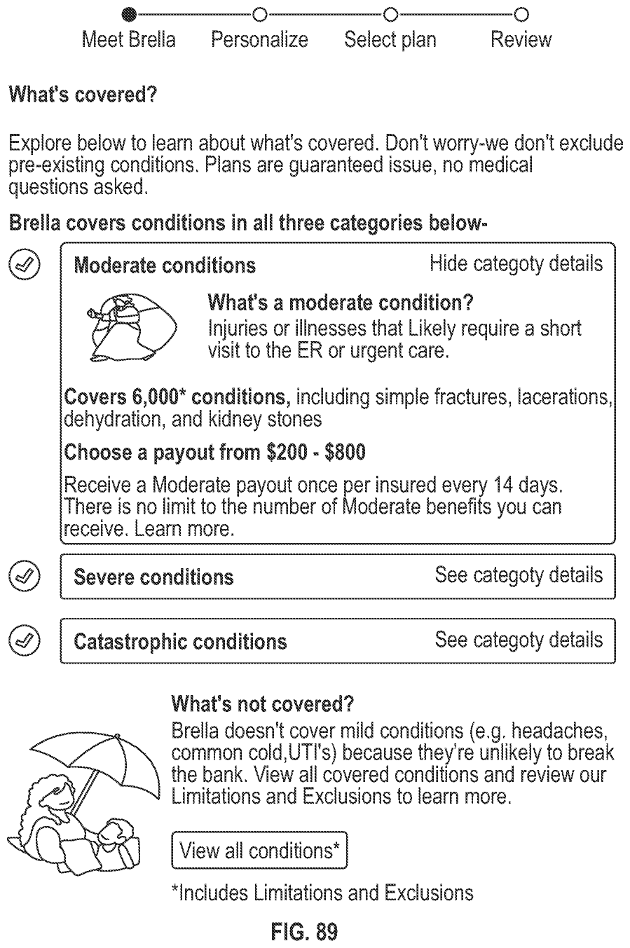

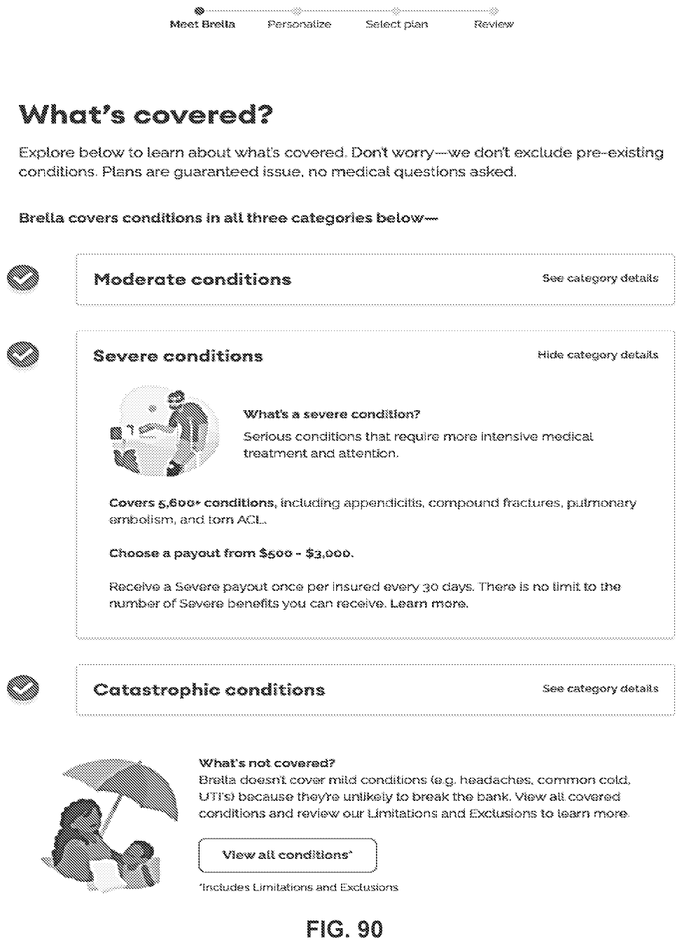

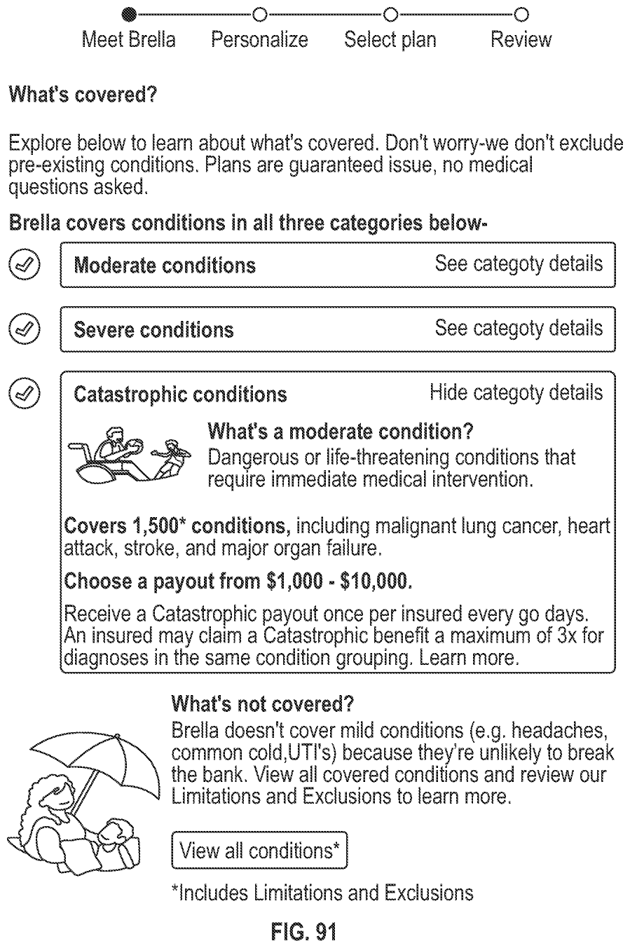

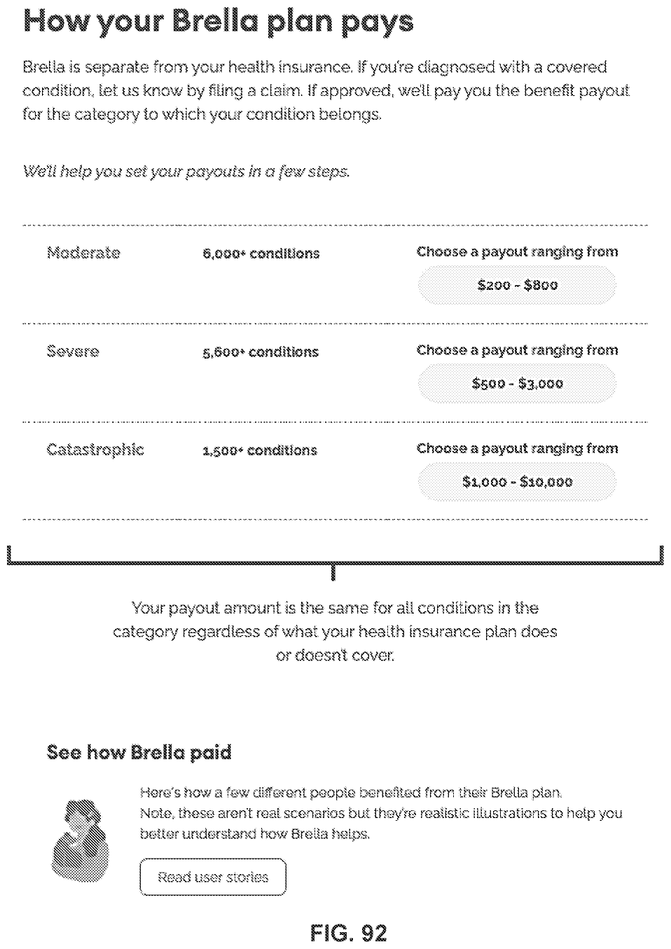

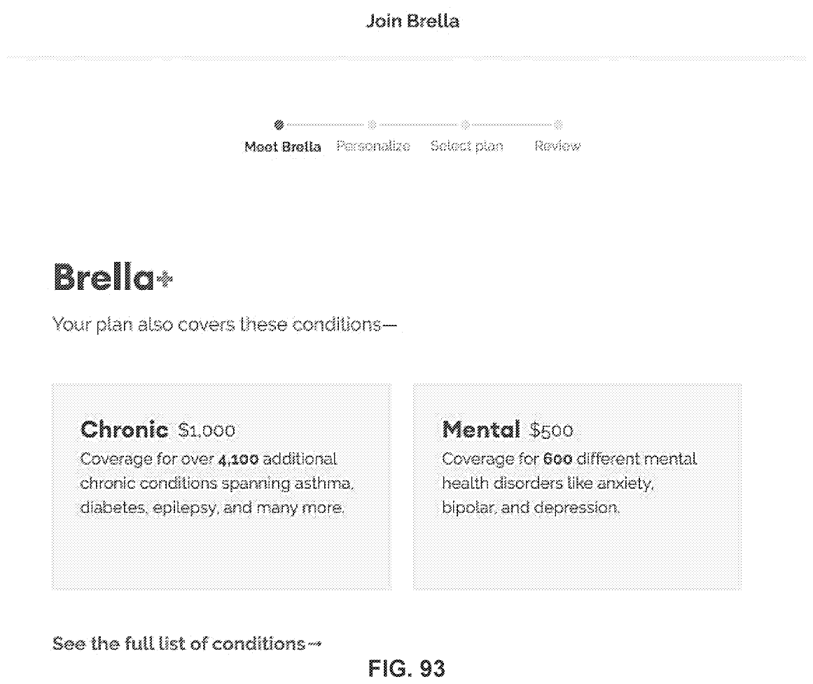

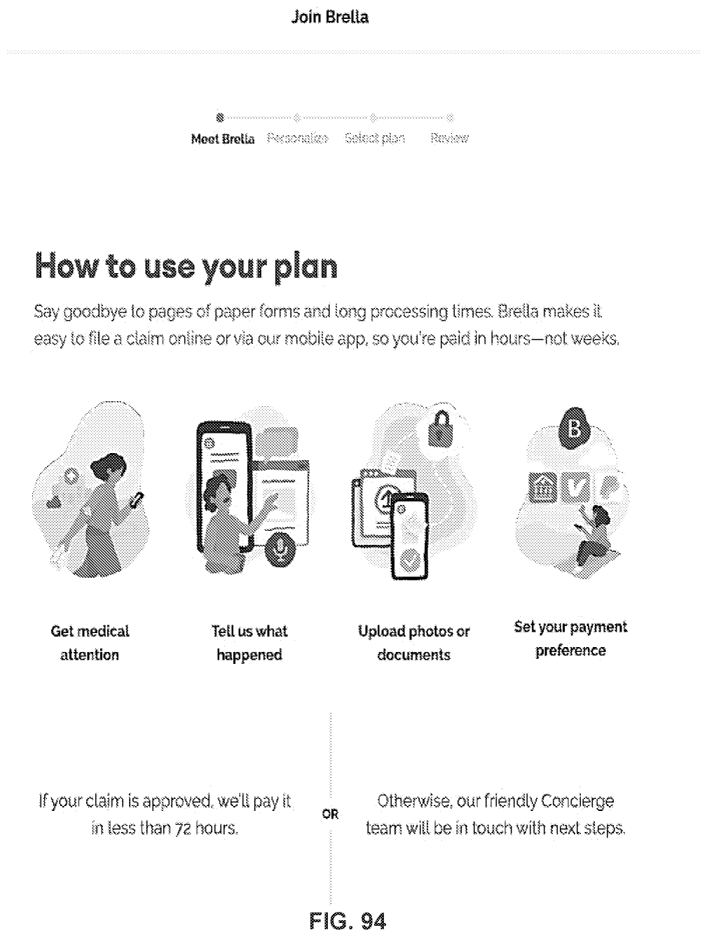

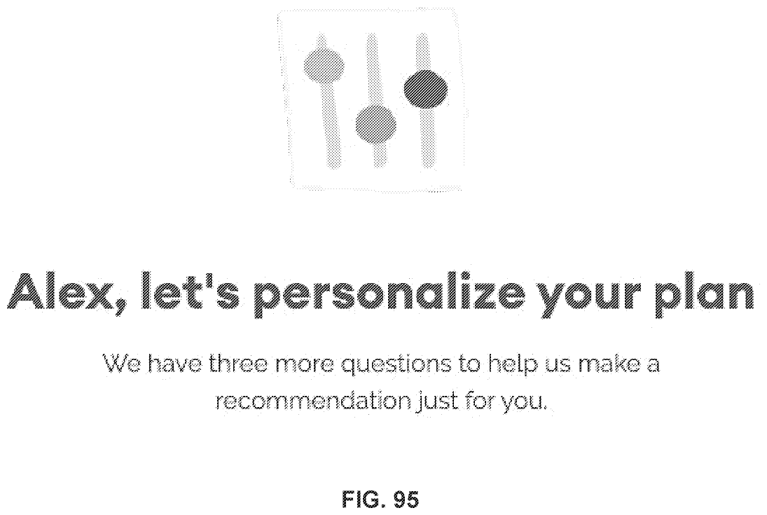

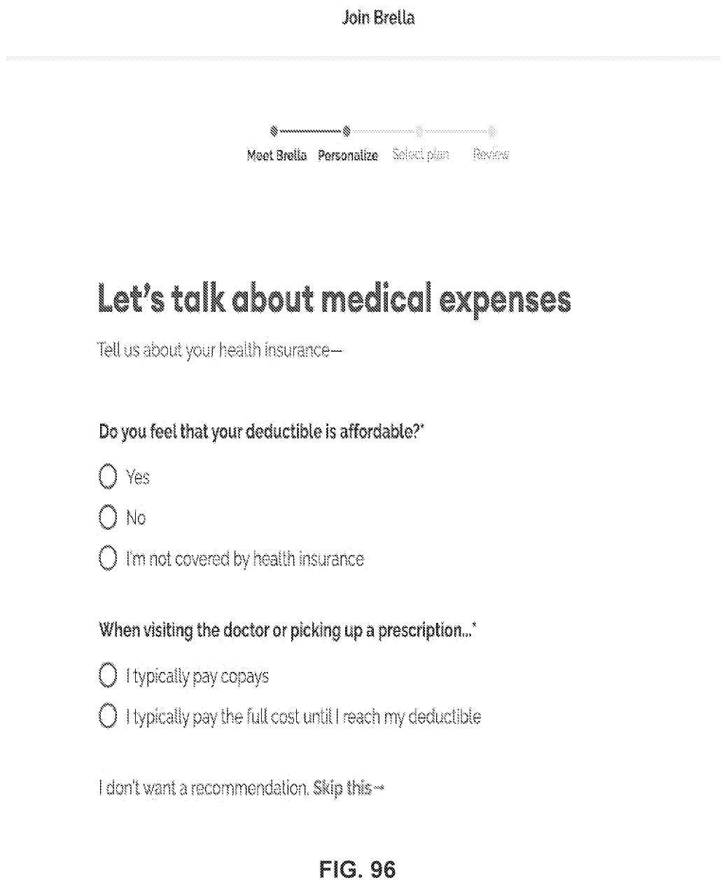

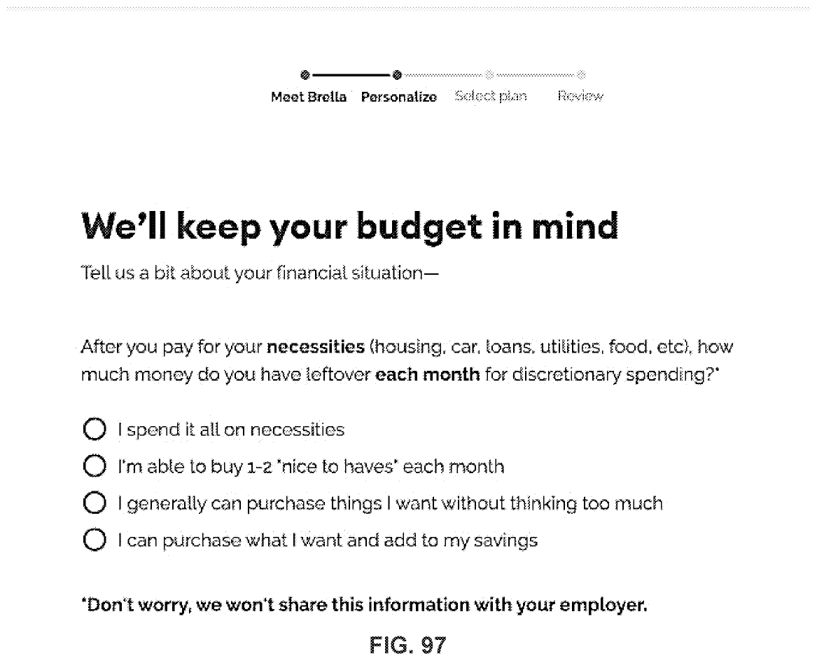

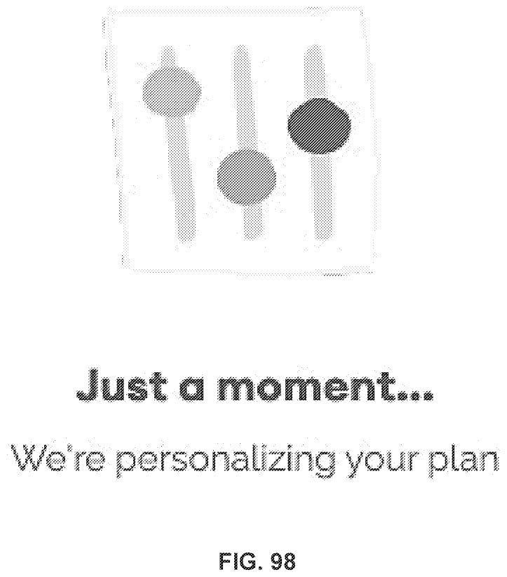

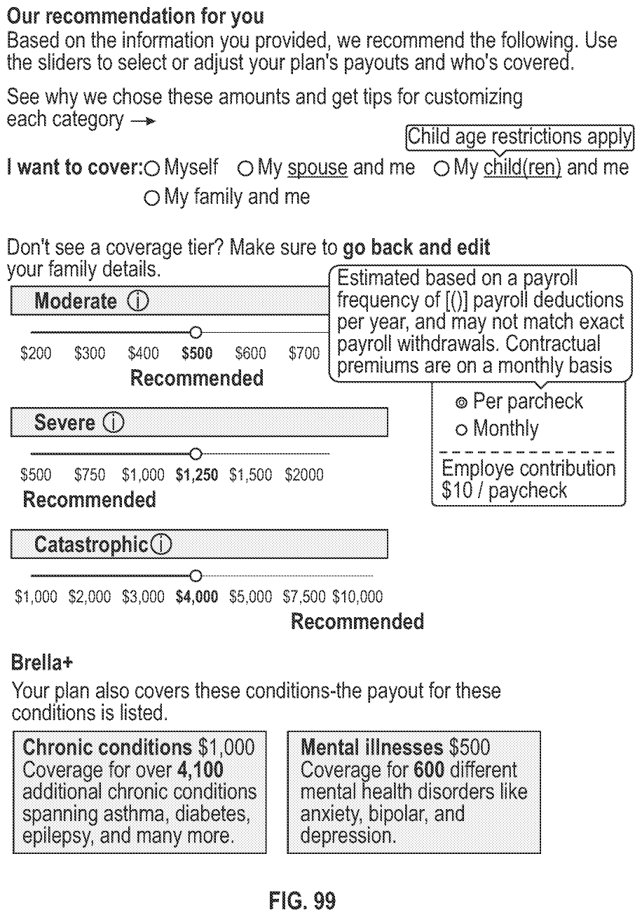

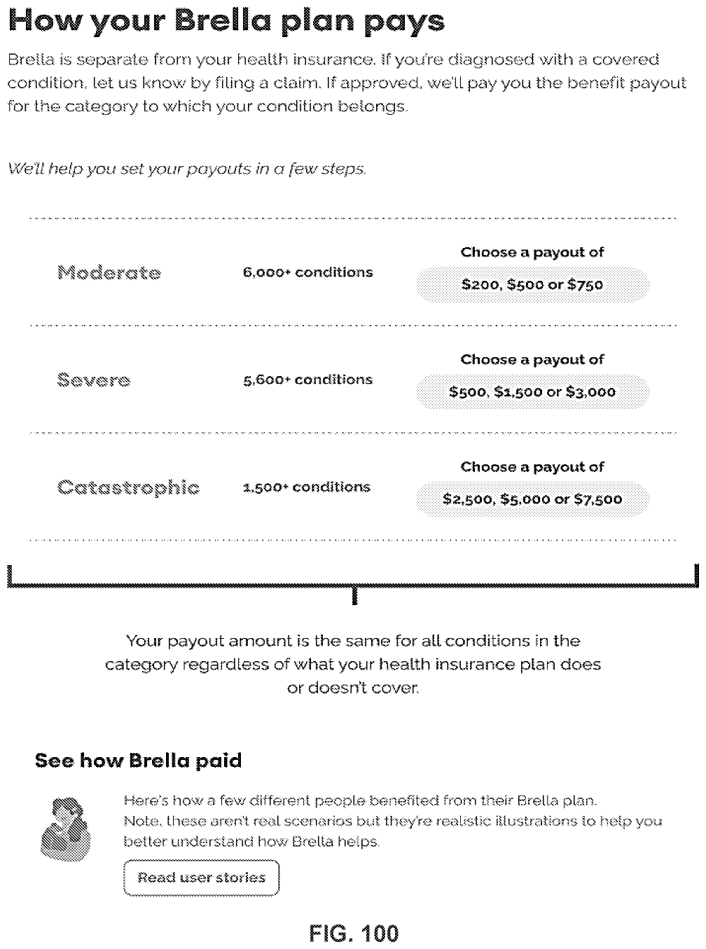

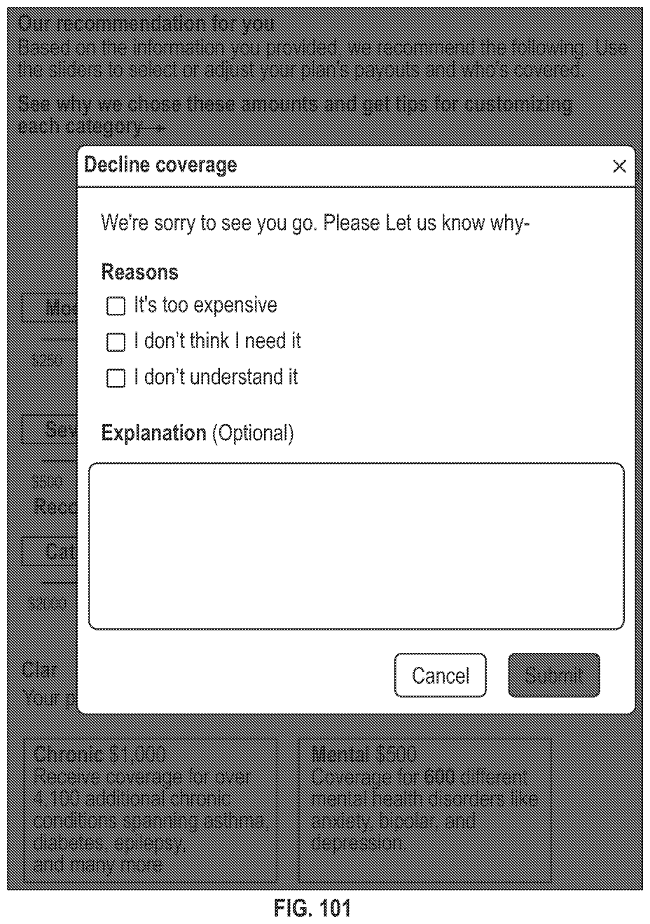

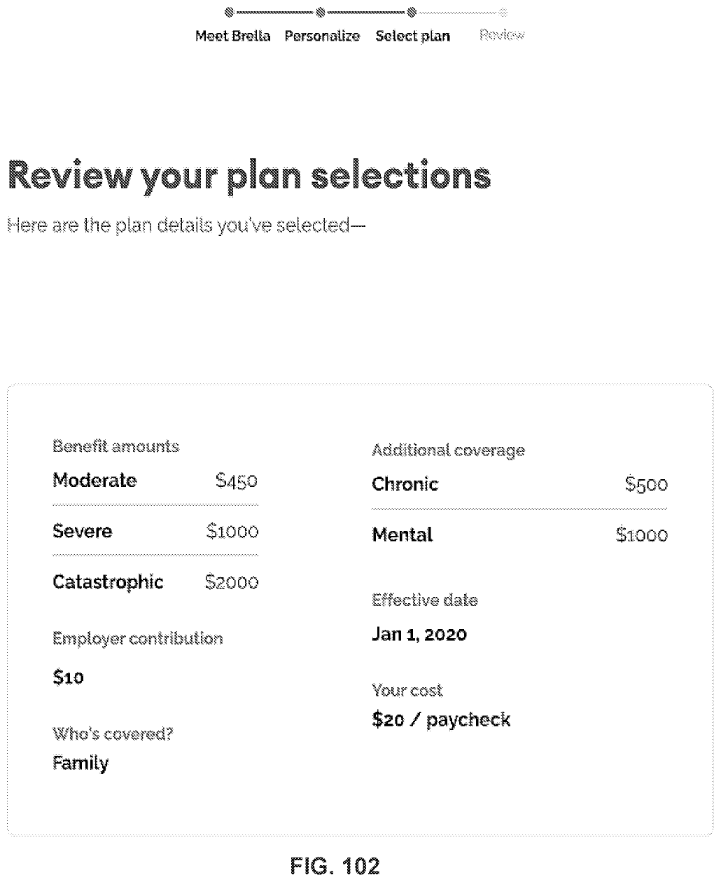

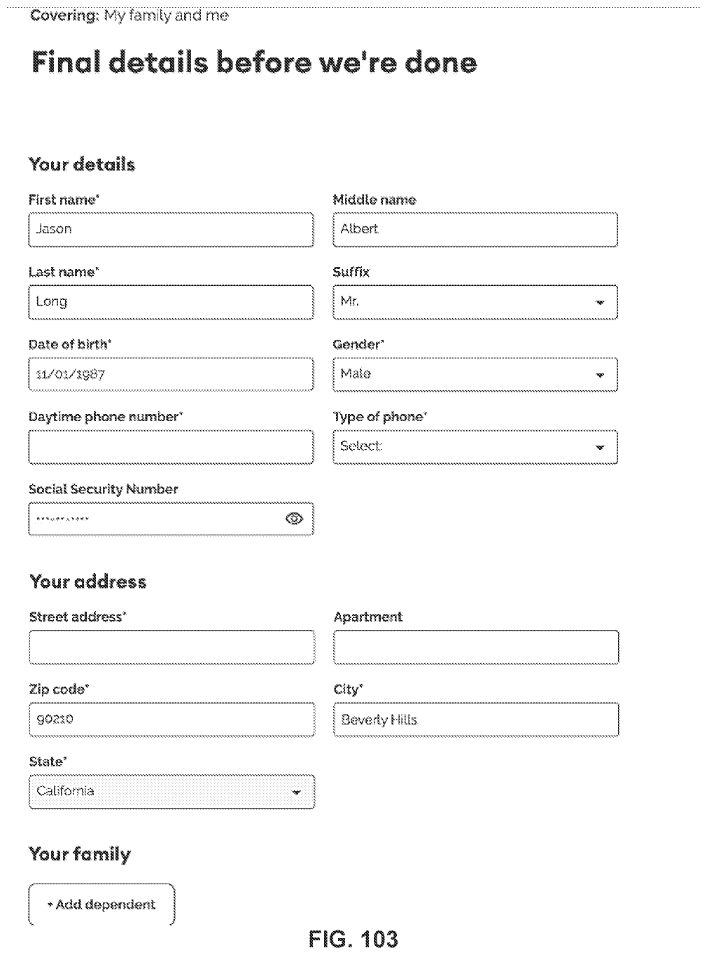

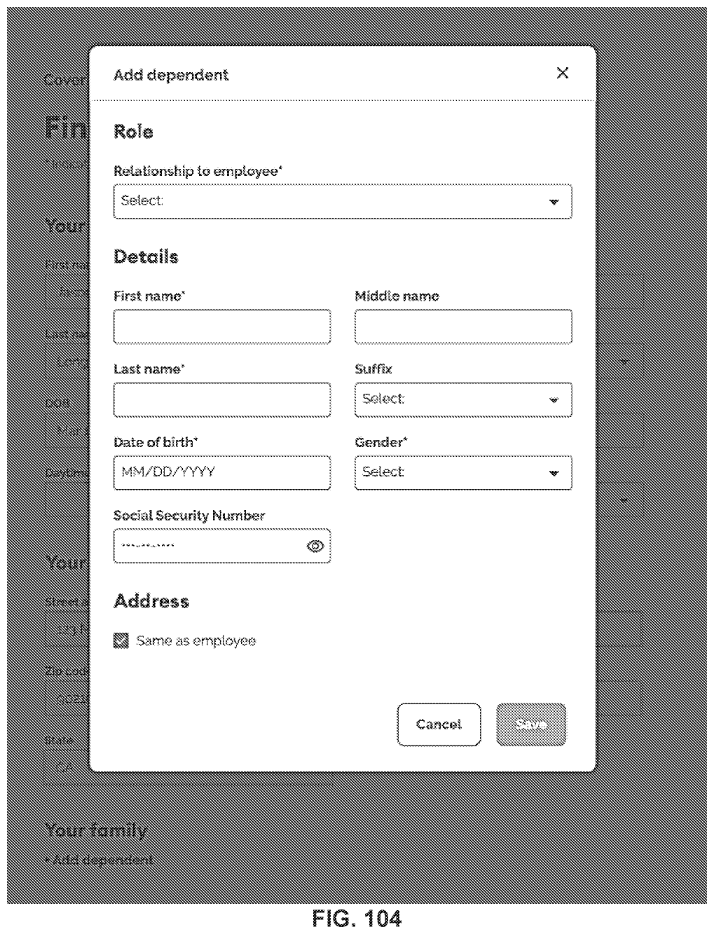

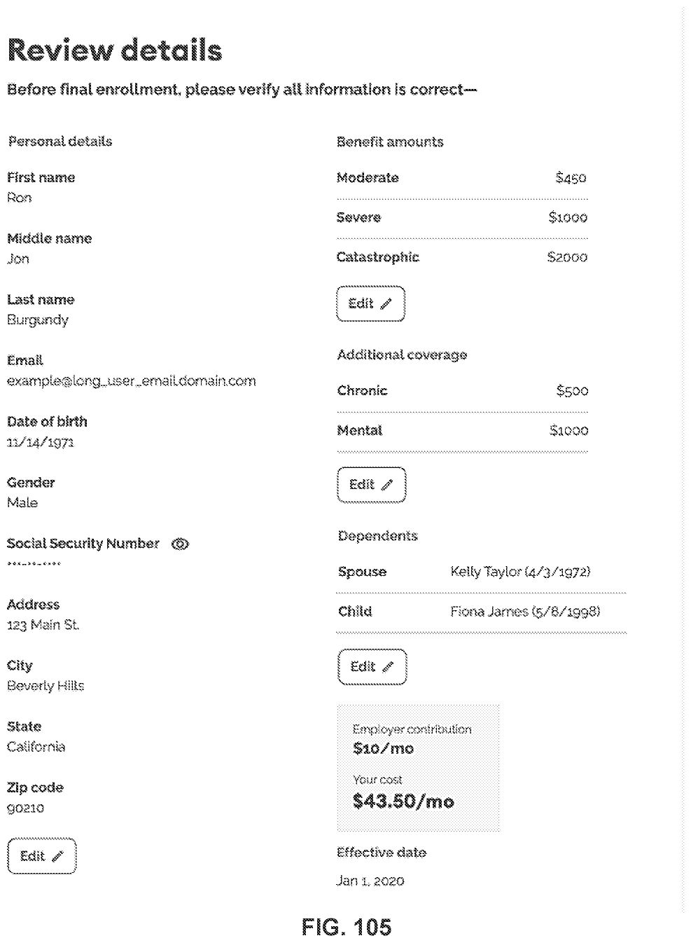

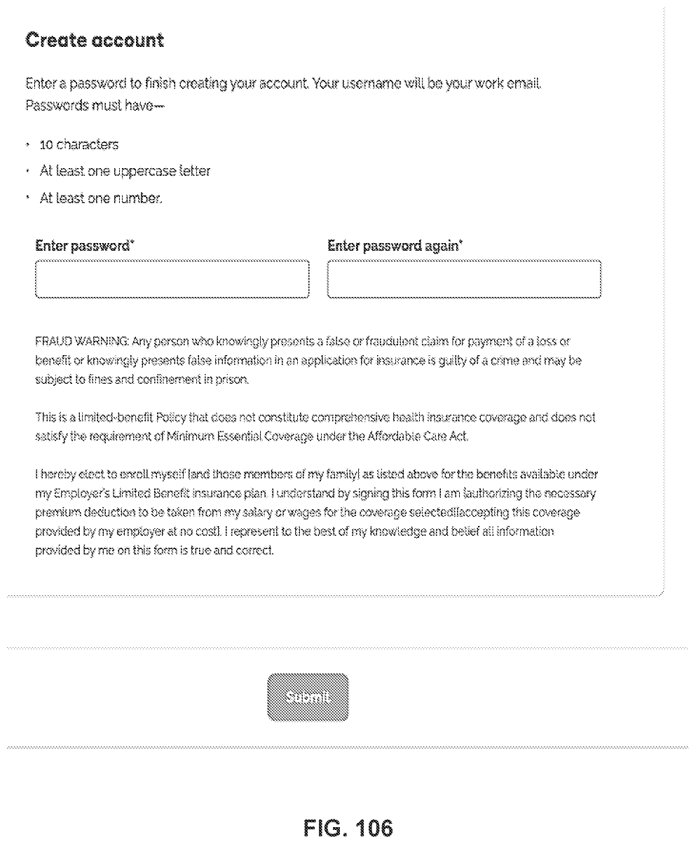

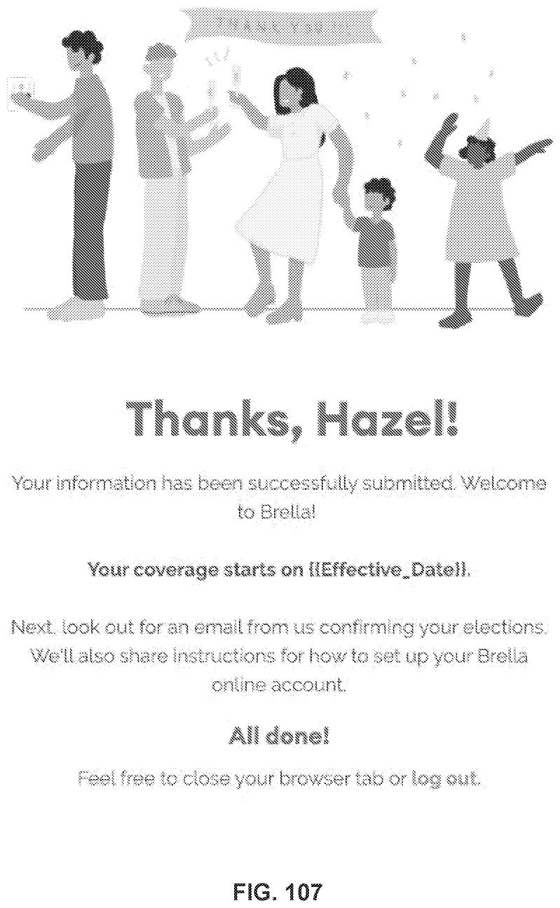

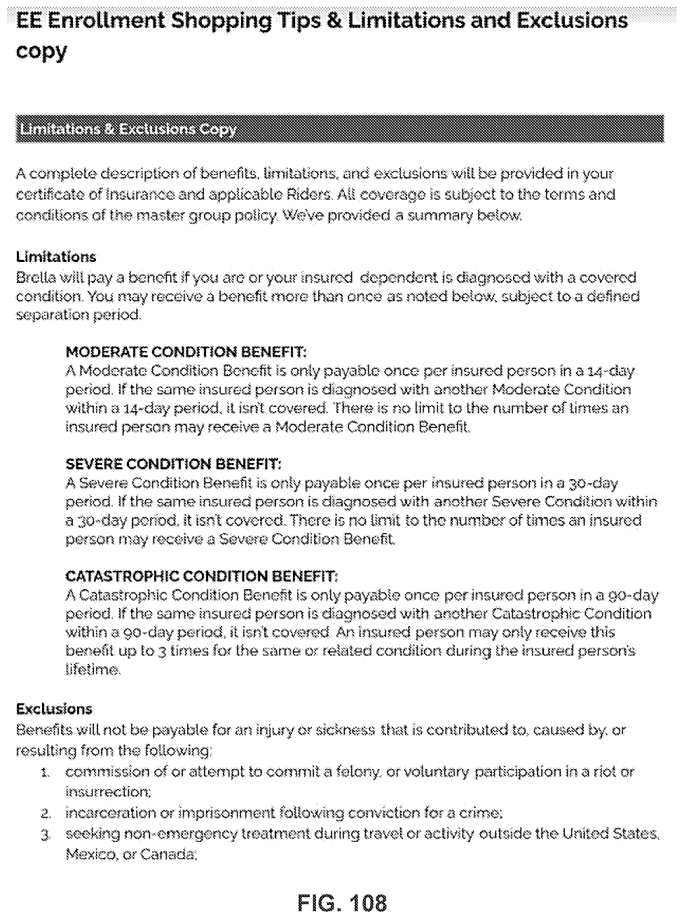

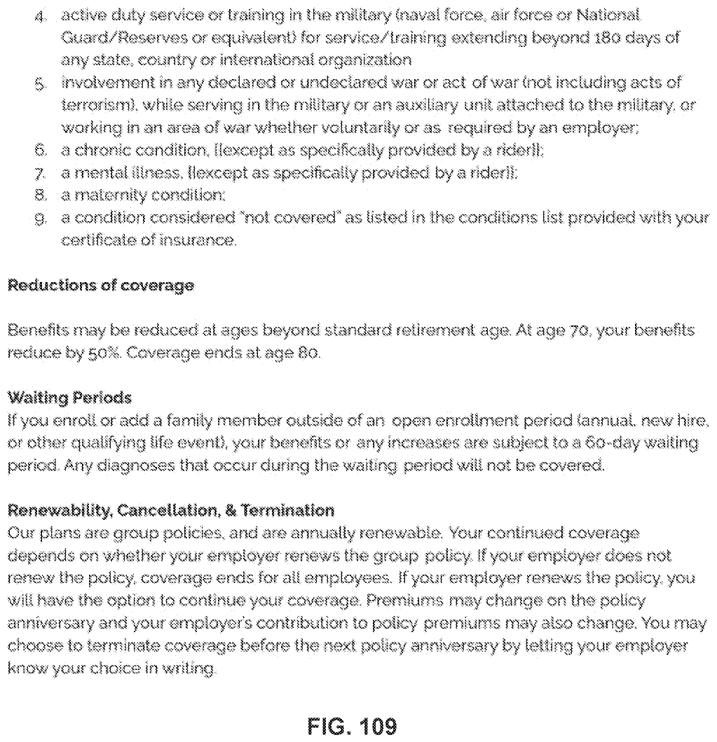

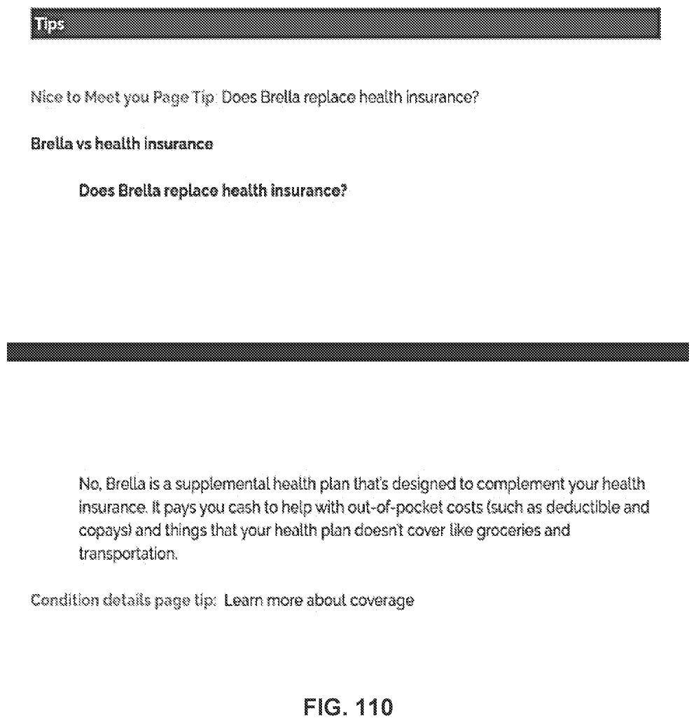

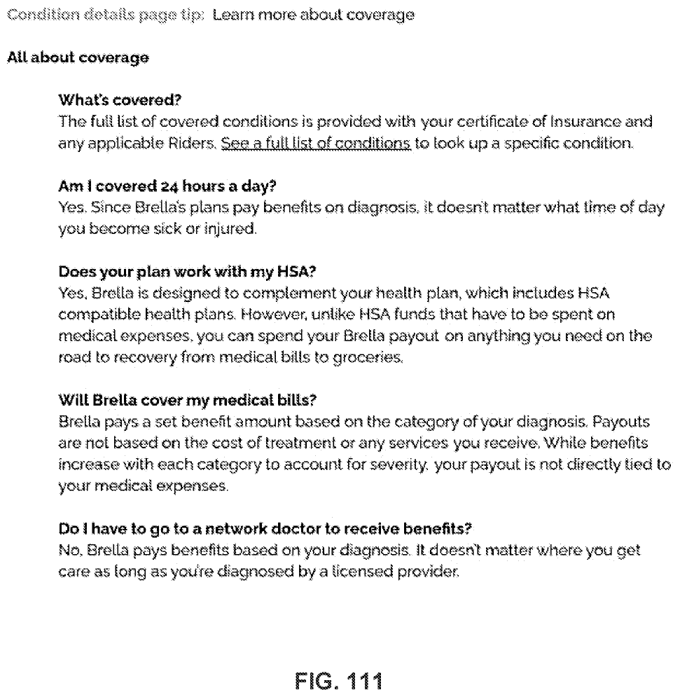

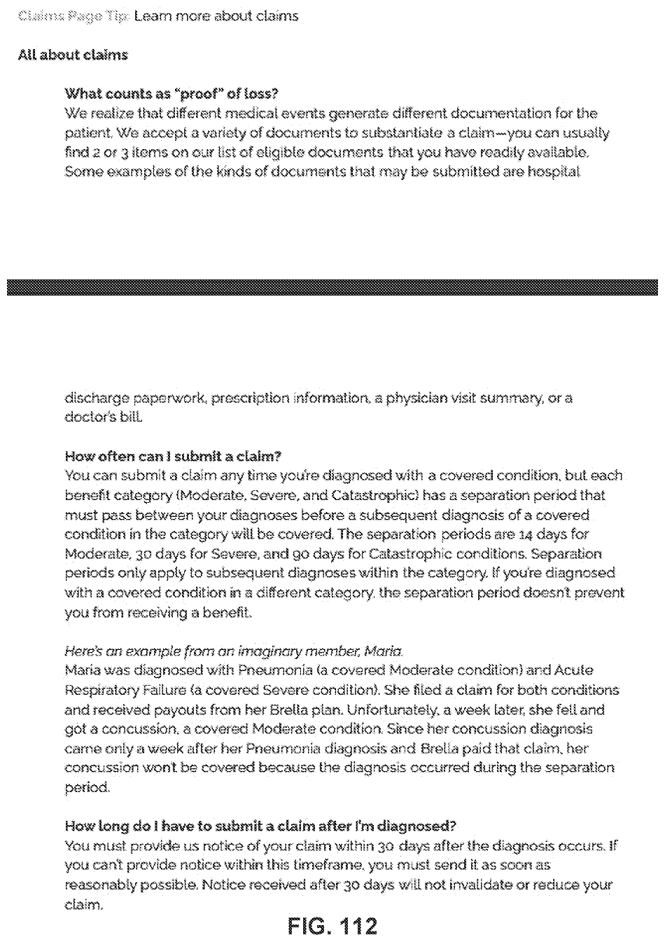

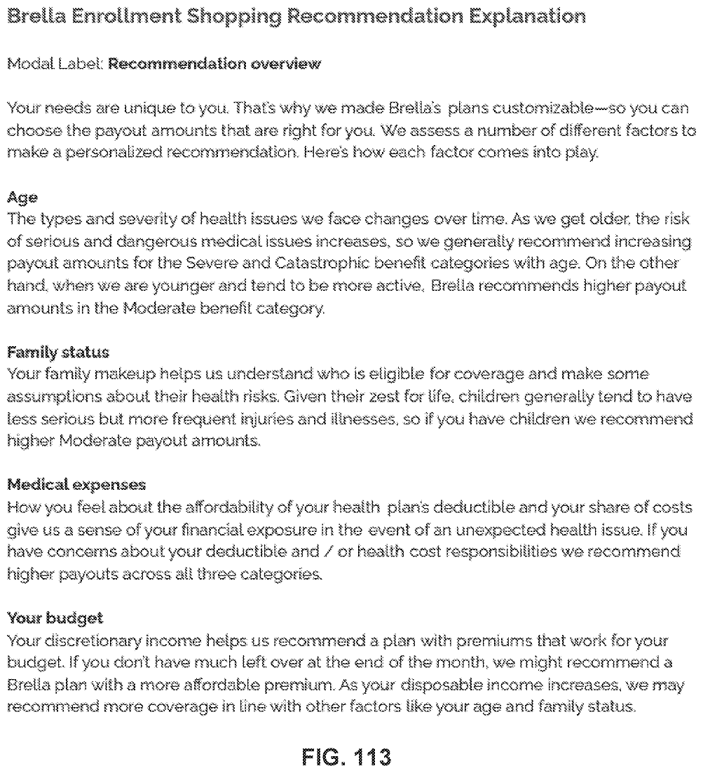

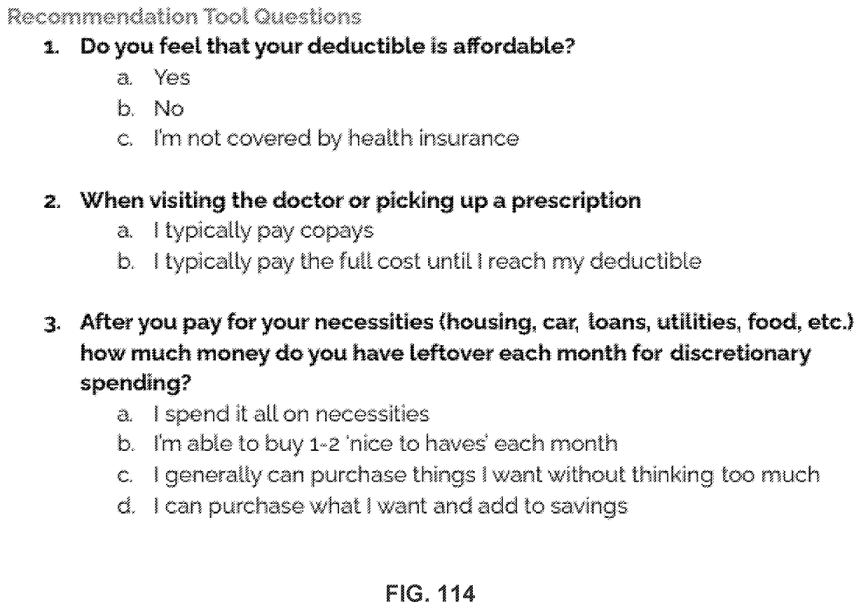

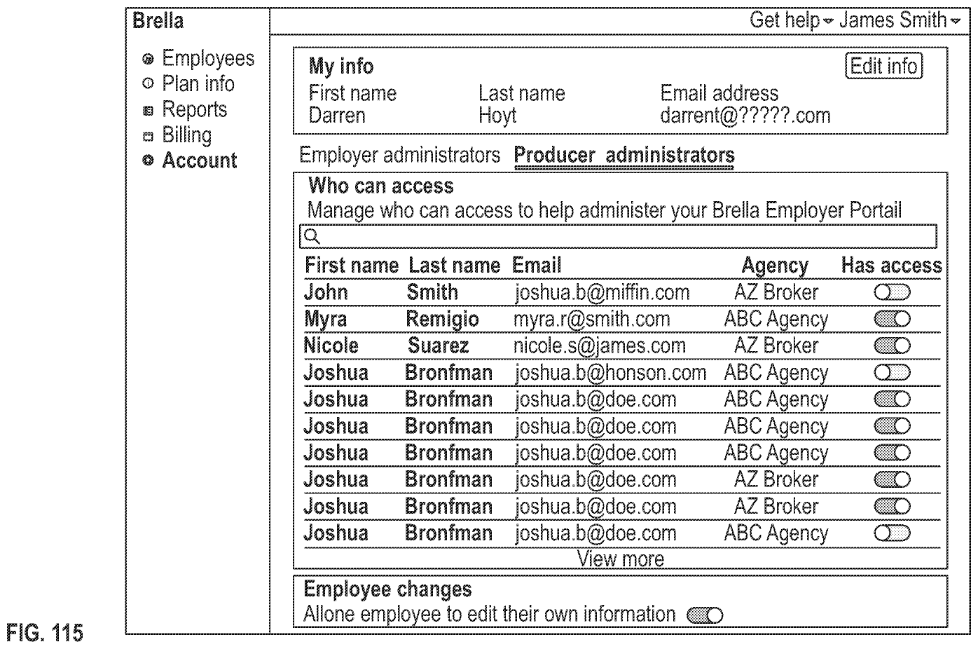

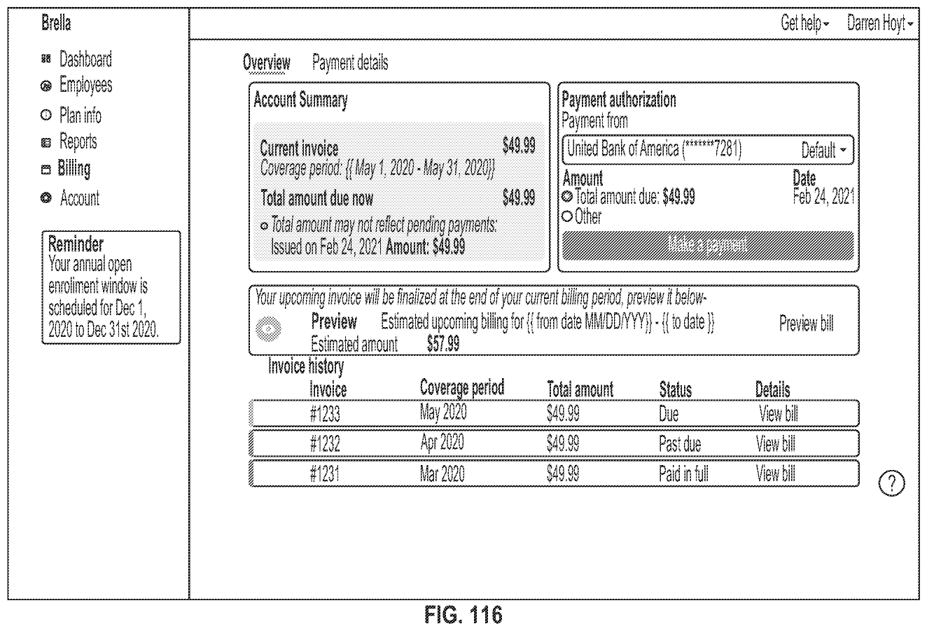

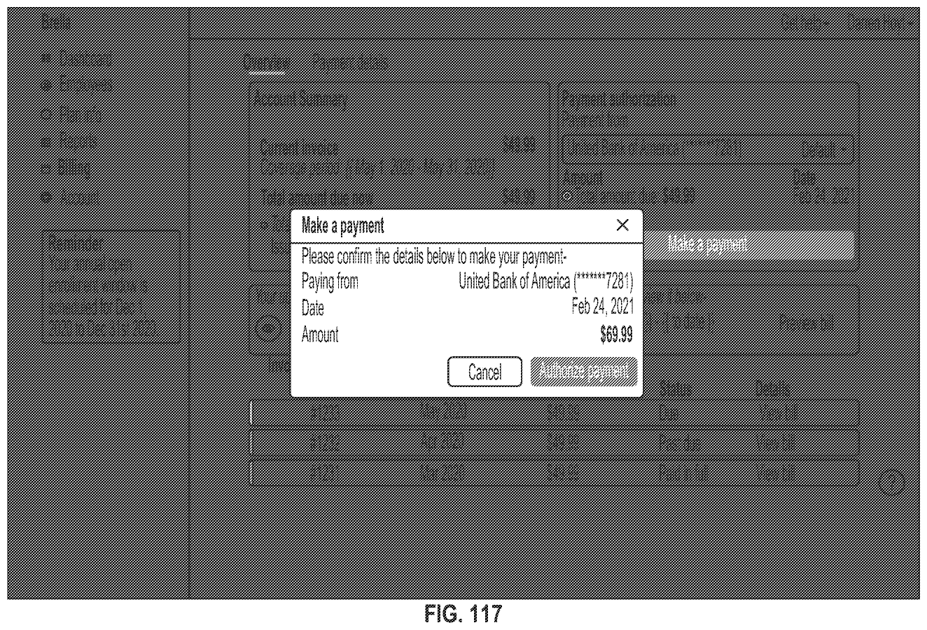

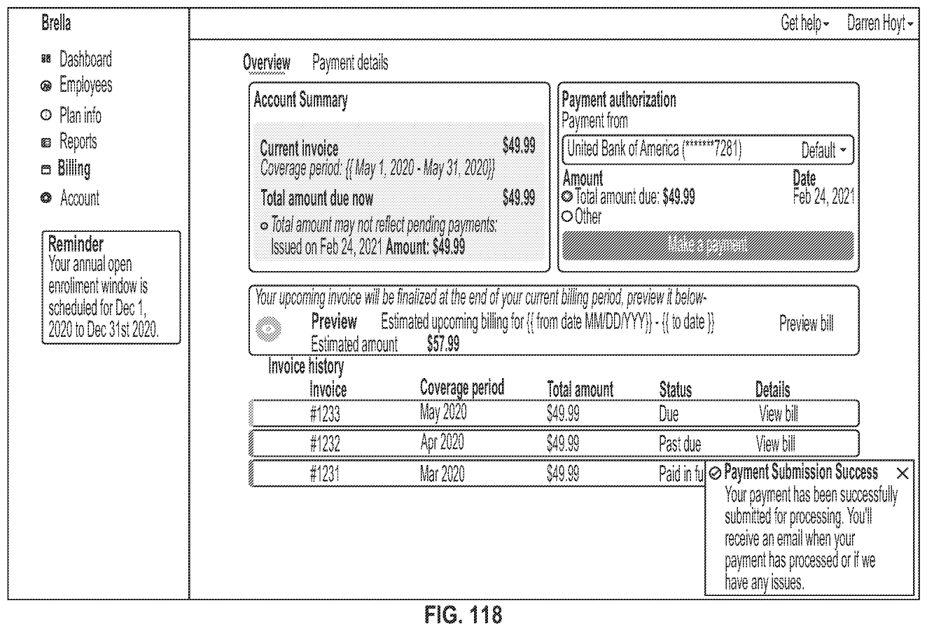

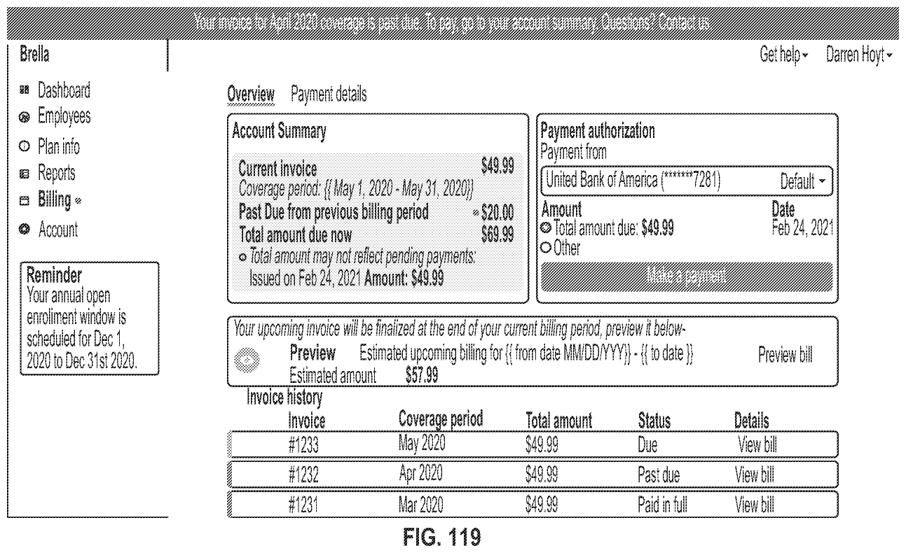

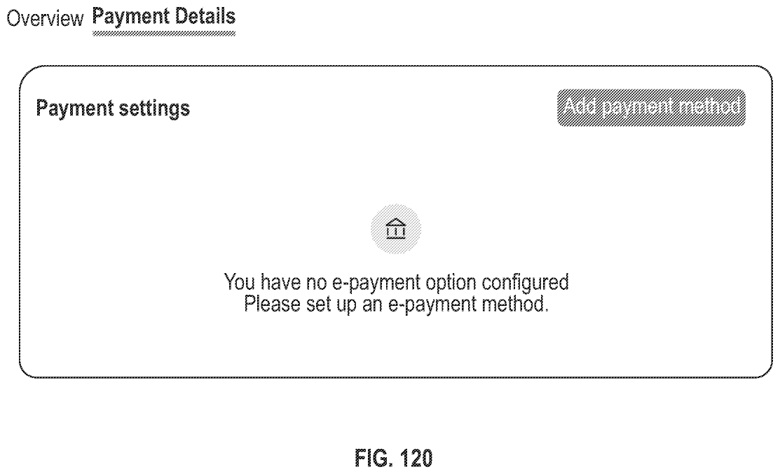

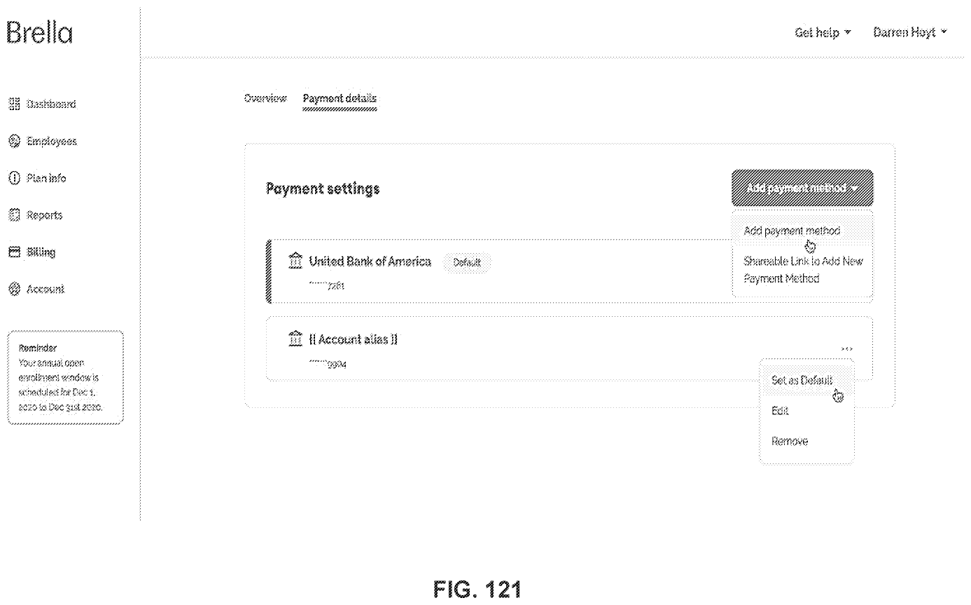

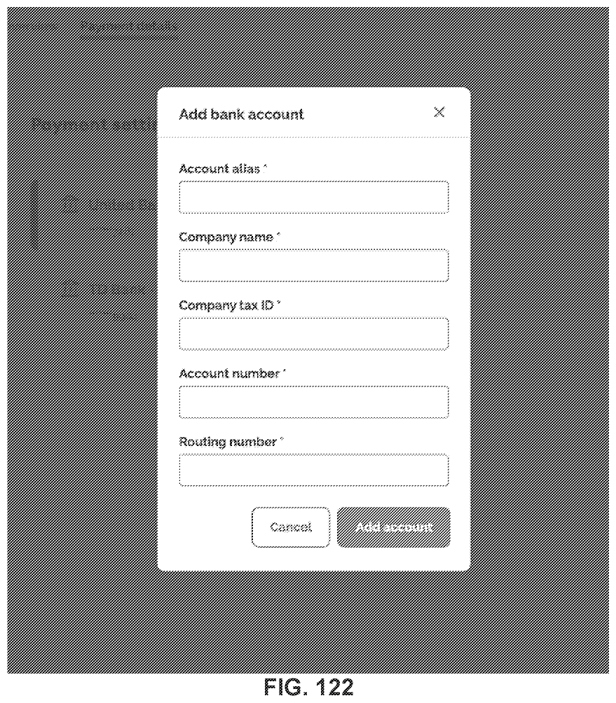

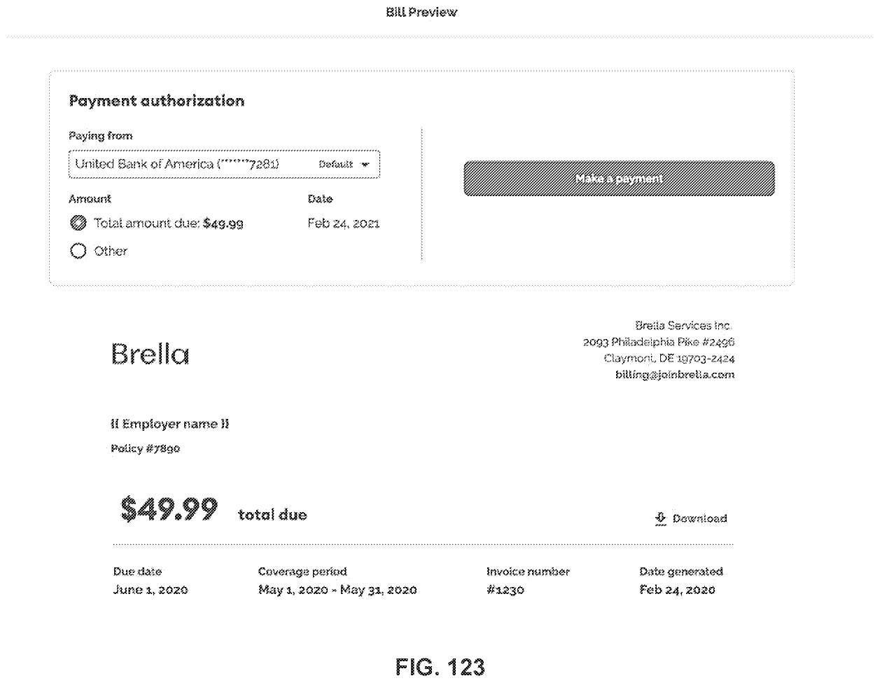

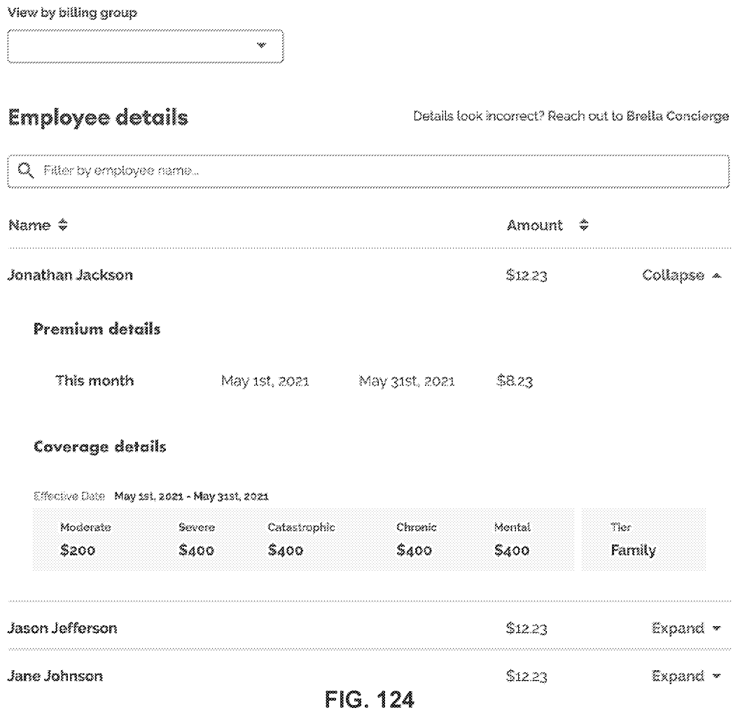

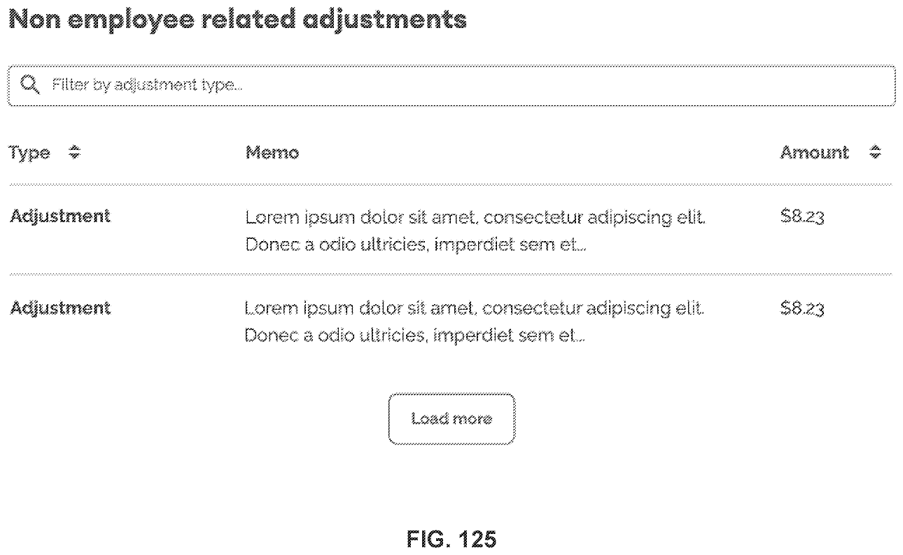

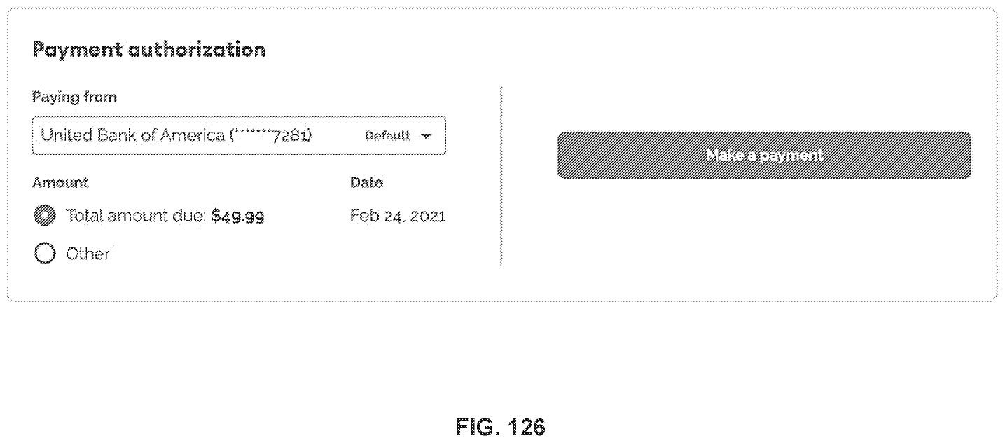

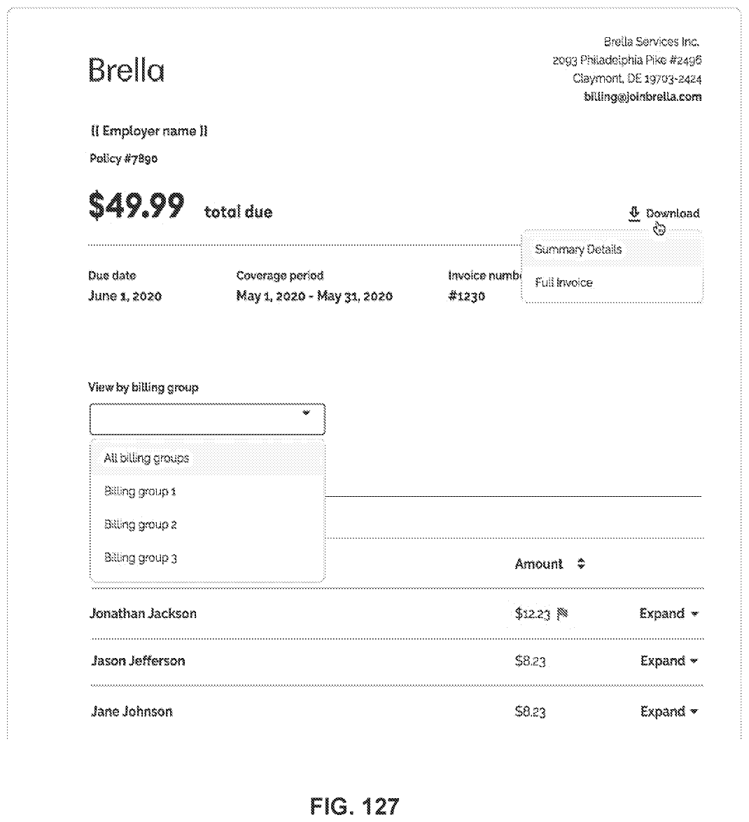

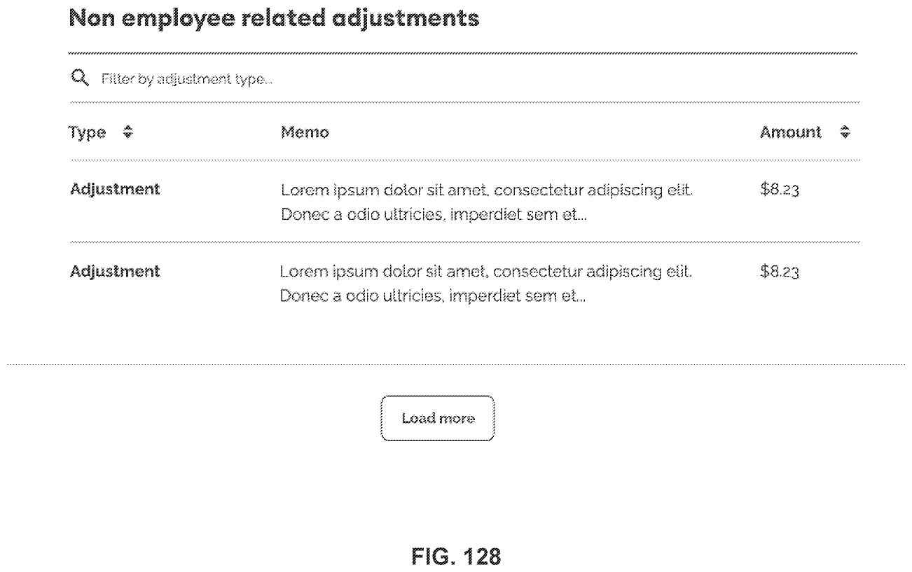

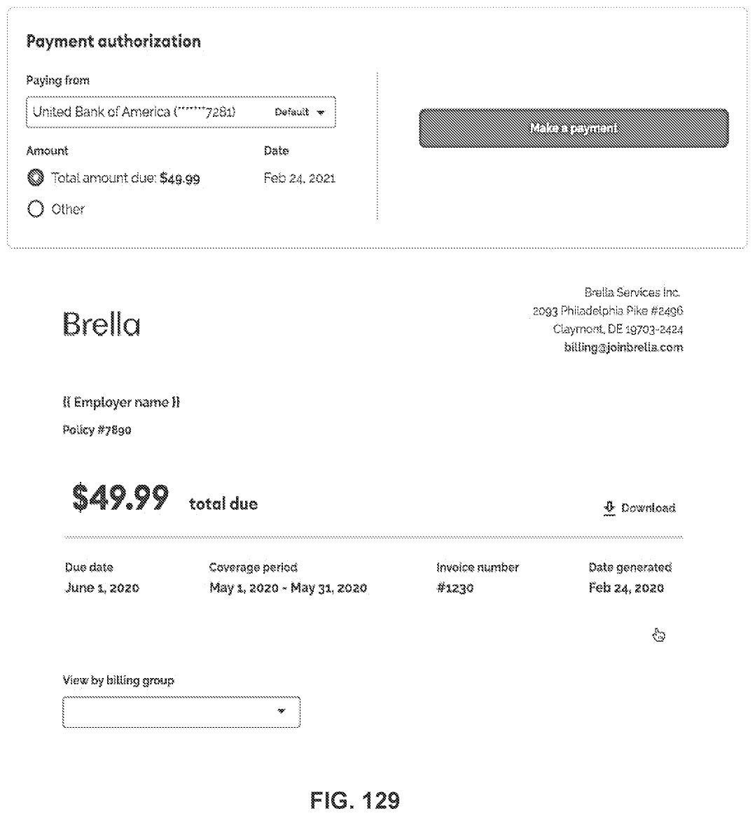

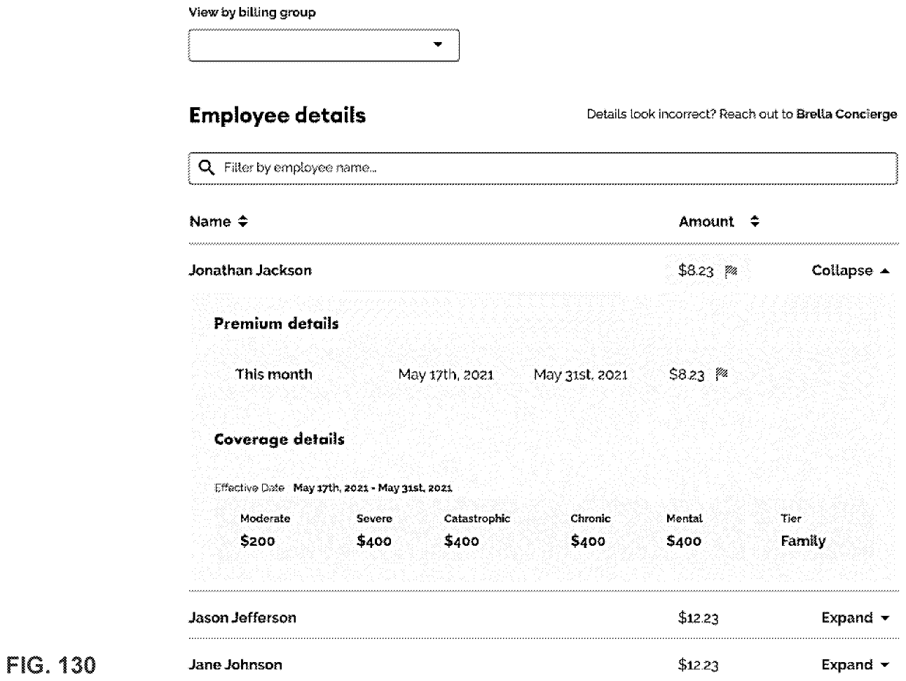

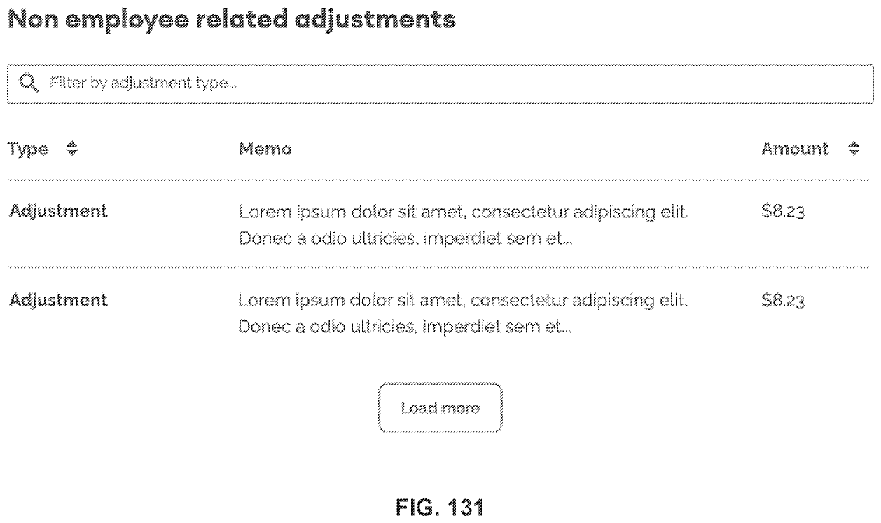

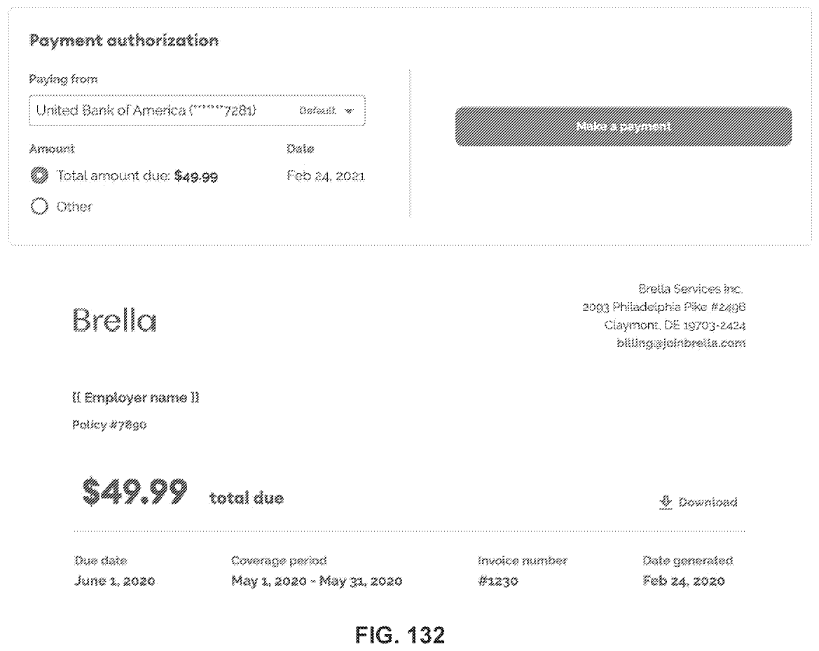

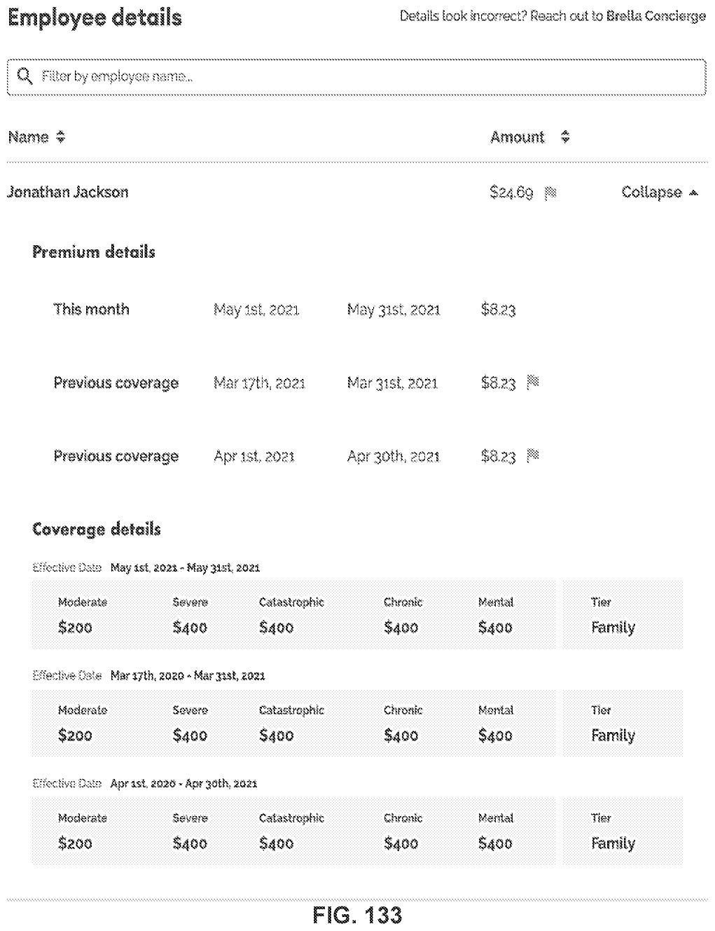

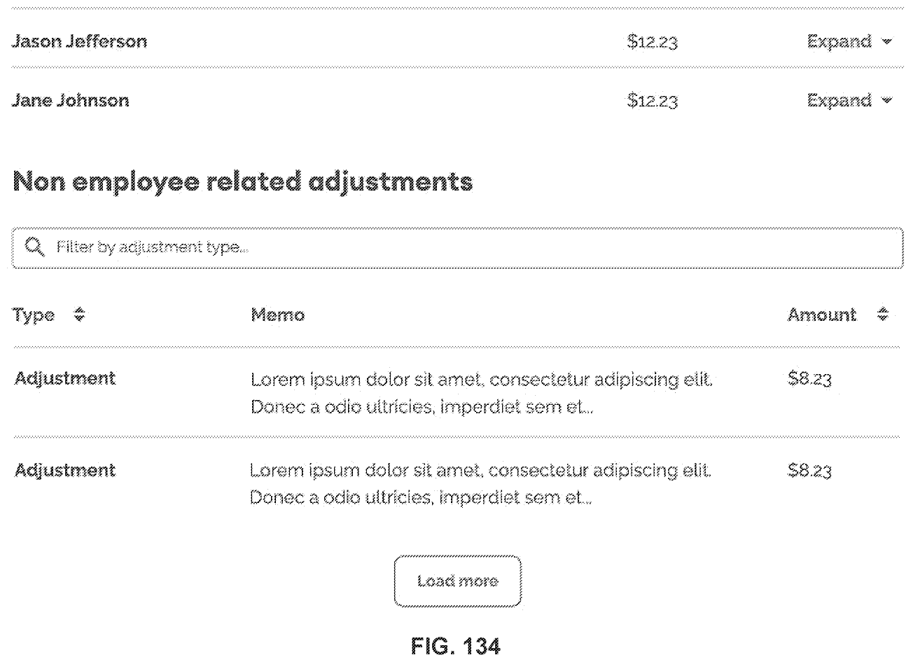

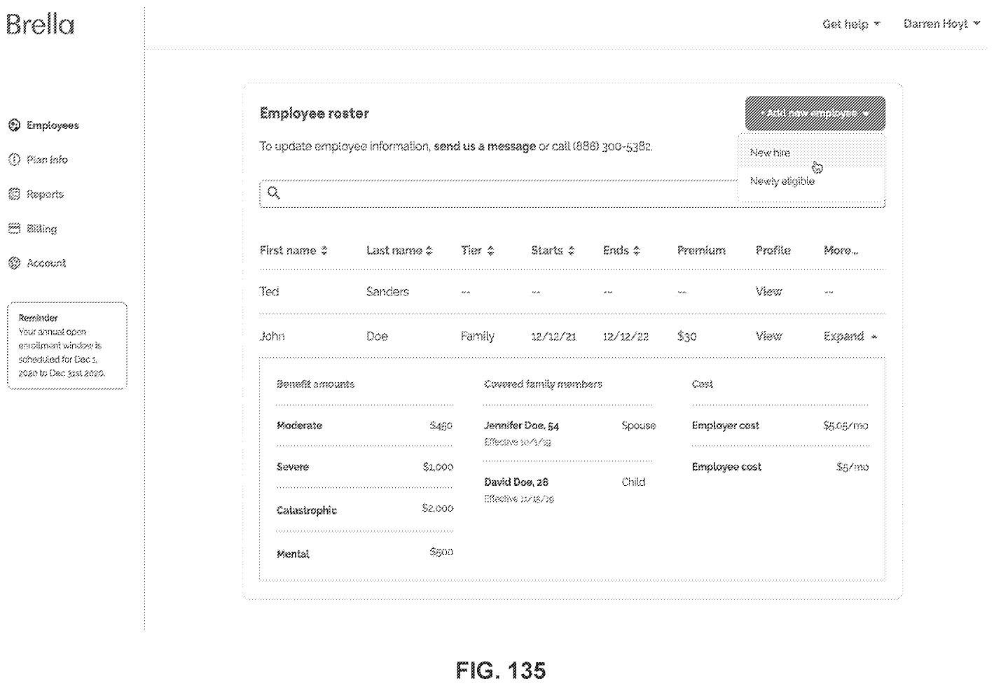

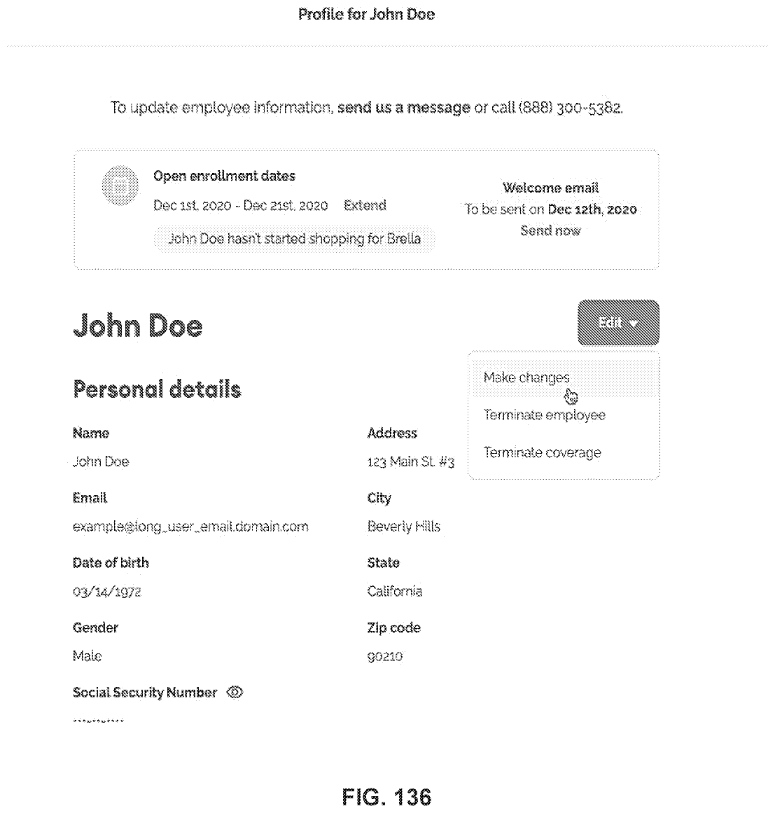

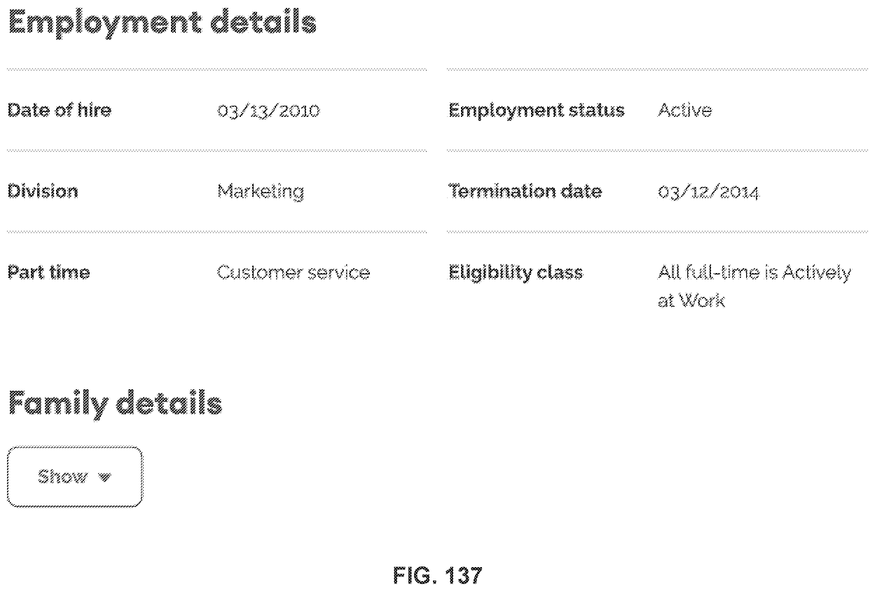

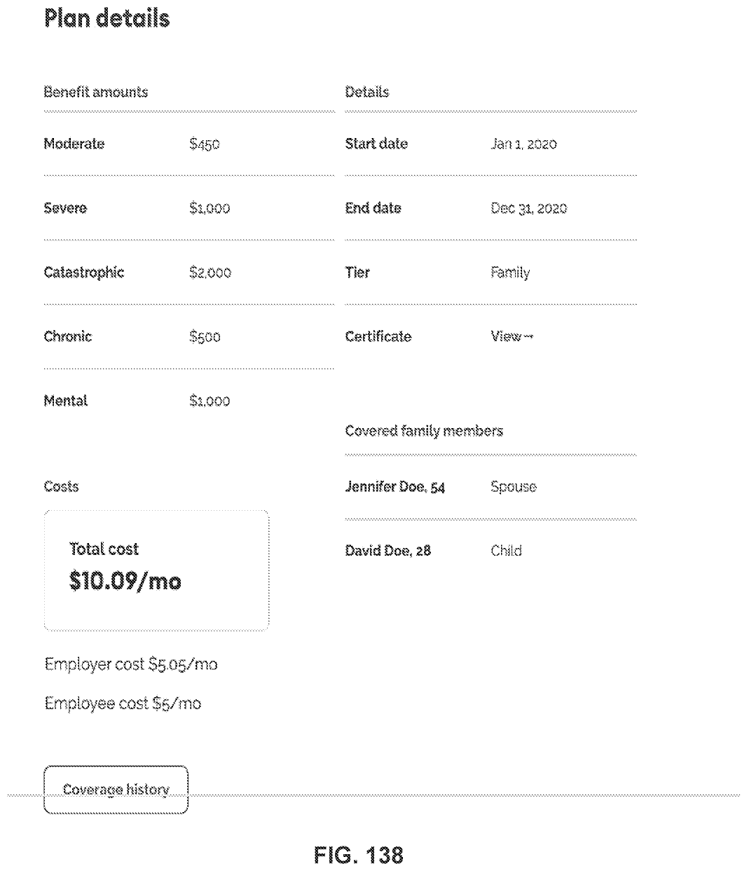

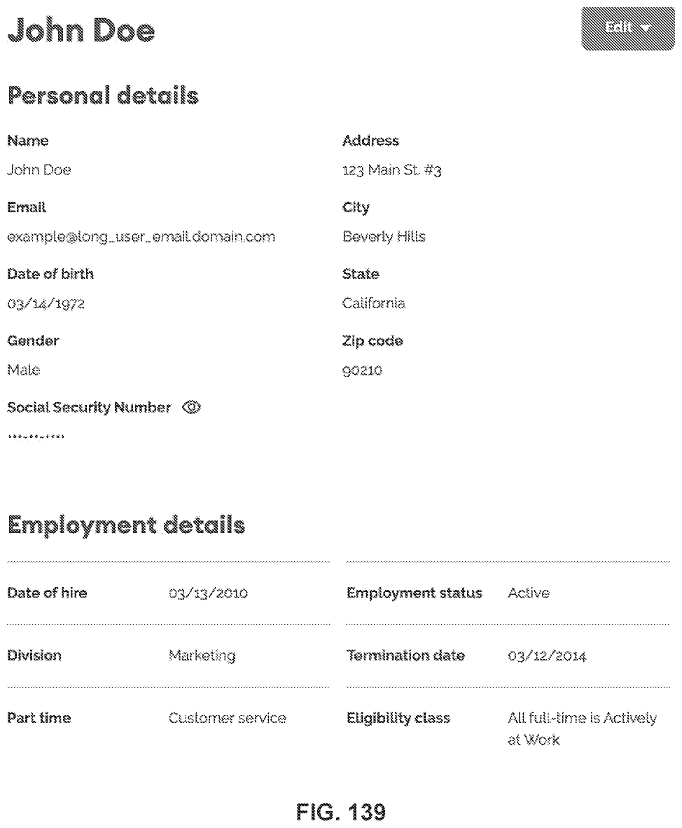

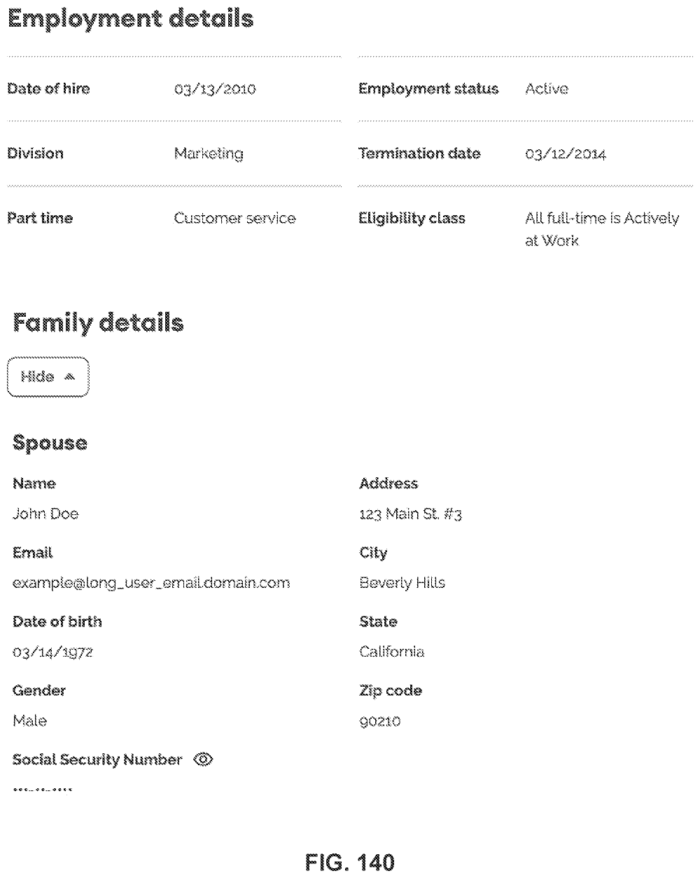

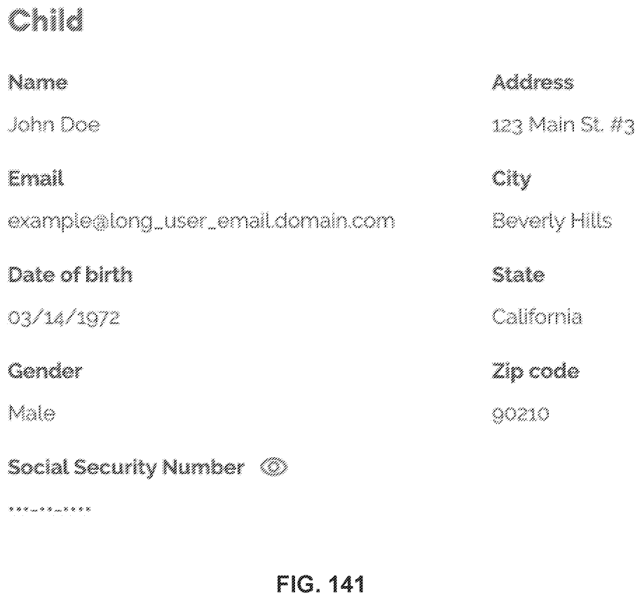

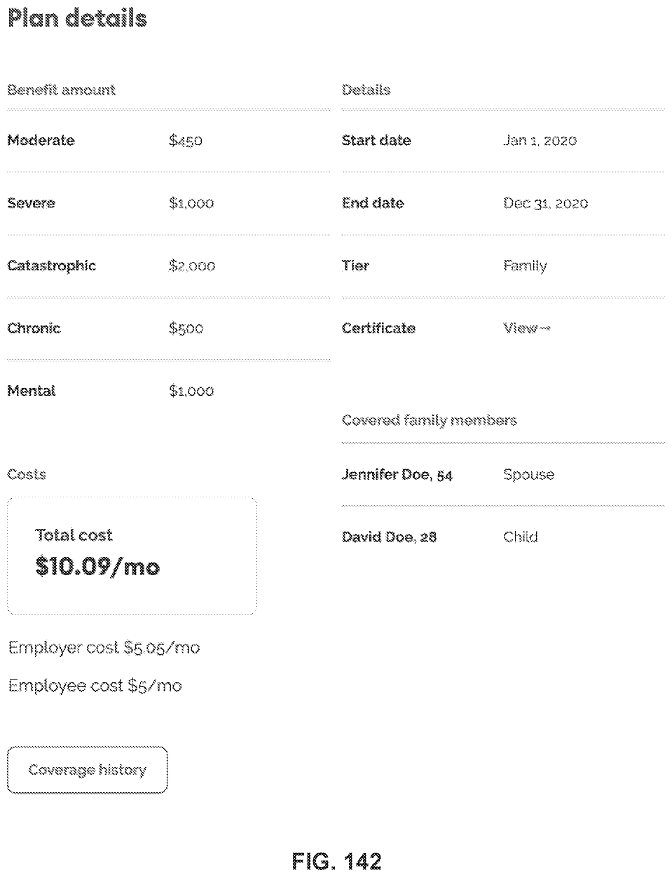

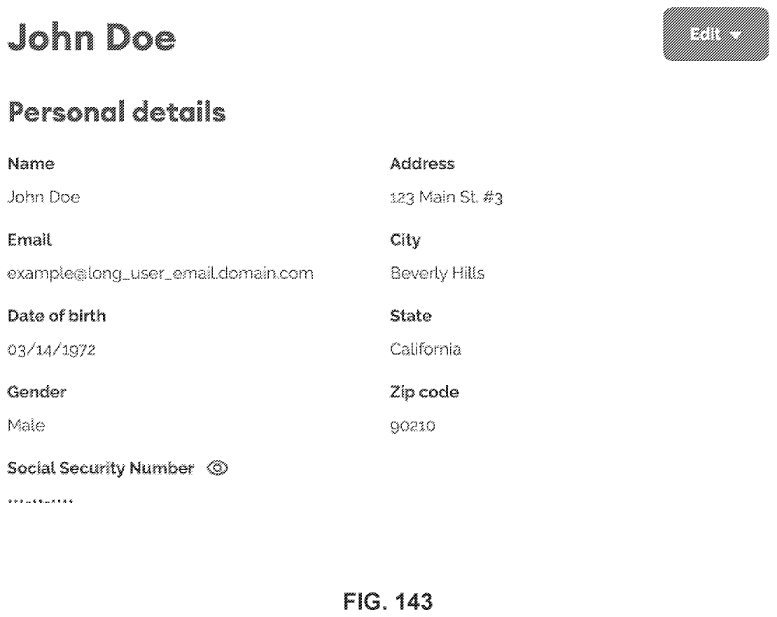

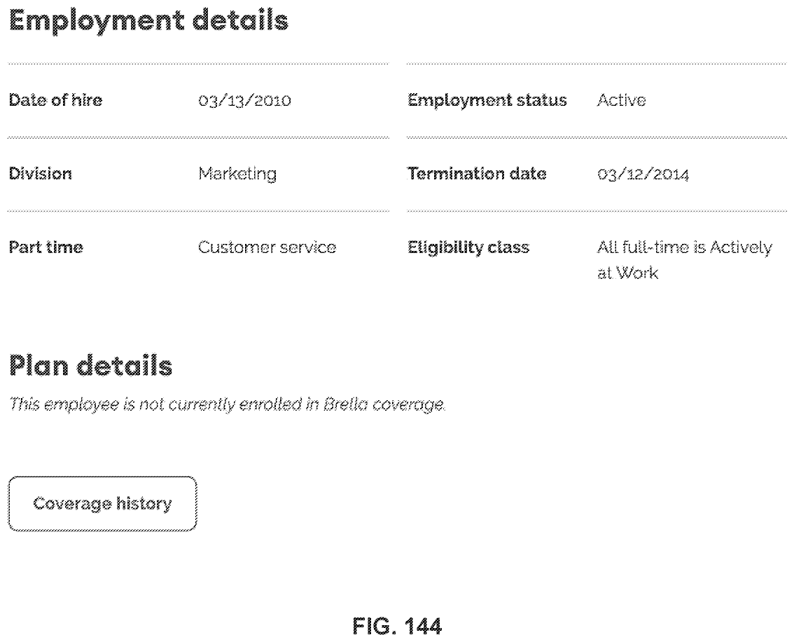

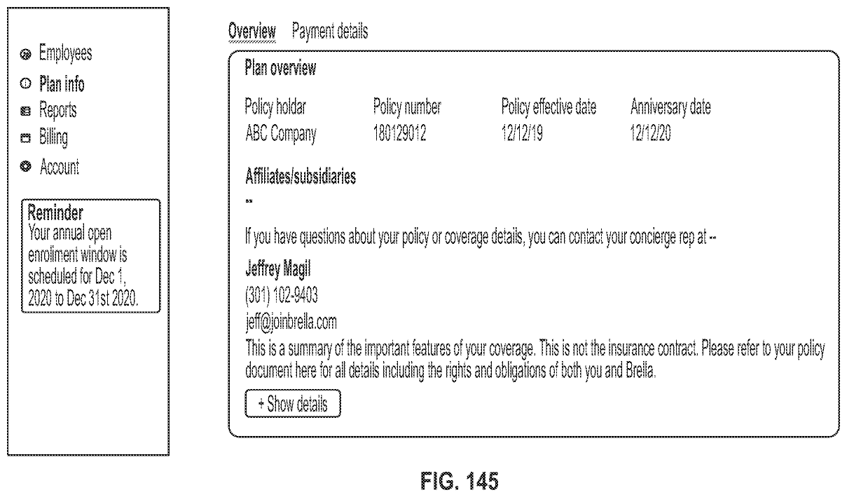

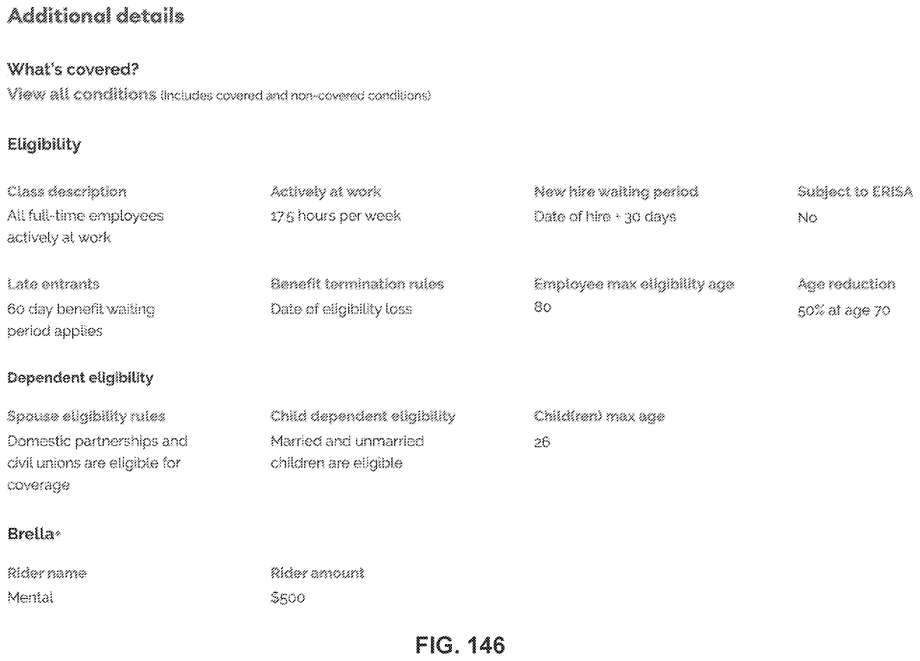

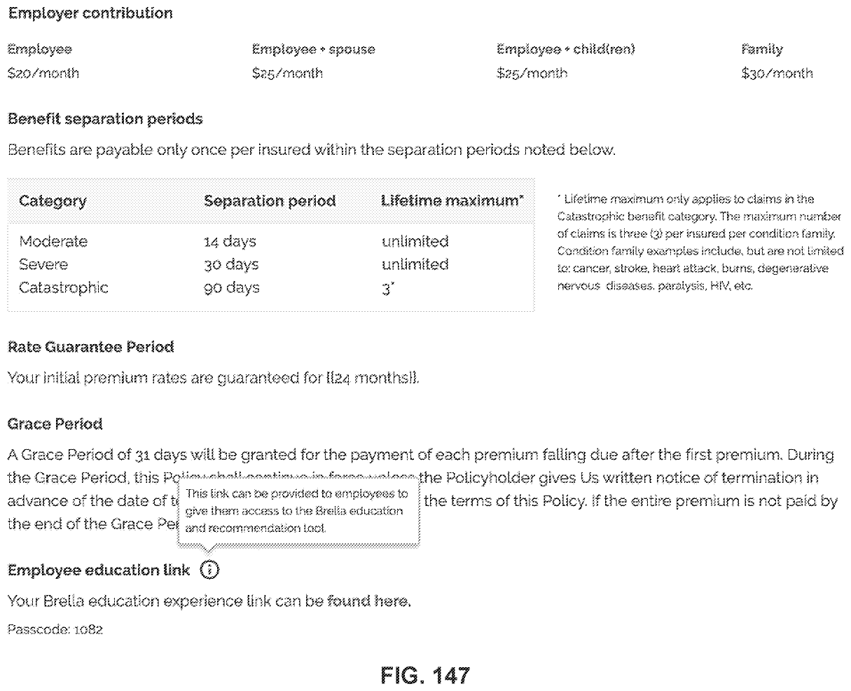

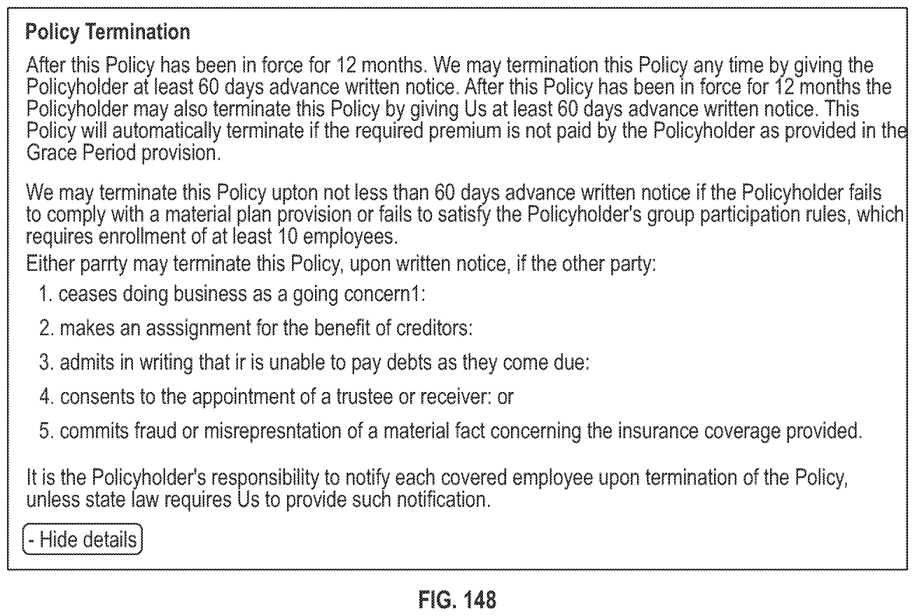

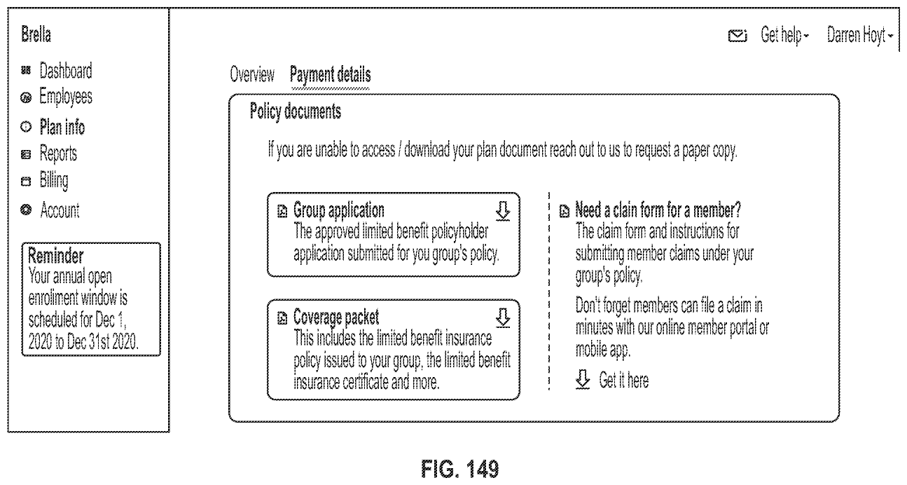

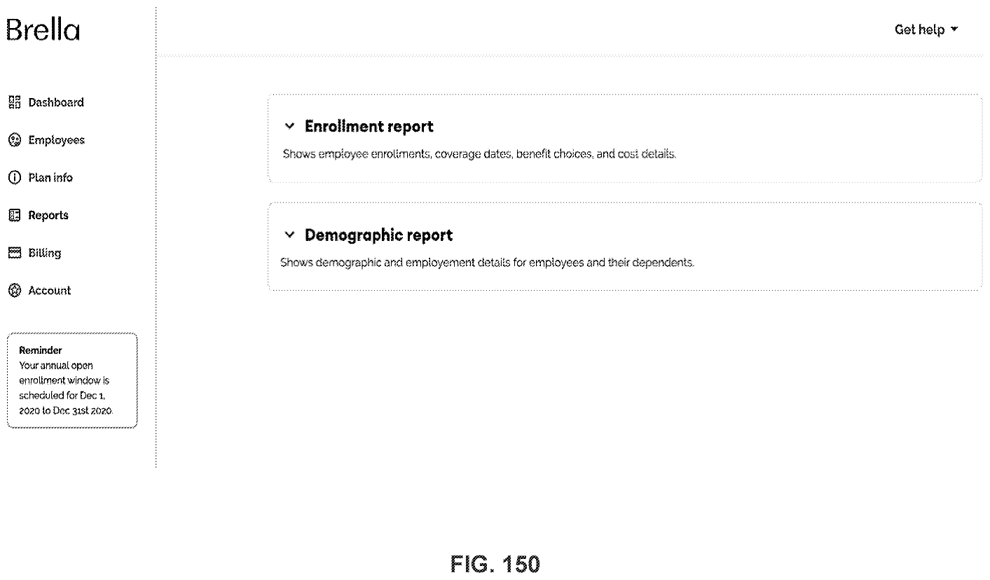

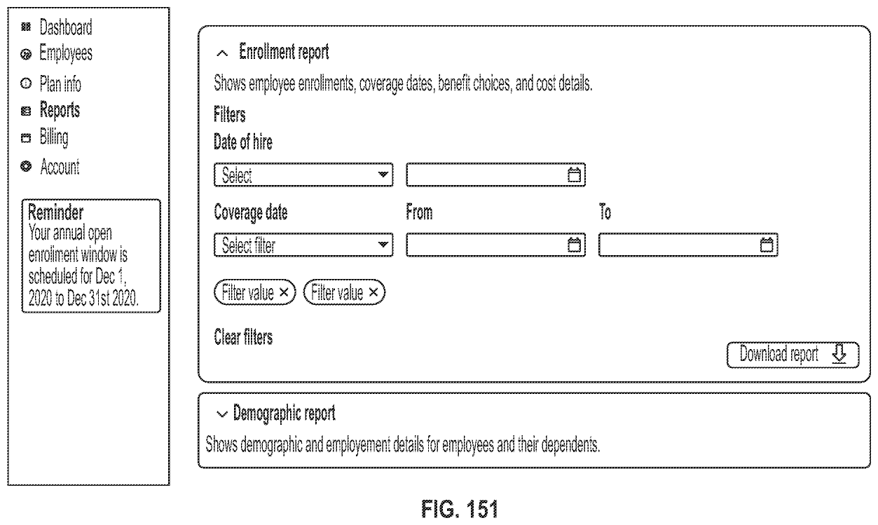

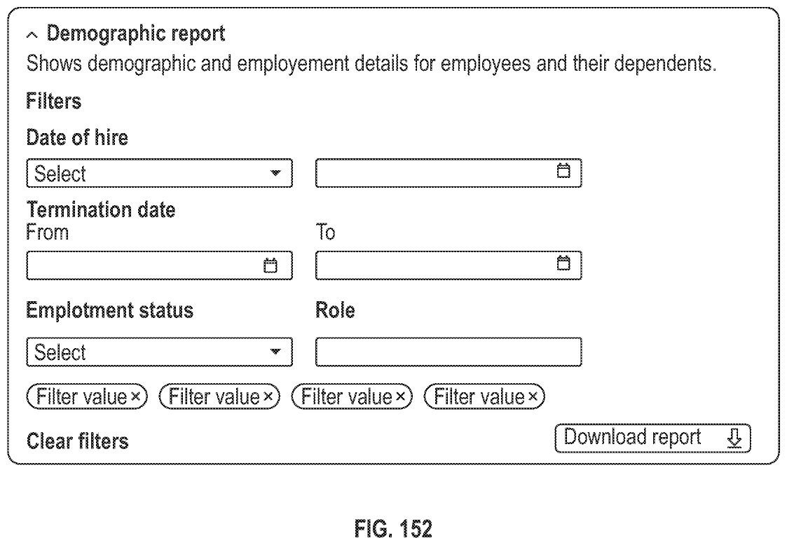

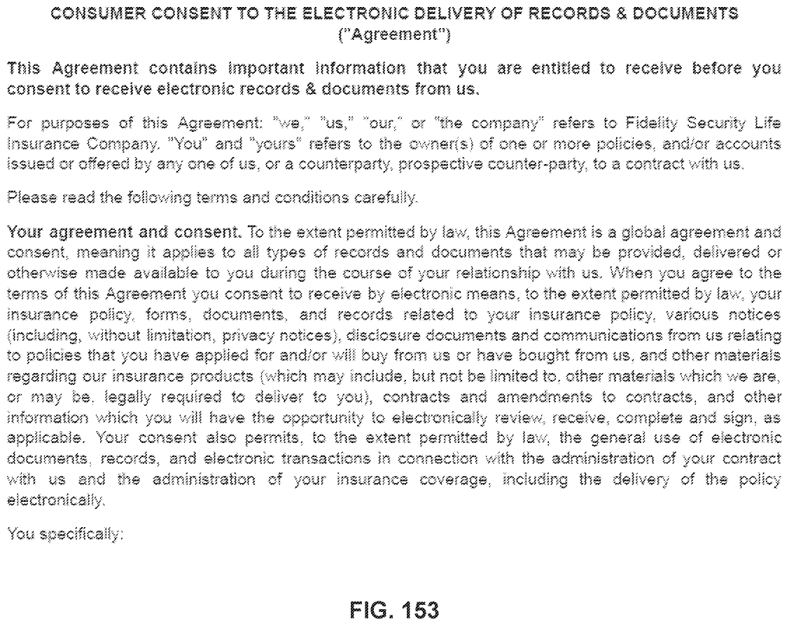

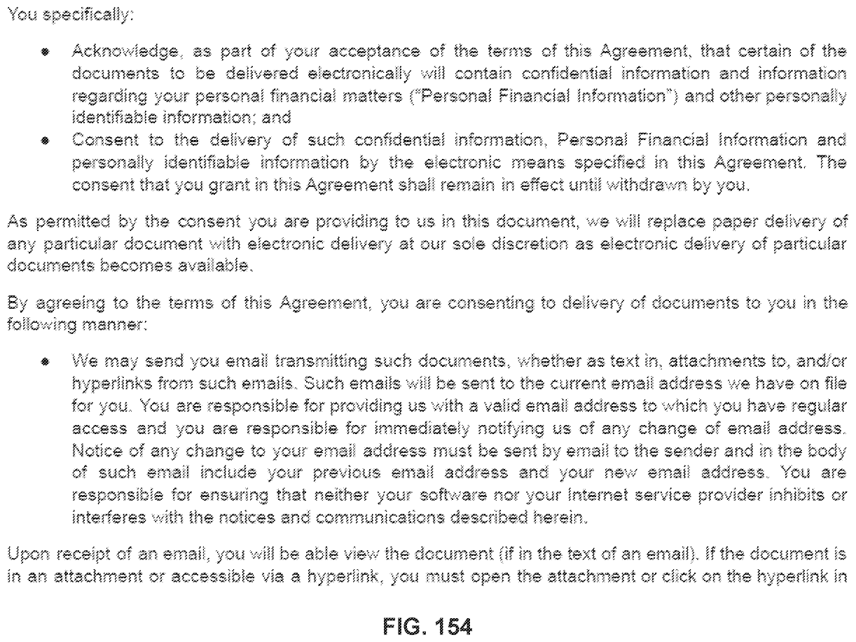

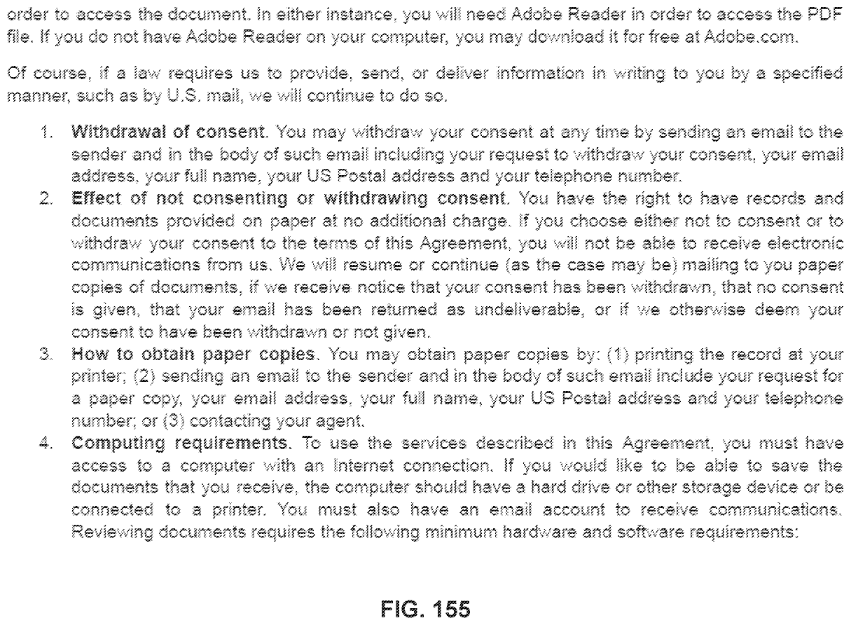

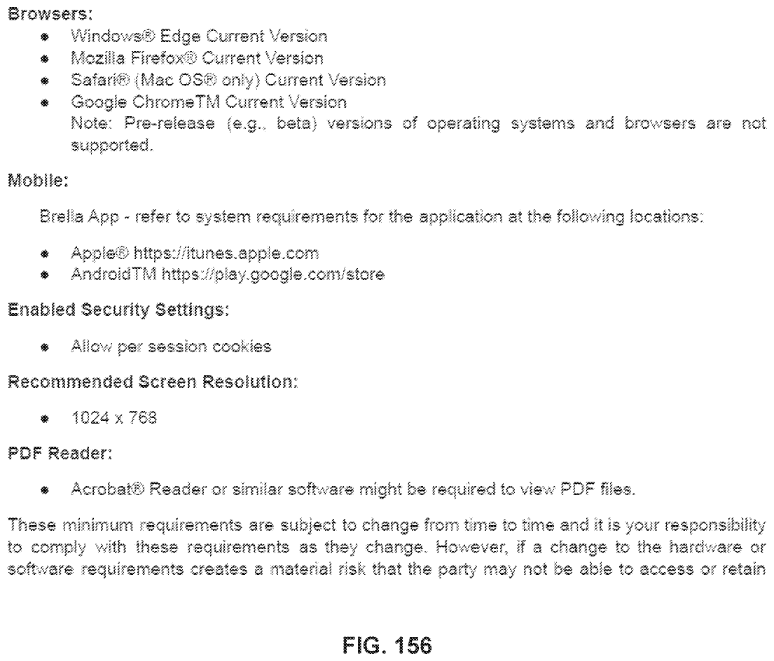

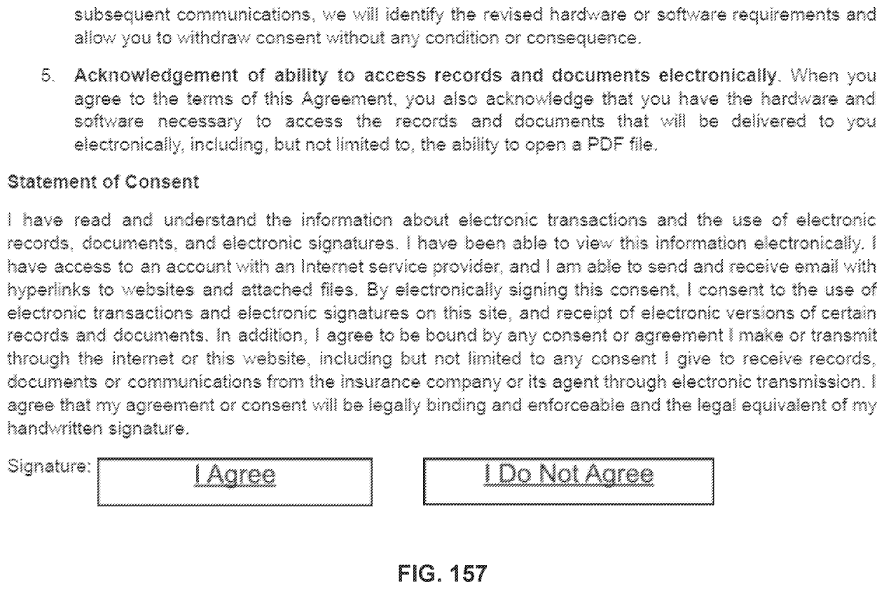

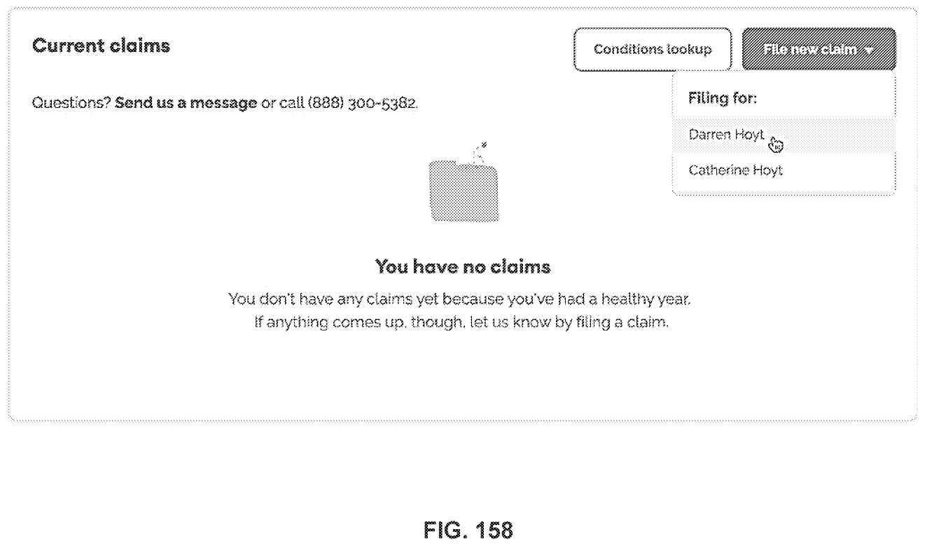

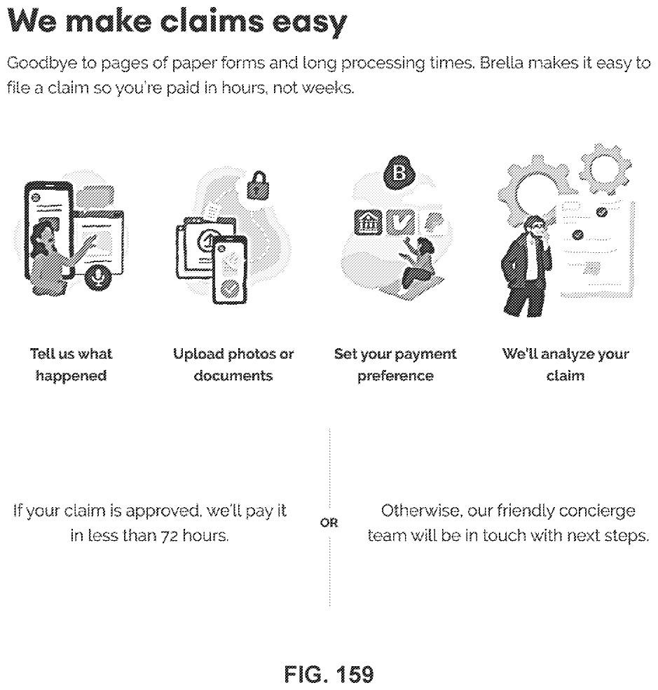

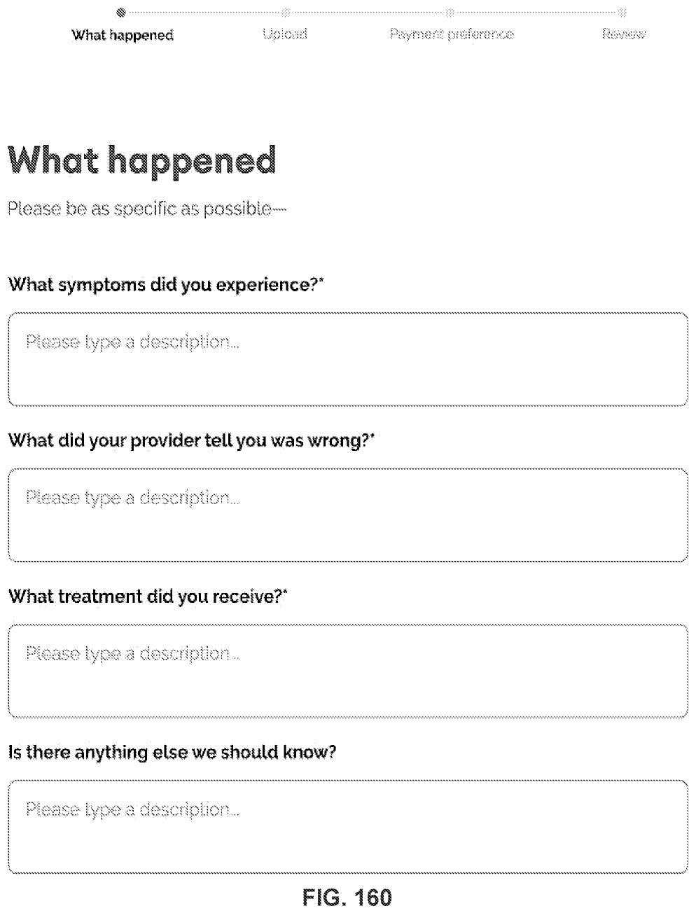

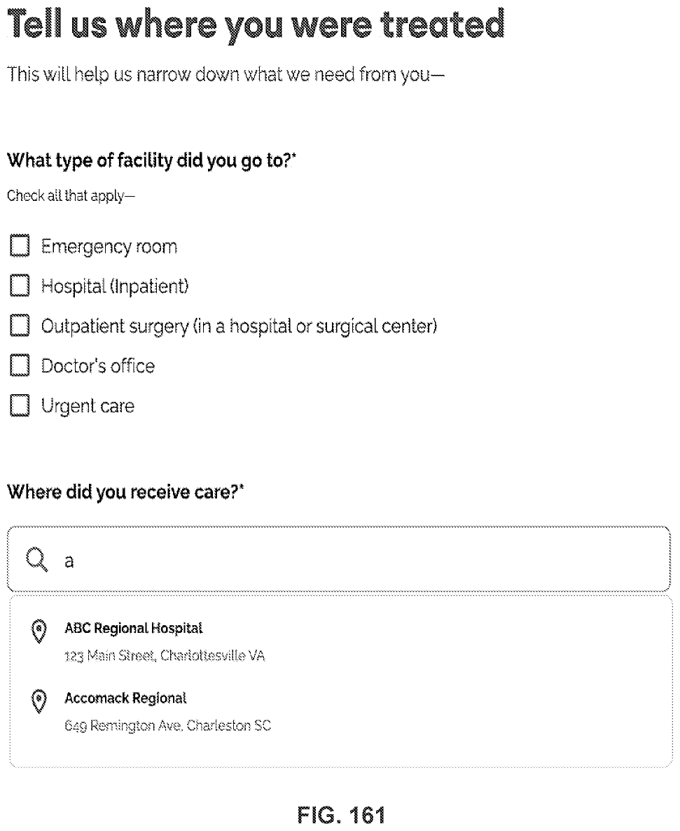

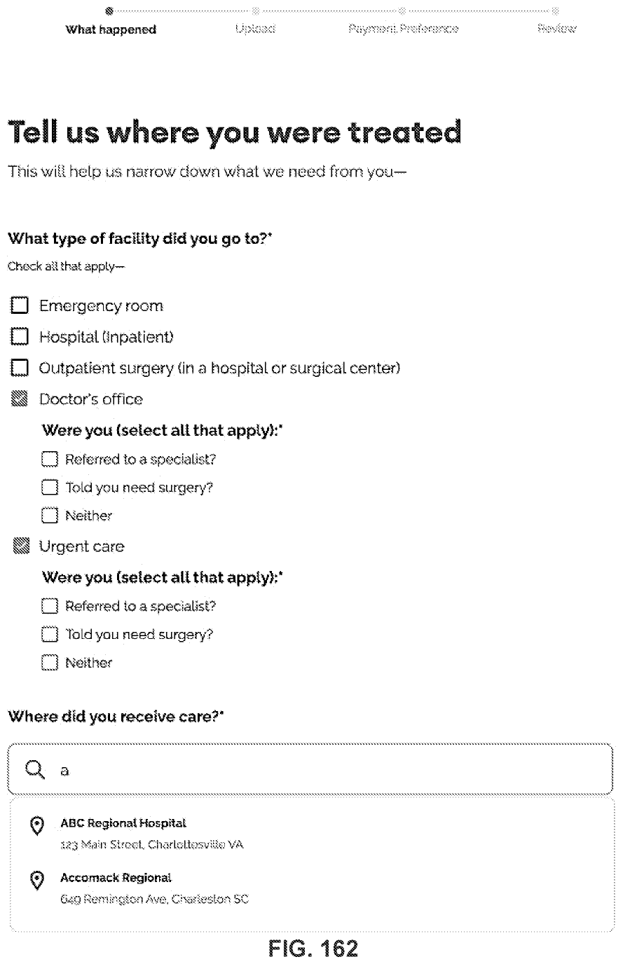

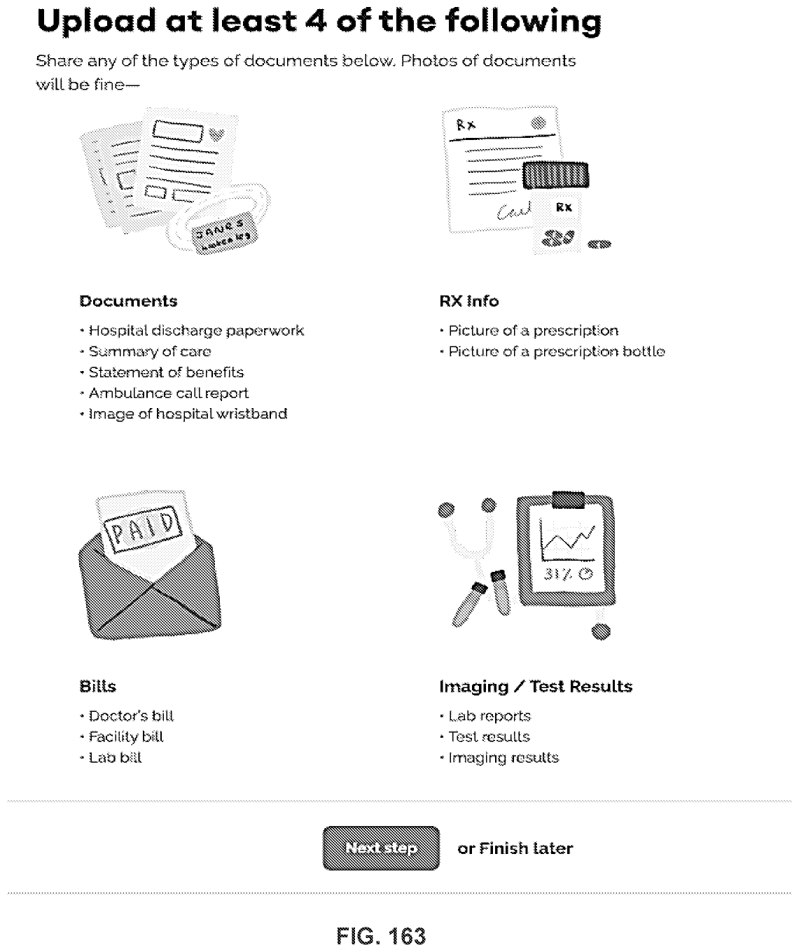

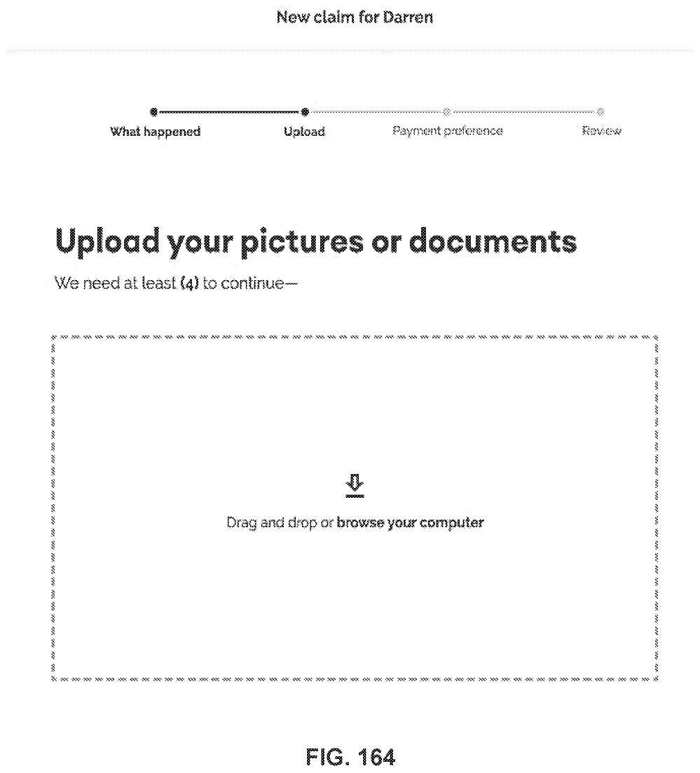

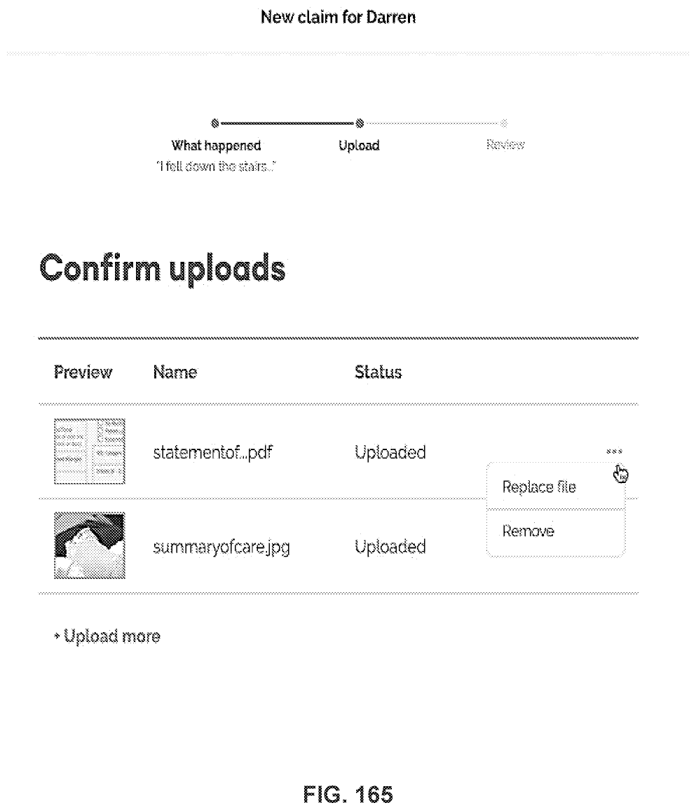

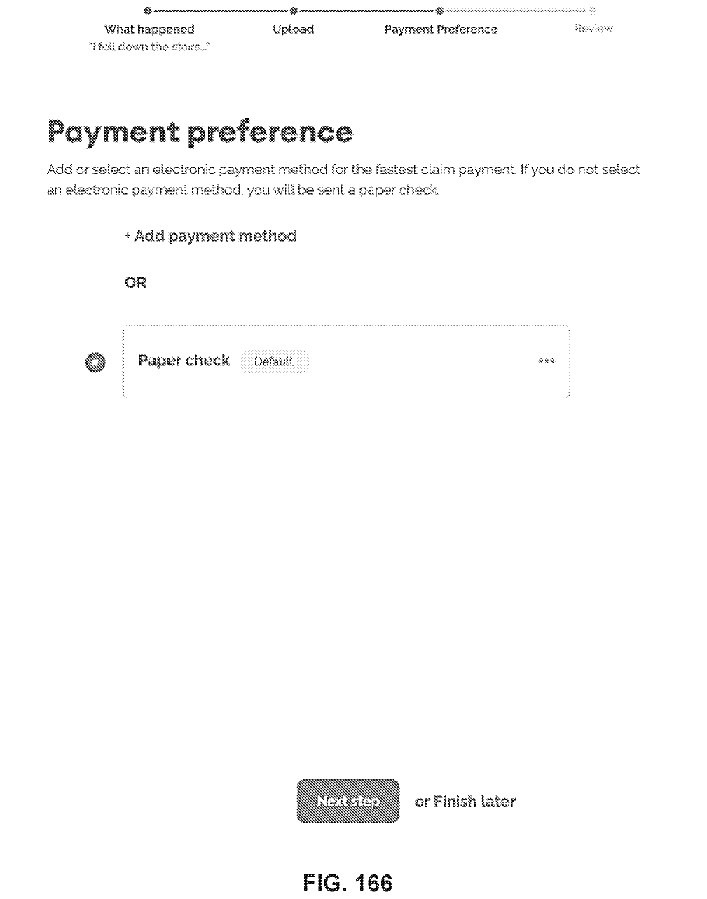

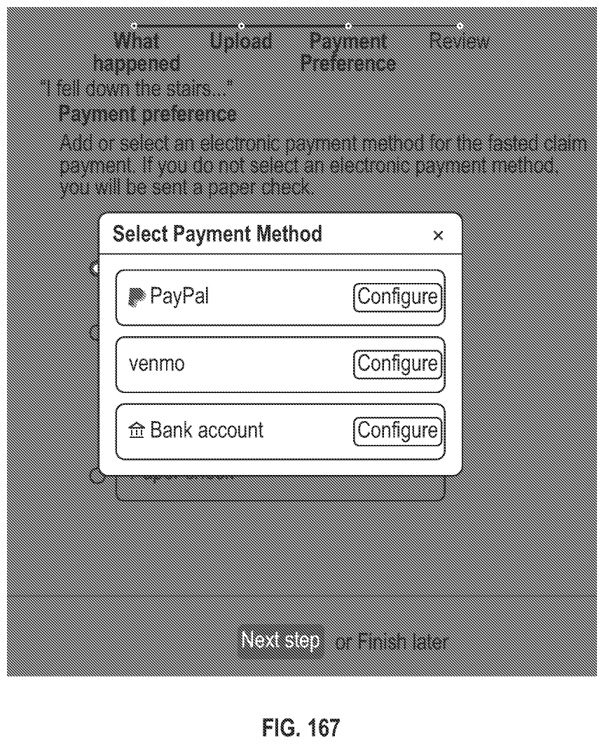

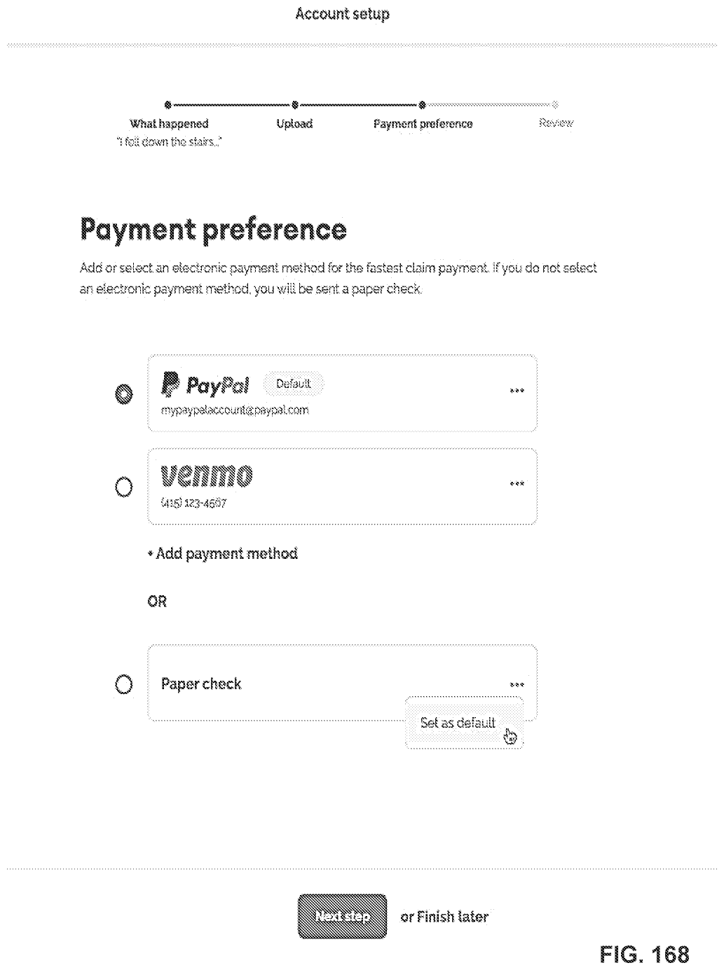

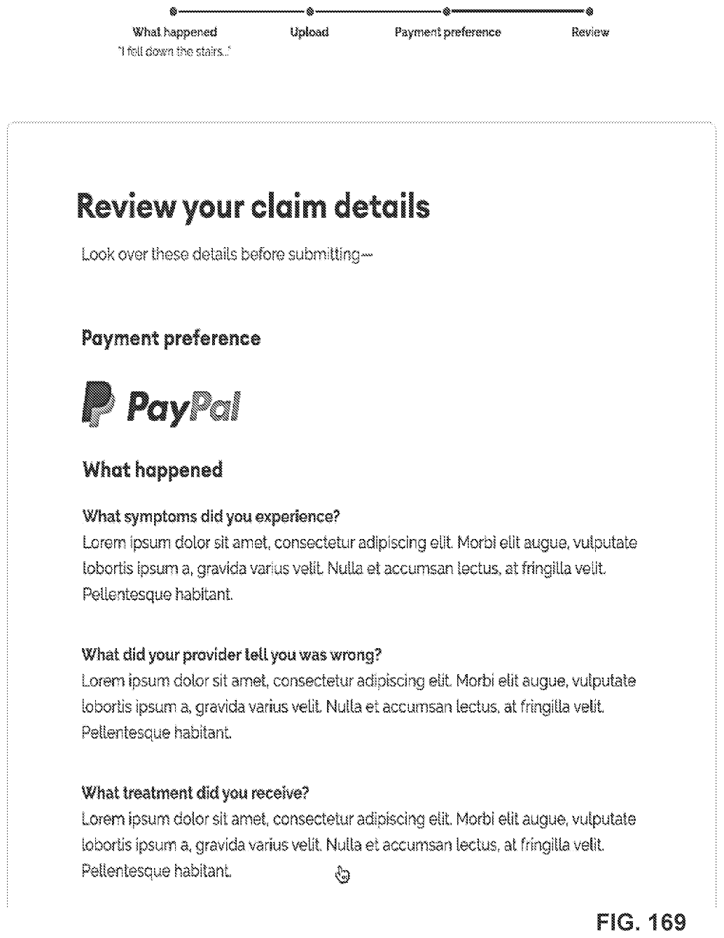

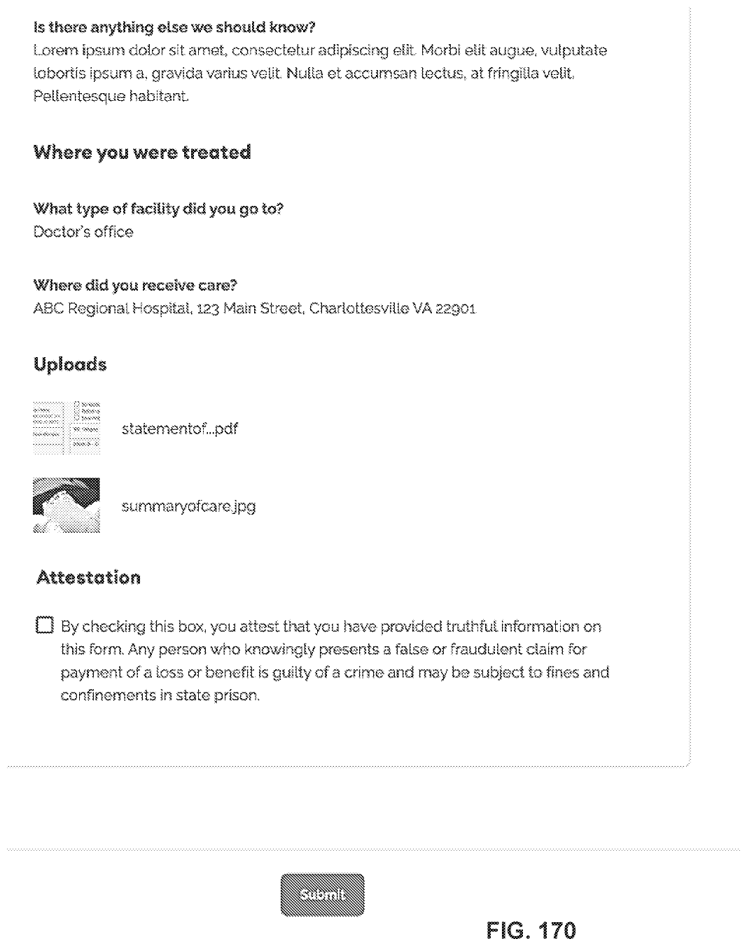

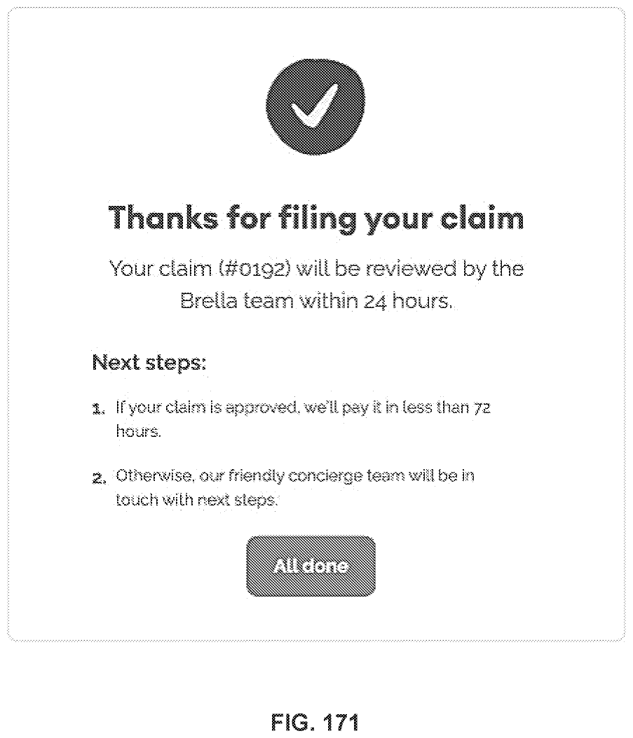

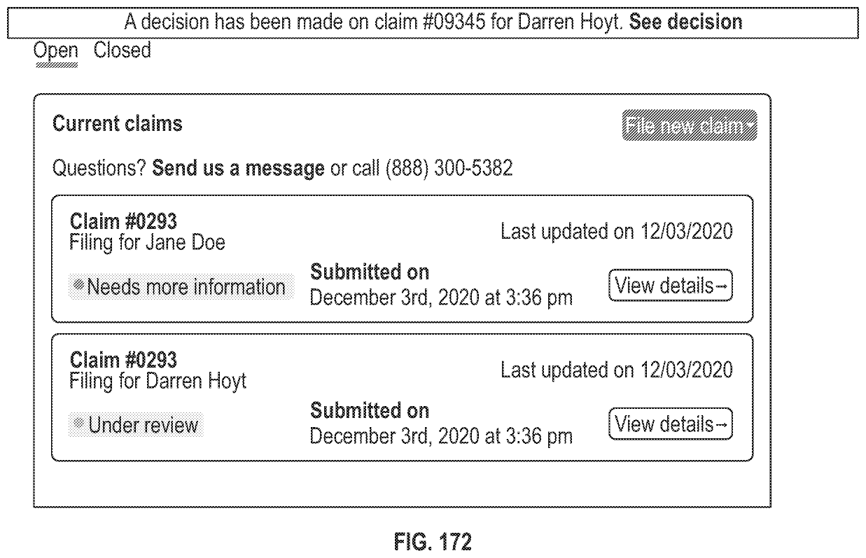

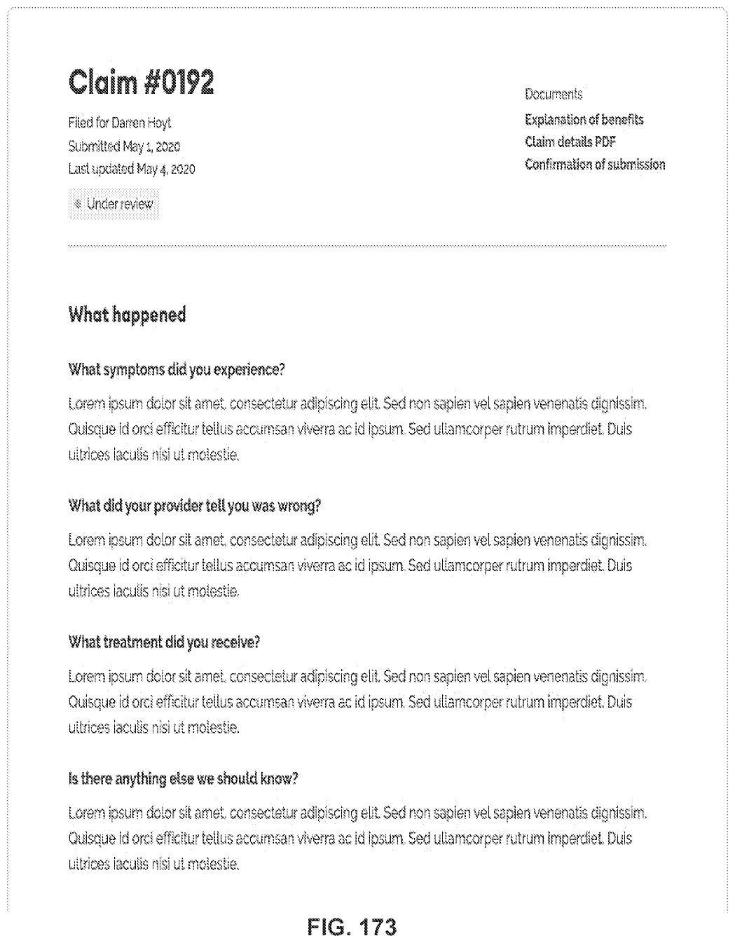

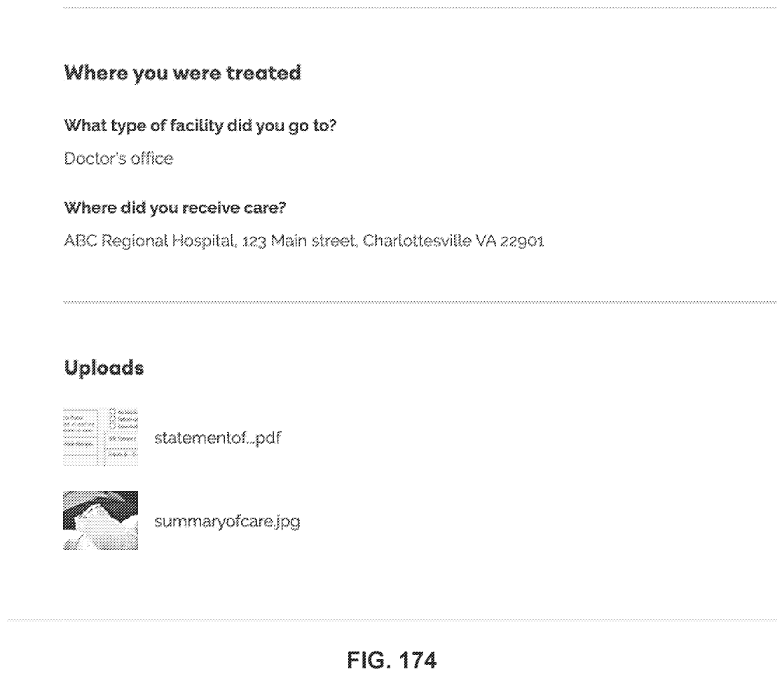

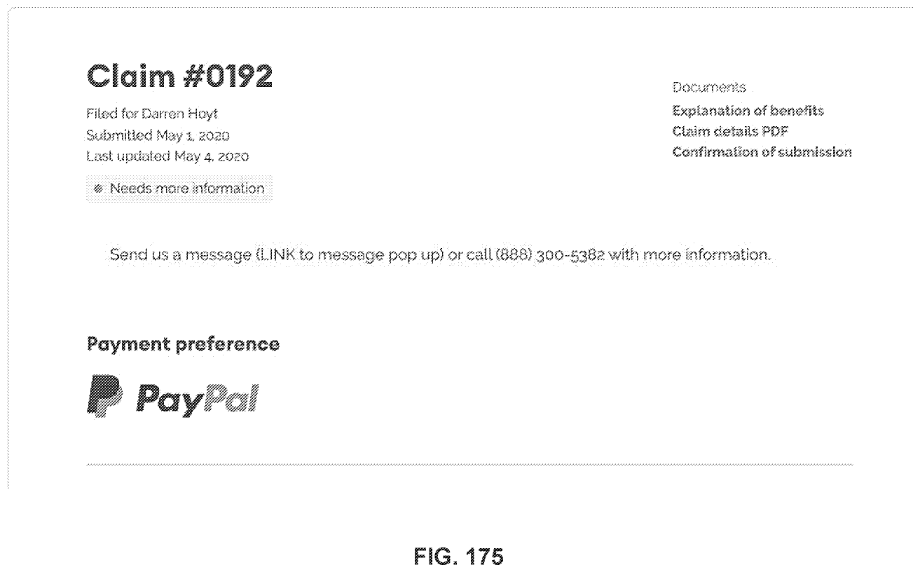

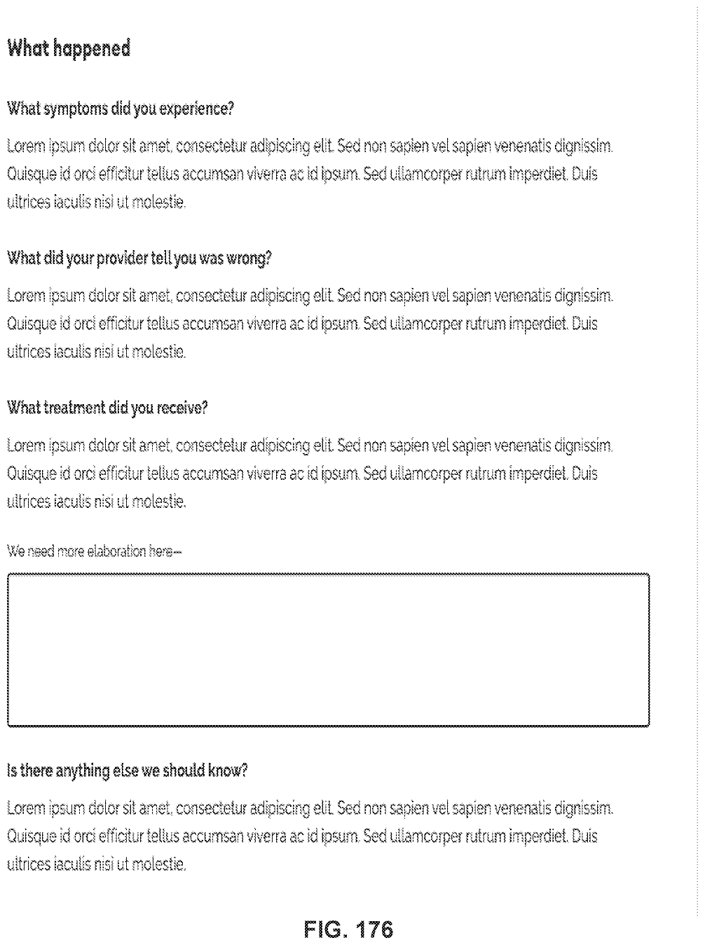

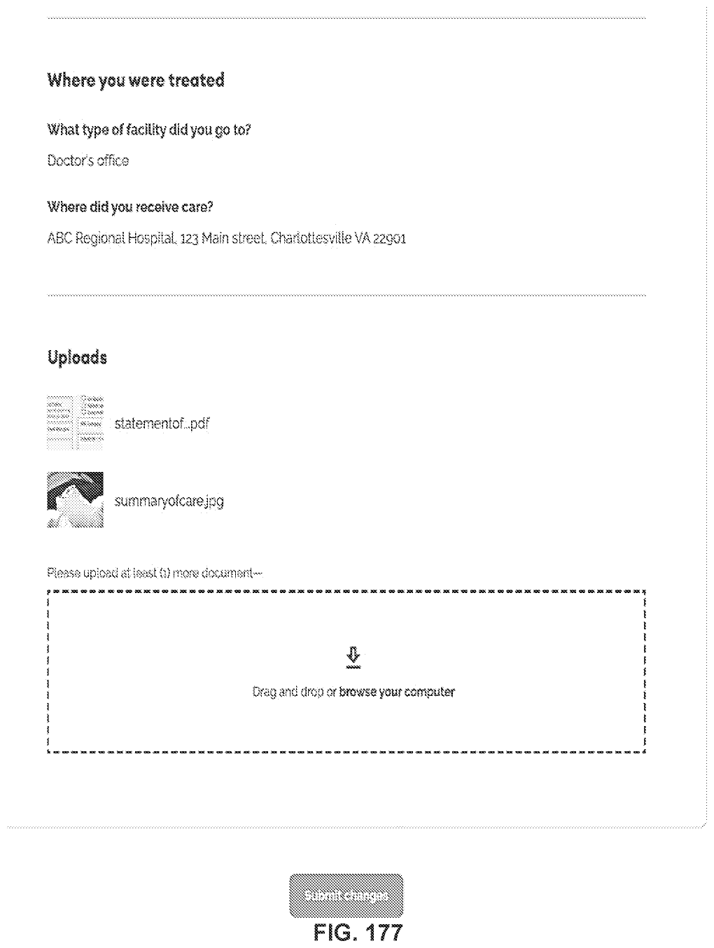

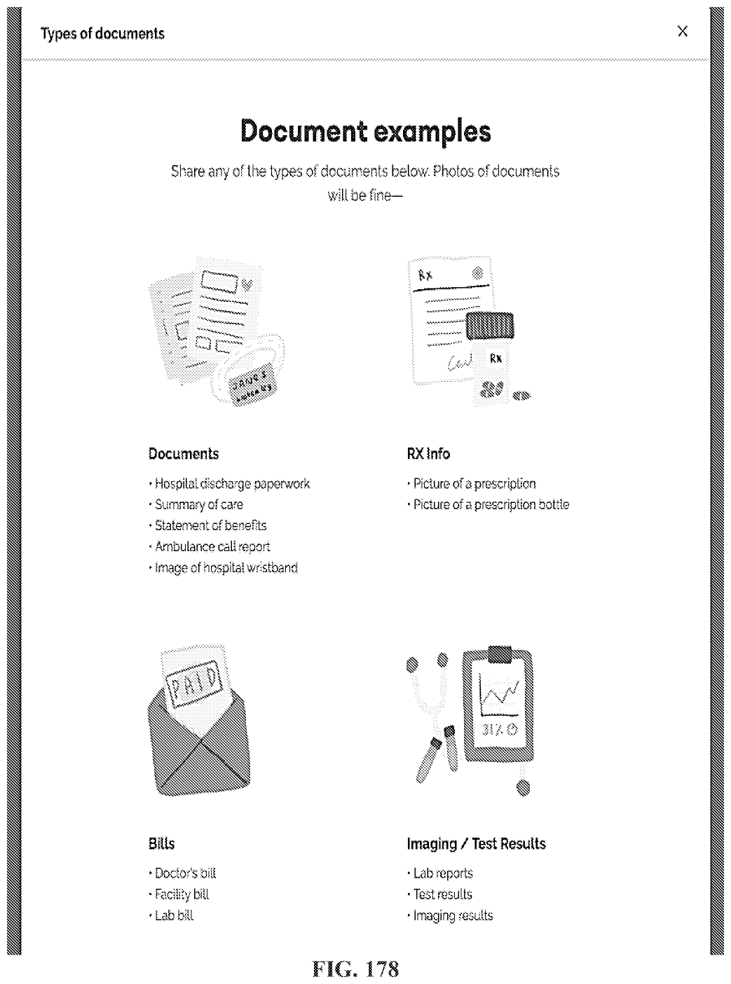

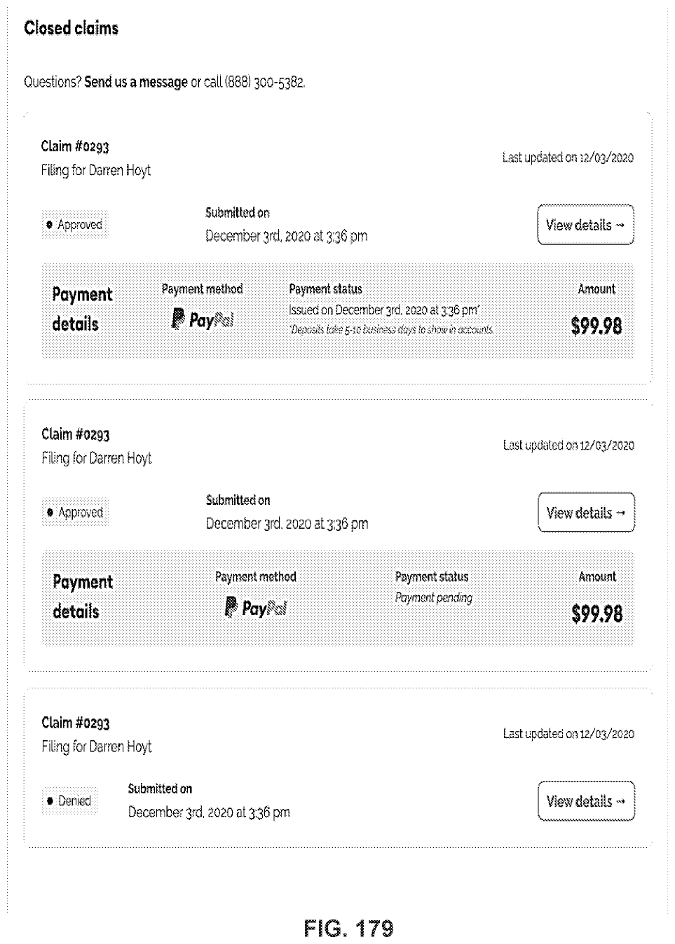

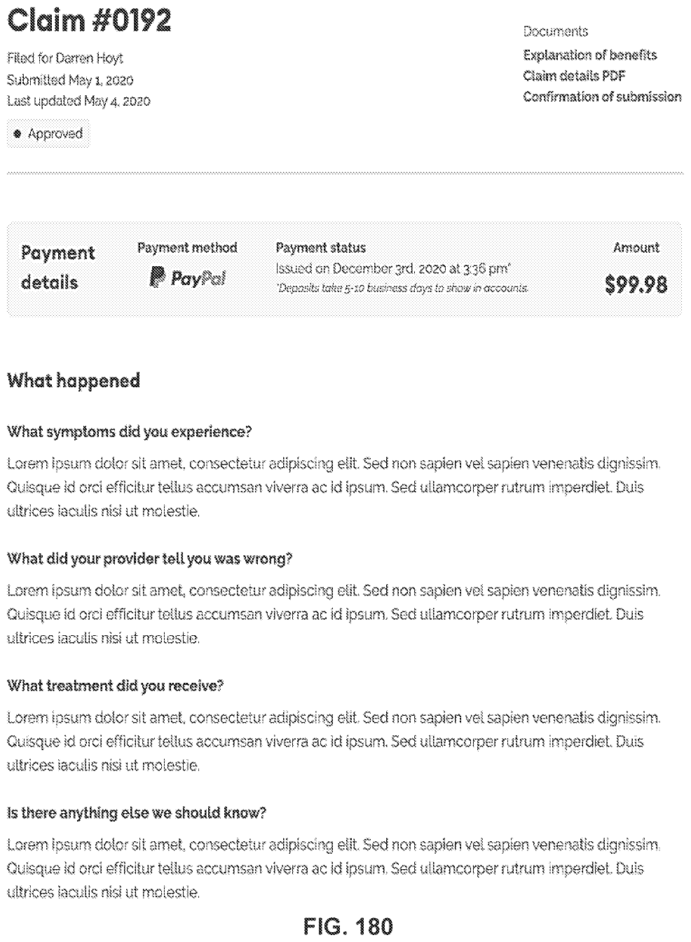

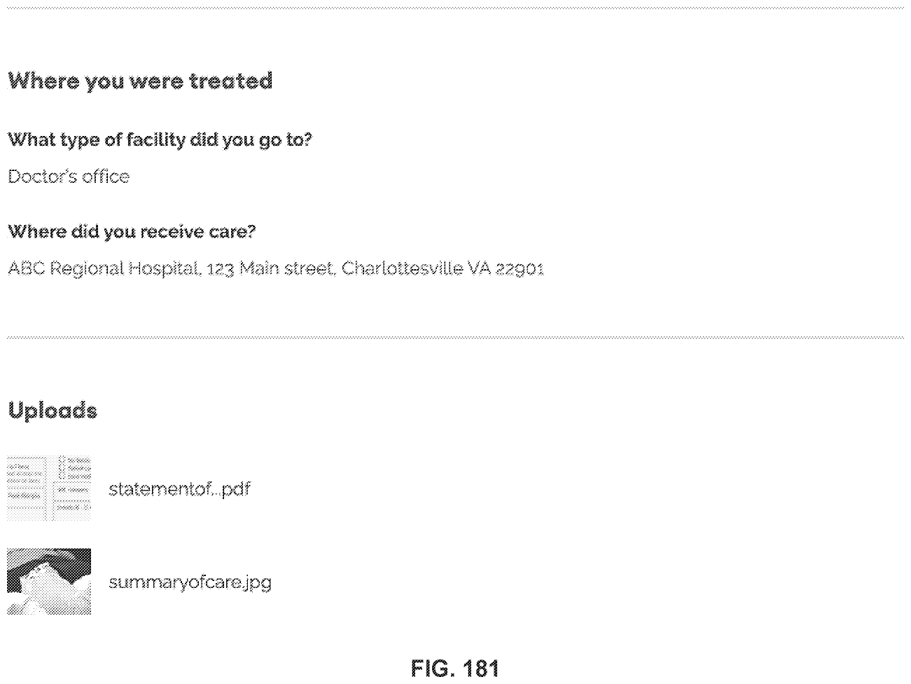

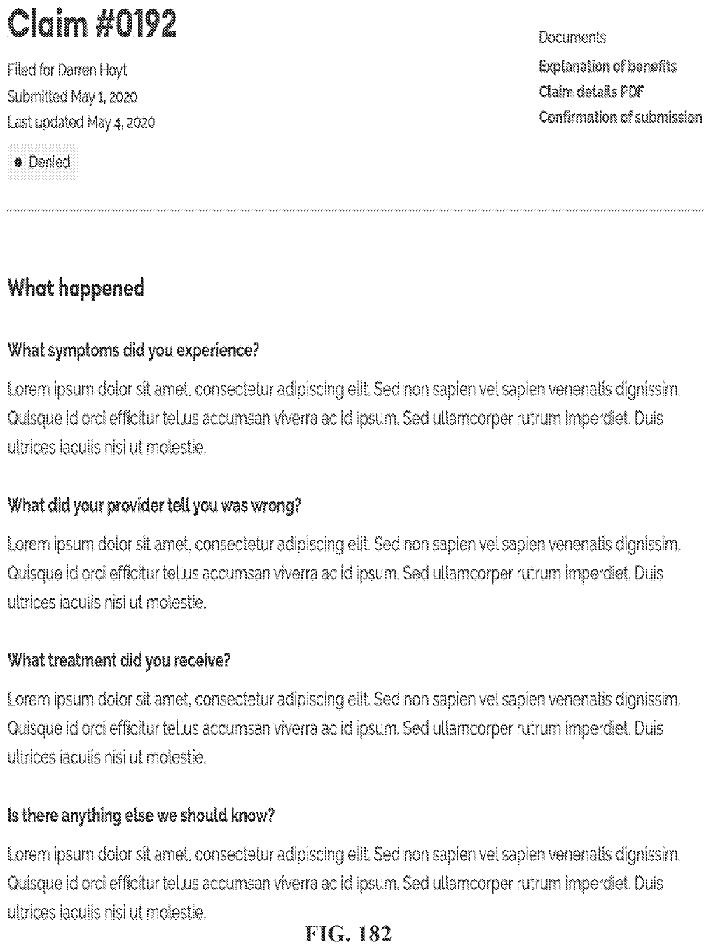

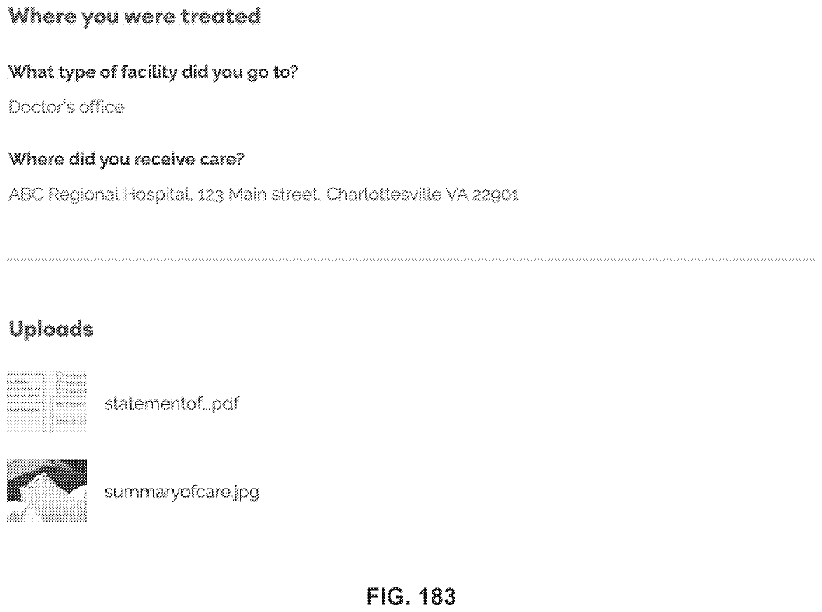

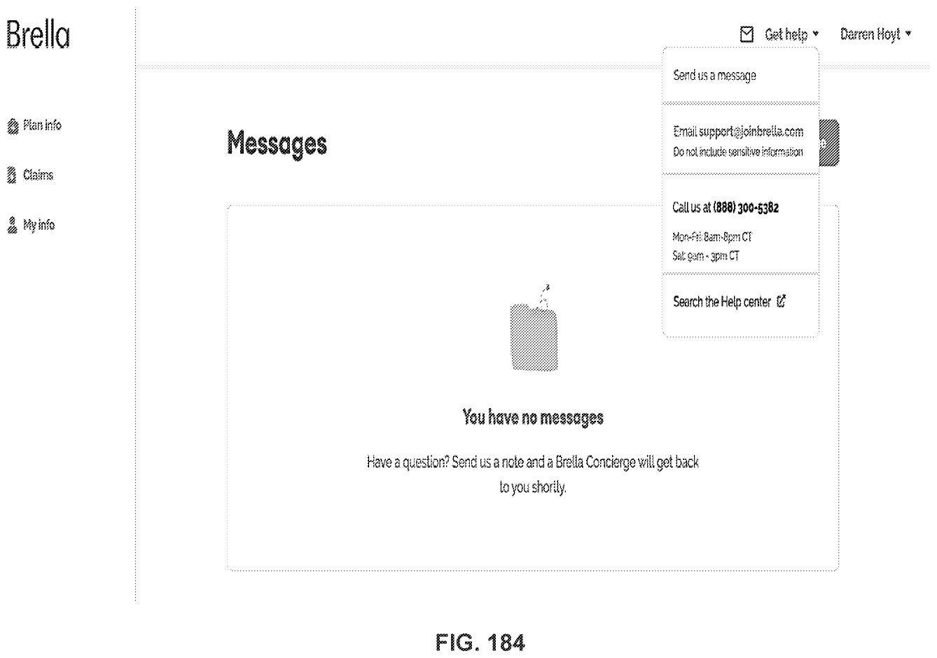

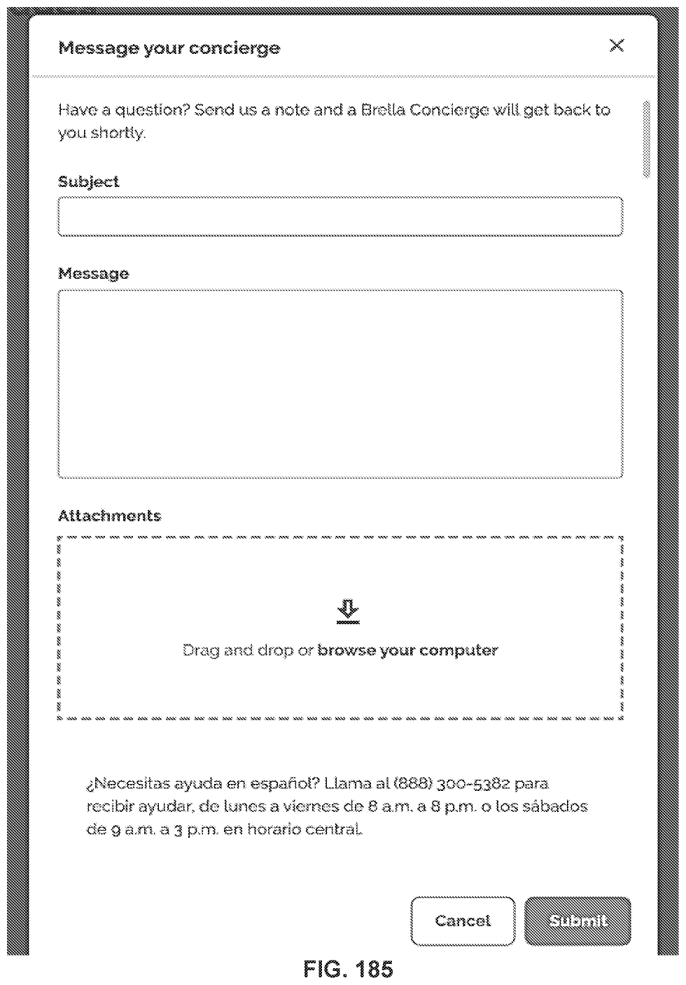

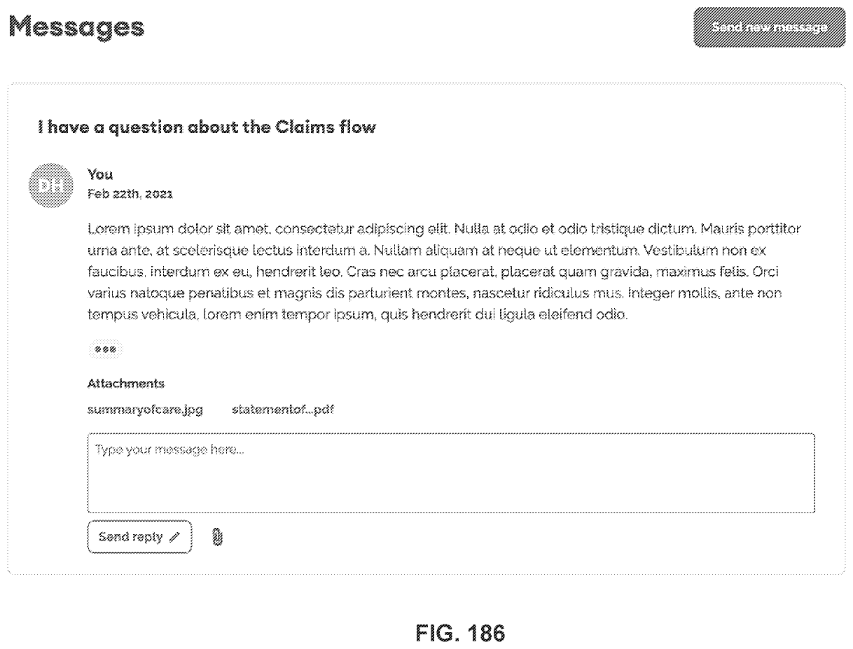

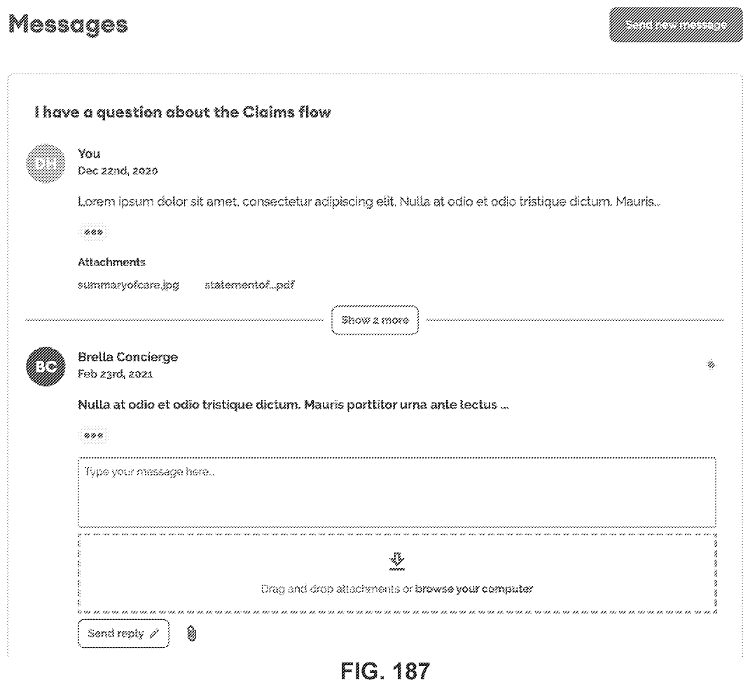

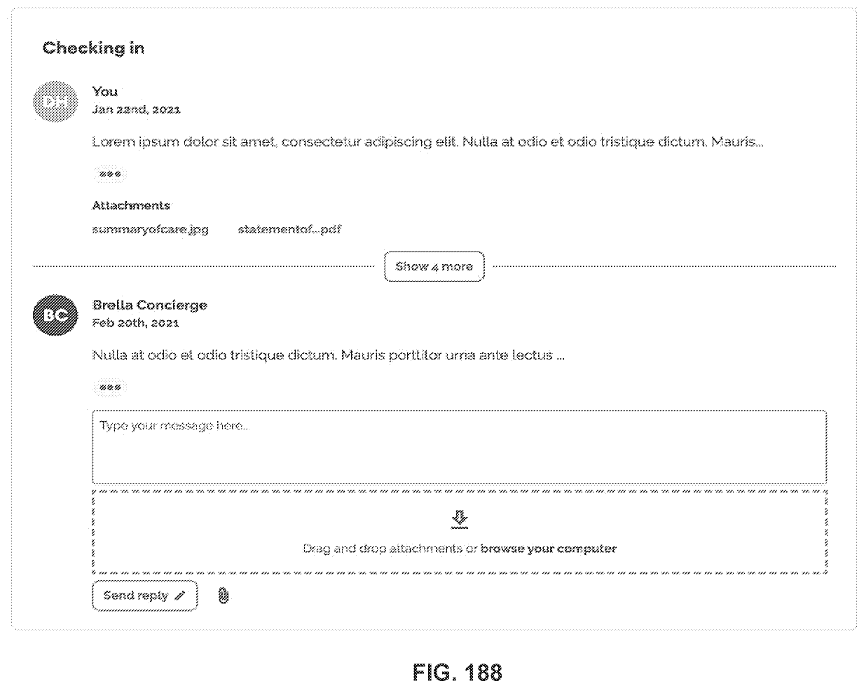

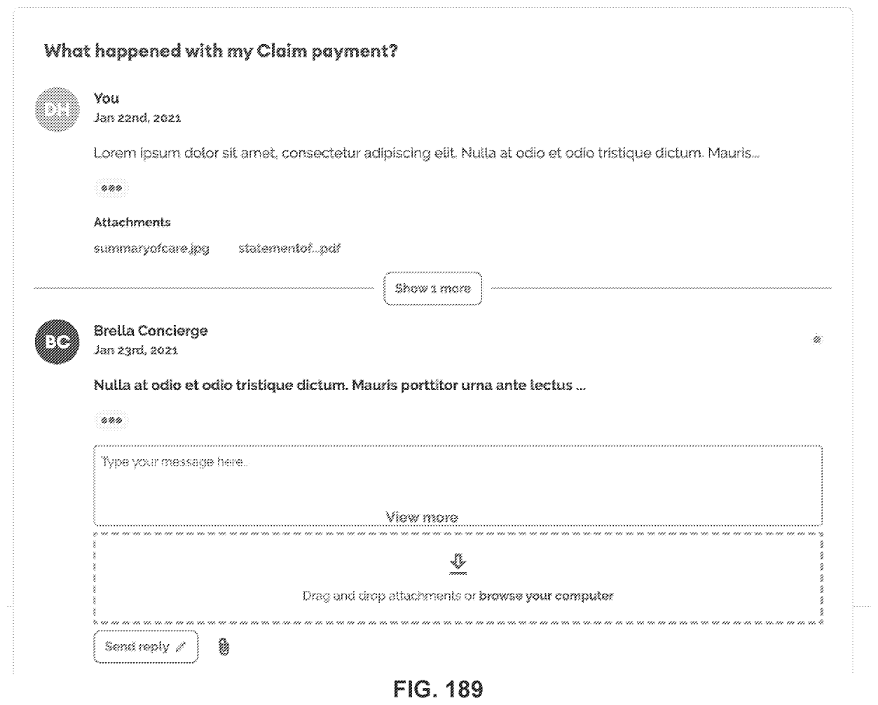

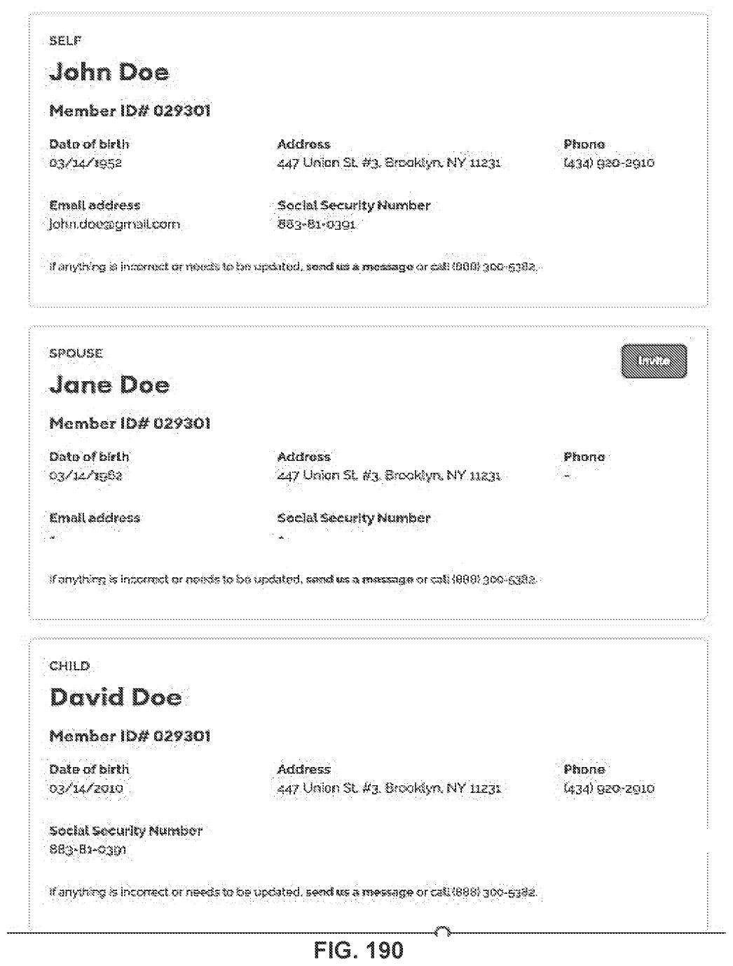

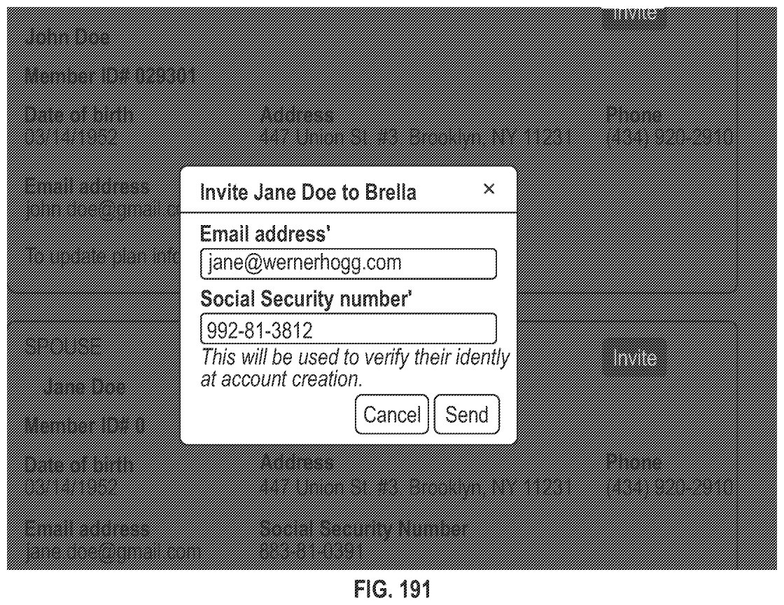

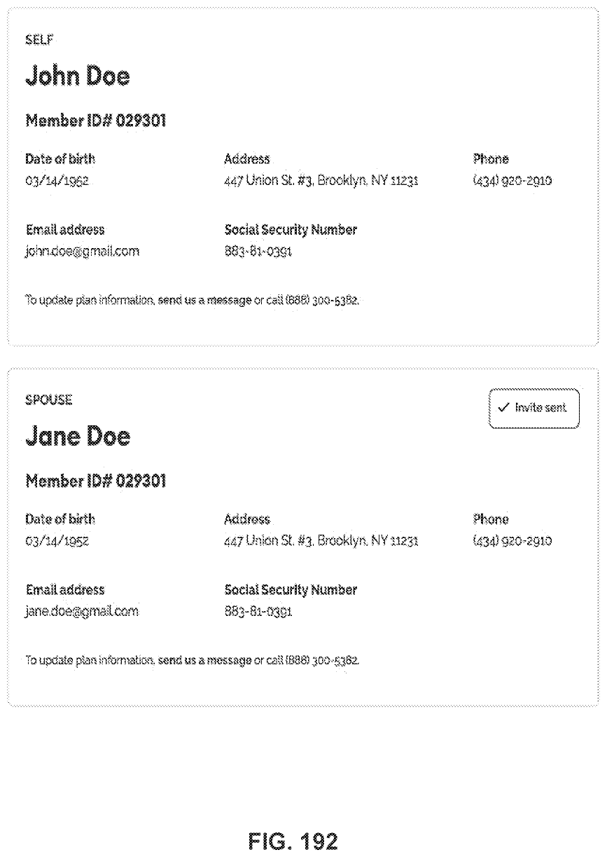

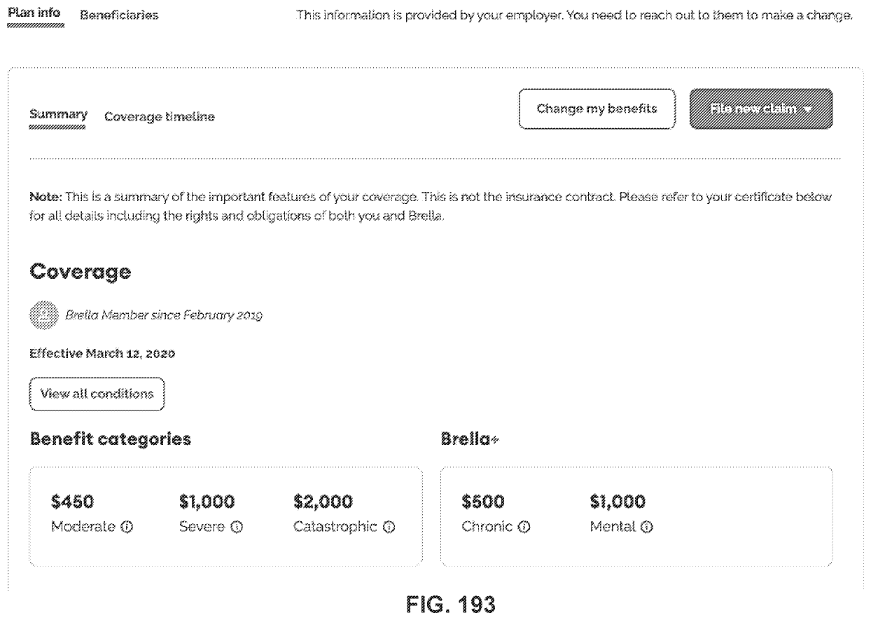

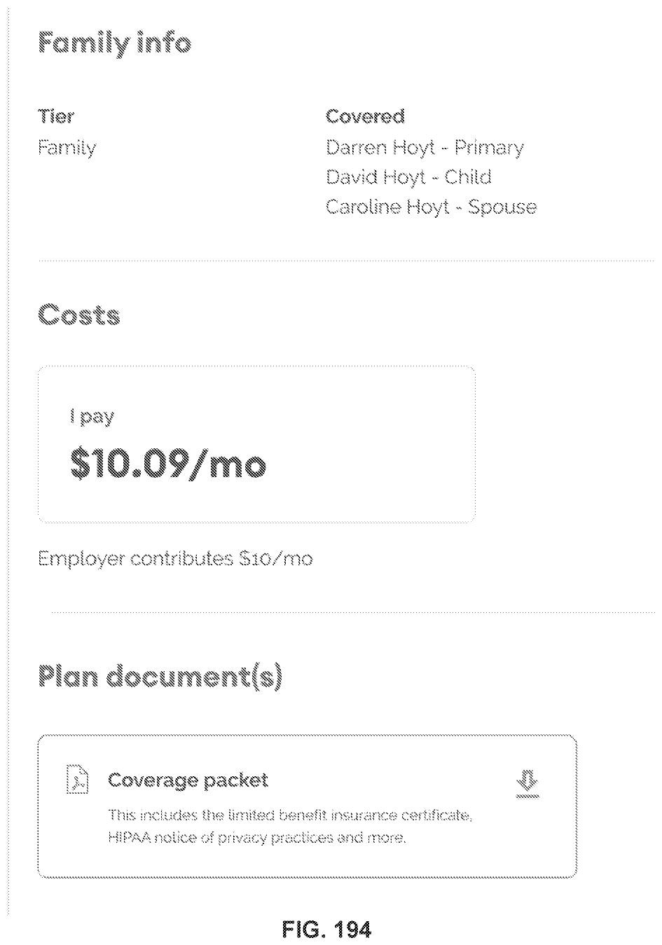

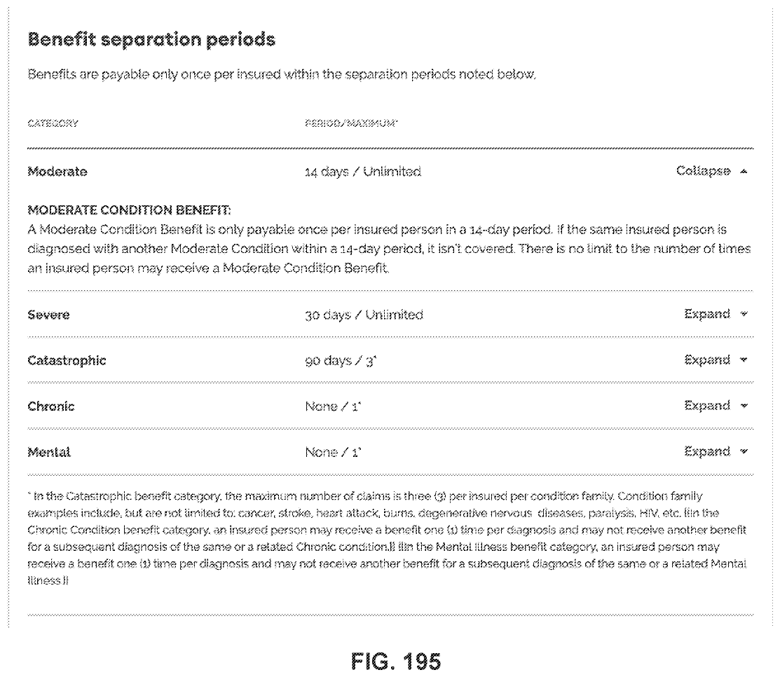

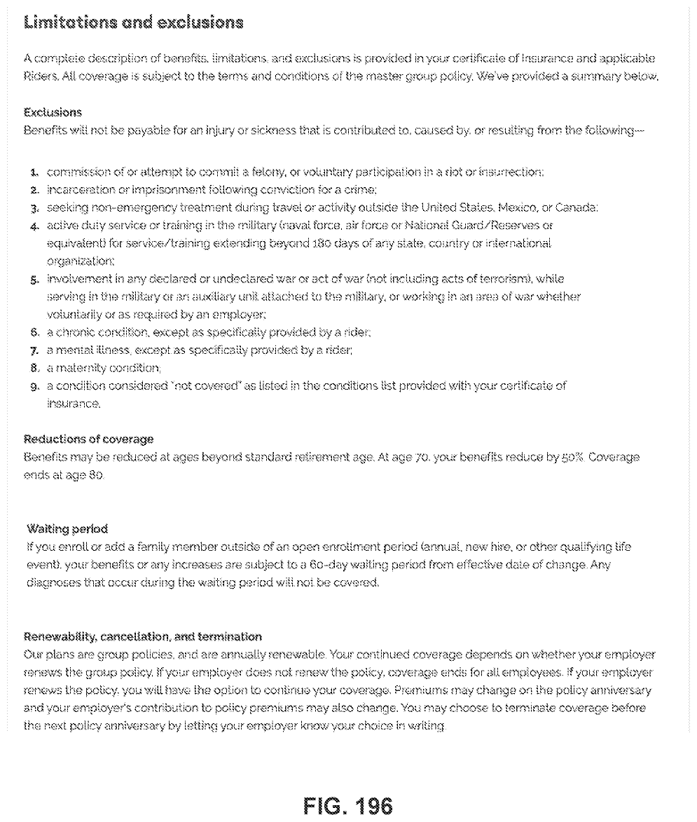

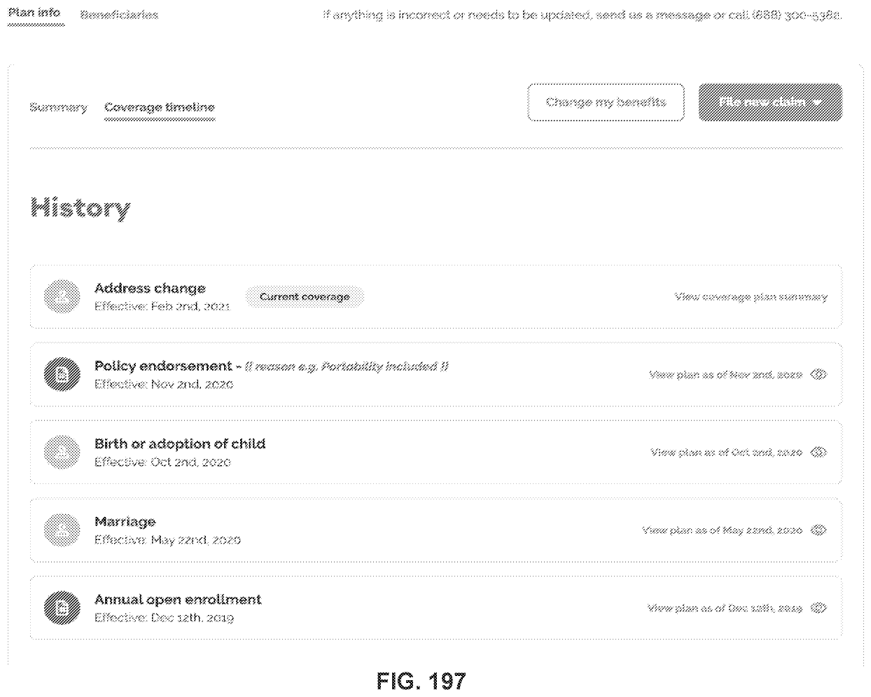

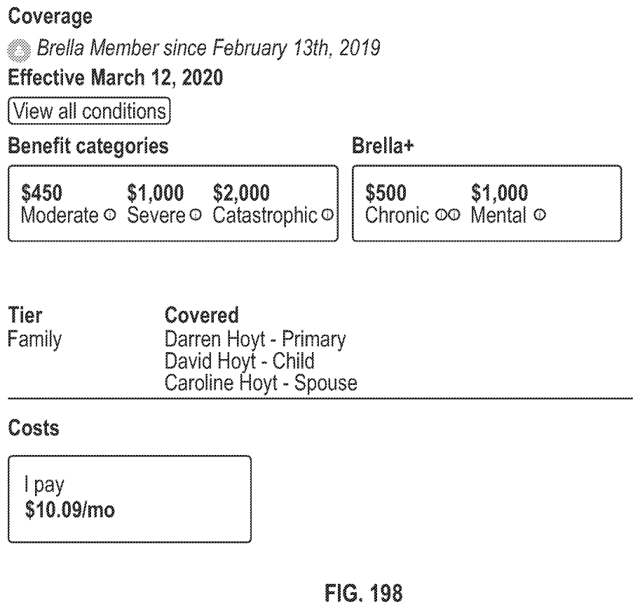

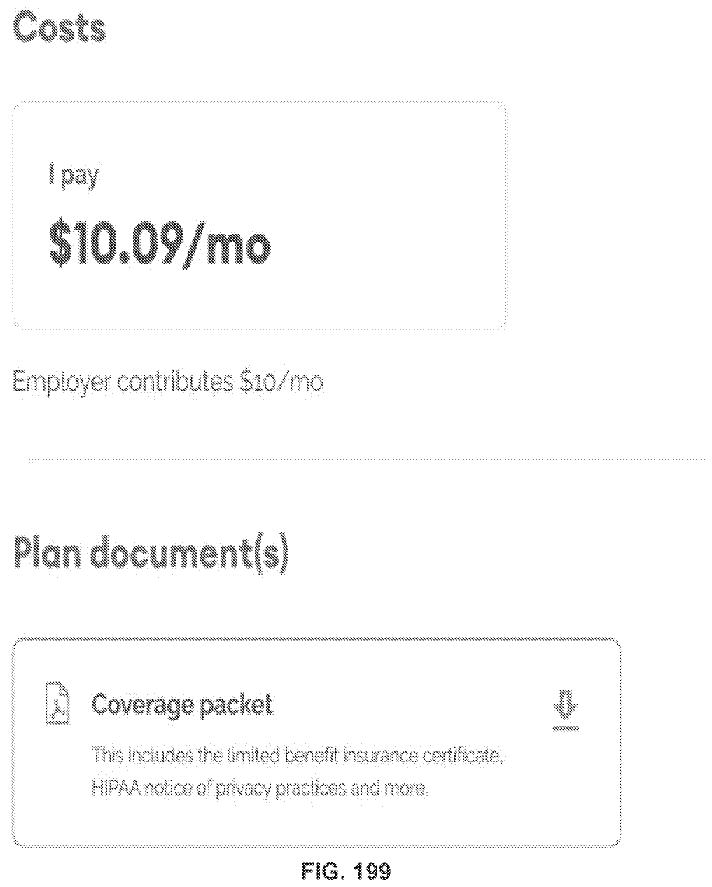

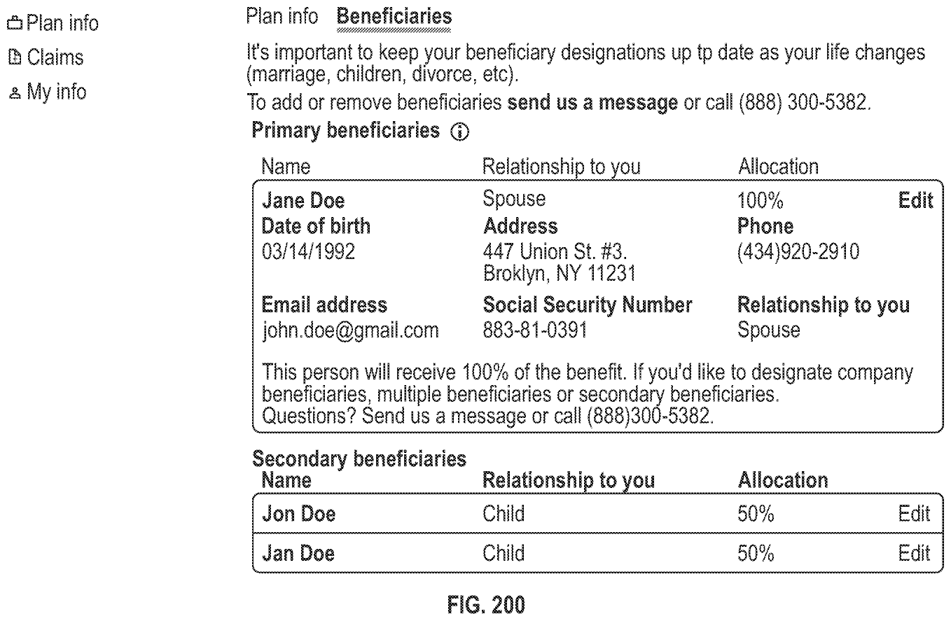

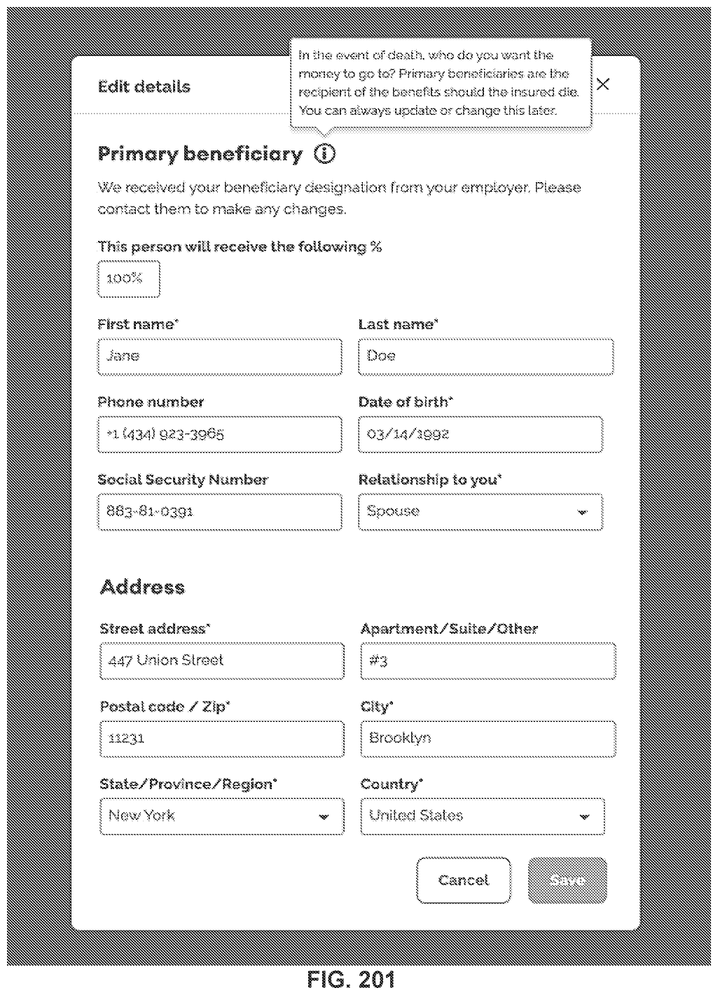

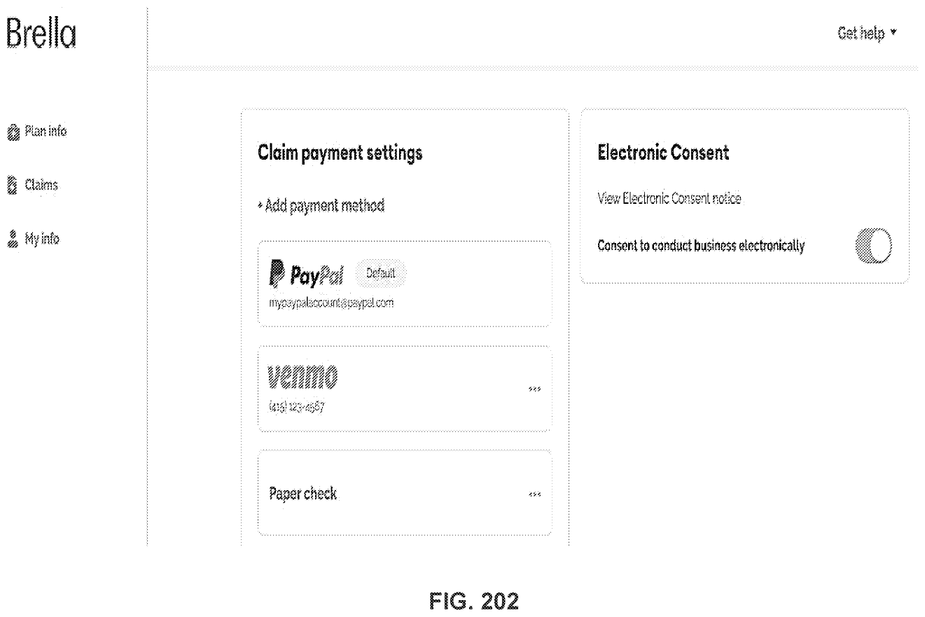

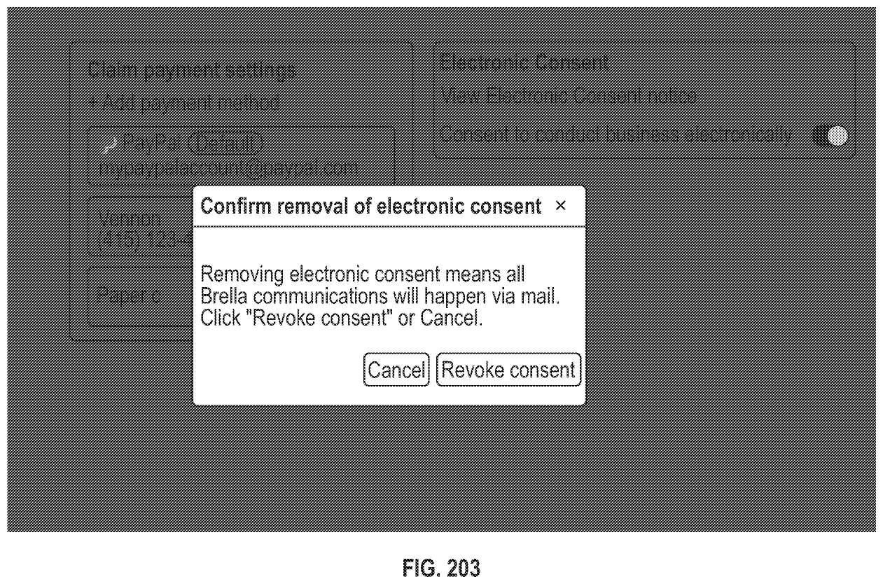

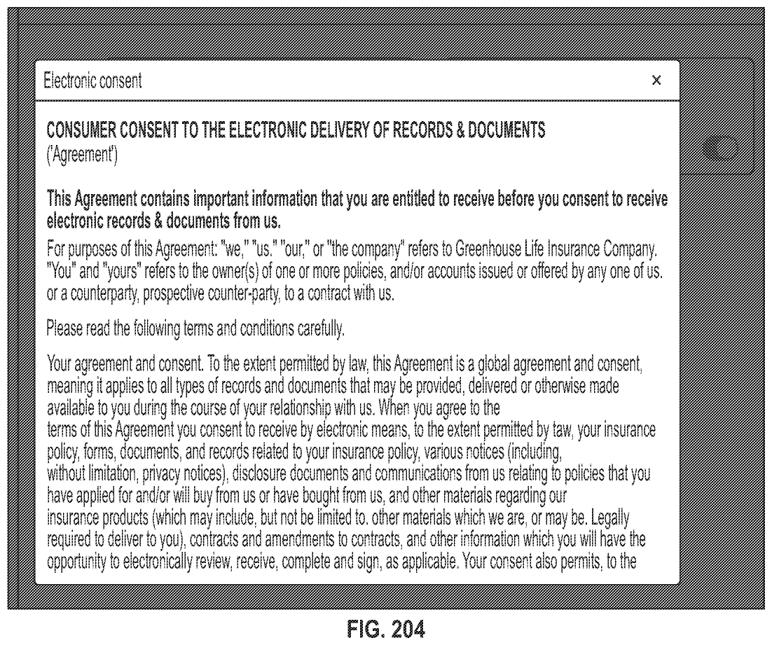

As shown in FIG. 18, in some embodiments, the system includes a combination of one or more modules, platforms, and engines described herein. However, each of the one or more modules, platforms, and engines described herein are also their own system and each comprise functionality (e.g., APIs, software to software interfaces) that allow them not only to interact with each other, but with third party systems as well according to some embodiments. In some embodiments, the interaction with third party system is through the secure processing platform. In some embodiments, one or more modules can directly interface and/or be imbedded into a third party platform. In this respect, each of the one or more modules, platforms, and engines described herein are configured to be integrated into third party systems without integrating one or more of the other modules, platforms, engines, and/or the system as a whole according to some embodiments. As a non-limiting example, in some embodiments the recommendation engine may be integrated into a third-party insurance company's platform to increase the probability a potential member will accept a presented package. In some embodiments, as another non-limiting example, a third-party insurance company may integrate the claims module into their platform to obtain the benefit of automatic claim evaluation and payout. Third-party insurance companies may also choose to integrate the underwriting module to obtain the benefit of automation and more accurate risk analysis according to some embodiments. Therefore, it is understood that all embodiments presented herein are compatible with each other, and that each embodiment is integratable with third-party systems by itself or in conjunction with one or more portions of other embodiments. In some embodiments, the ability to integrate with third-party systems enables a co-branded version of the system to be presented to a potential member. In some embodiments, revenues from the co-branded system can be generated by (a) a percentage of gross premiums and/or (b) the ability to participate in risk via reinsurance capacity.

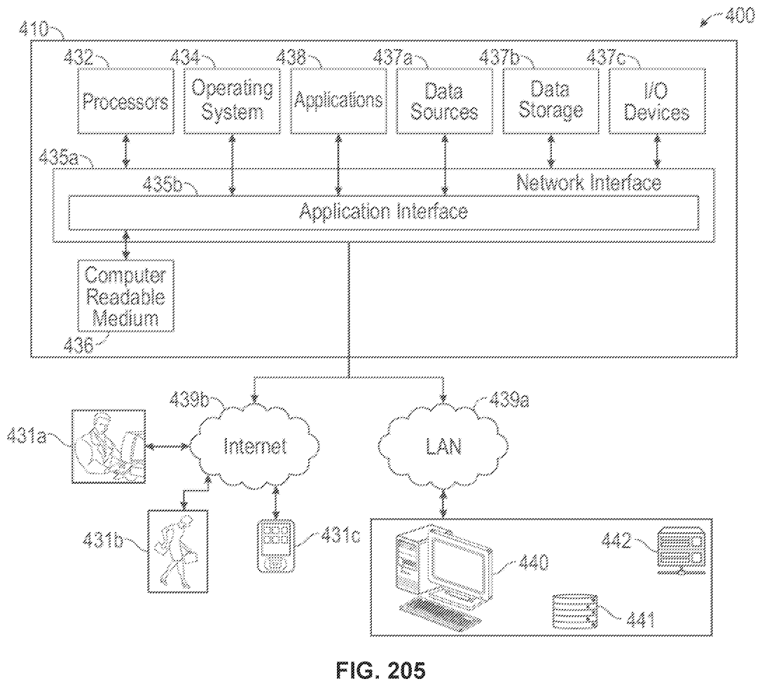

FIG. 205 illustrates a computer system 410 enabling or comprising the systems and methods in accordance with some embodiments of the system. In some embodiments, the computer system 410 can operate and/or process computer-executable code of one or more software modules of the aforementioned system and method. Further, in some embodiments, the computer system 410 can operate and/or display information within one or more graphical user interfaces (e.g., Human-Machine Interfaces (“HMIs”)) integrated with or coupled to the system.

In some embodiments, the computer system 410 can comprise at least one processor 432. In some embodiments, at least one processor 432 can reside in, or coupled to, one or more conventional server platforms (not shown). In some embodiments, the computer system 410 can include a network interface 435a and an application interface 435b coupled to the least one processor 432 capable of processing at least one operating system 434. Further, in some embodiments, the interfaces 435a, 435b coupled to at least one processor 432 can be configured to process one or more of the software modules (e.g., such as enterprise applications 438). In some embodiments, the software application modules 438 can include server-based software and can operate to host at least one user account and/or at least one client account and operate to transfer data between one or more of these accounts using the at least one processor 432.

With the above embodiments in mind, it is understood that the system can employ various computer-implemented operations involving data stored in computer systems. Moreover, the above-described databases and models described throughout this disclosure can store analytical models and other data on computer-readable storage media within the computer system 410 and on computer-readable storage media coupled to the computer system 410 according to various embodiments. In addition, in some embodiments, the above-described applications of the system can be stored on computer-readable storage media within the computer system 410 and on computer-readable storage media coupled to the computer system 410. In some embodiments, these operations are those requiring physical manipulation of physical quantities. Usually, though not necessarily, in some embodiments these quantities take the form of one or more of electrical, electromagnetic, magnetic, optical, or magneto-optical signals capable of being stored, transferred, combined, compared, and otherwise manipulated. In some embodiments, the computer system 410 can comprise at least one computer readable medium 436 coupled to at least one of at least one data source 437a, at least one data storage 437b, and/or at least one input/output 437c. In some embodiments, the computer system 410 can be embodied as computer readable code on a computer readable medium 436. In some embodiments, the computer readable medium 436 can be any data storage that can store data, which can thereafter be read by a computer (such as computer 440). In some embodiments, the computer readable medium 436 can be any physical or material medium that can be used to tangibly store the desired information or data or instructions and which can be accessed by a computer 440 or processor 432. In some embodiments, the computer readable medium 436 can include hard drives, network attached storage (NAS), read-only memory, random-access memory, FLASH based memory, CD-ROMs, CD-Rs, CD-RWs, DVDs, magnetic tapes, and other optical and non-optical data storage. In some embodiments, various other forms of computer-readable media 436 can transmit or carry instructions to a remote computer 440 and/or at least one user 431, including a router, private or public network, or other transmission or channel, both wired and wireless. In some embodiments, the software application modules 438 can be configured to send and receive data from a database (e.g., from a computer readable medium 436 including data sources 437a and data storage 437b that can comprise a database), and data can be received by the software application modules 438 from at least one other source. In some embodiments, at least one of the software application modules 438 can be configured within the computer system 410 to output data to at least one user 431 via at least one graphical user interface rendered on at least one digital display.

In some embodiments, the computer readable medium 436 can be distributed over a conventional computer network via the network interface 435a where the system embodied by the computer readable code can be stored and executed in a distributed fashion. For example, in some embodiments, one or more components of the computer system 410 can be coupled to send and/or receive data through a local area network (“LAN”) 439a and/or an internet coupled network 439b (e.g., such as a wireless internet). In some embodiments, the networks 439a, 439b can include wide area networks (“WAN”), direct connections (e.g., through a universal serial bus port), or other forms of computer-readable media 436, or any combination thereof.

In some embodiments, components of the networks 439a, 439b can include any number of personal computers 440 which include for example desktop computers, and/or laptop computers, or any fixed, generally non-mobile internet appliances coupled through the LAN 439a. For example, some embodiments include one or more of personal computers 440, databases 441, and/or servers 442 coupled through the LAN 439a that can be configured for any type of user including an administrator. Some embodiments can include one or more personal computers 440 coupled through network 439b. In some embodiments, one or more components of the computer system 410 can be coupled to send or receive data through an internet network (e.g., such as network 439b). For example, some embodiments include at least one user 431a, 431b, is coupled wirelessly and accessing one or more software modules of the system including at least one enterprise application 438 via an input and output (“I/O”) 437c. In some embodiments, the computer system 410 can enable at least one user 431a, 431b, to be coupled to access enterprise applications 438 via an I/O 437c through LAN 439a. In some embodiments, the user 431 can comprise a user 431a coupled to the computer system 410 using a desktop computer, and/or laptop computers, or any fixed, generally non-mobile internet appliances coupled through the internet 439b. In some embodiments, the user can comprise a mobile user 431b coupled to the computer system 410. In some embodiments, the user 431b can connect using any mobile computing 431c to wireless coupled to the computer system 410, including, but not limited to, one or more personal digital assistants, at least one cellular phone, at least one mobile phone, at least one smart phone, at least one pager, at least one digital tablets, and/or at least one fixed or mobile internet appliances.

In some embodiments, the system includes a journey workflow (i.e., “the journey”). In some embodiments, FIG. 18 shows an example journey workflow starting from the partner portal and going clockwise around the secure processing platform to the member portal module. In some embodiments, the secure processing platform (which could also be a third party platform) interfaces with one or more modules to create a customizable module system according to some embodiments. In some embodiments, one or more modules are configured to execute all associated steps and/or functionality, while in some embodiments a central system is configured to execute at least part of the disclosed steps and/or functionality. The system shown in FIG. 18 and steps in other associated figures according to some embodiments are non-limiting, and it is understood that the system can comprise any module described herein. In some embodiments, the journey includes one or more of a partner, sales and onboarding process. In some embodiments, the system includes a partner portal module configured to generate one or more graphical user interfaces to work with partners and train them.

In some embodiments, the partner module includes one or more graphical user interfaces to present products and proposals to employers. In some embodiments, the partner module includes an appointment flow to submit an appointment request. In some embodiments, the system (e.g., partner module, secure processing platform) is configured to then create an appointment as needed.

In some embodiments, the system provides partners access to a partner portal GUI (any reference to a “portal” herein is also a reference to an associated system generated GUI). In some embodiments, the partner portal module includes quote requests and employer management. In some embodiments, the partner portal includes one or more abilities to create a quote, the ability to see quotes, the ability to review proposals, and/or the ability to access employers the contract details of employers who are in force. In some embodiments, the access is over a secure connection.

In some embodiments, the system is configured to submit a request through the partner portal to an underwriting platform. In some embodiments, the system is configured to integrate with a third-party co-branded portal through an API. In some embodiments, after a quote request, it goes through an underwriting platform (module). In some embodiments, after it goes through underwriting and rate calculation, a proposal delivery module sends the proposal to the partner and the employer. In some embodiments, the proposal is a customized interactive proposal that both partners and employers can experiment with and test options before they accept it.

In some embodiments, after the employer sales process, the journey workflow continues to implementation and case install. In some embodiments, the system is configured to support the partner however they need, and in some embodiments the system is configured to make it so that the employers and/or the partner can go through the whole process without interacting with a human if desired. In some embodiments, the system is focused on the automation of the workflow.

In some embodiments, the system includes a proposal acceptance graphical user interface. In some embodiments, from the proposal acceptance interface the employer account is automatically created. In some embodiments, the employer account is automatically created for access to their portal. In some embodiments, the system is configured to enable the group application signature through an integrated electronic signature (e.g., DocuSign®) experience. In some embodiments, the policy and the certificate documents are automatically generated and made available as a result of the signature of the group application and then the employer's open enrollment implementation can then be completed. In some embodiments, proposal acceptance includes a GUI generated by a computer application. In some embodiments, one or more documents are transferred over an encrypted connection.

In some embodiments, the system is configured to generate the group application, policy, and certificate documents for the employer as the next step in the journey workflow. In some embodiments, the system includes three different versions of an implementation form for scenarios where the employer is using the system's enrollment platform. In some embodiments, those different versions are based on the enrollment platform (Brella Enroll or a BenAdmin platform), the employer contribution strategy, as well as other factors. In some embodiments, the system is configured to implement scenarios where the employer is using a benefits administration (BenAdmin) platform or scenarios where it is one hundred percent employer paid. In some embodiments, the system will not implement a shopping experience and, instead, an implementation file is received. In some embodiments, all electronic documents are submitted online and generated automatically based on the contract provisions and the proposal details.

In some embodiments, the journey workflow includes full post-enrollment workflows for members using one or more of web and mobile interfaces. In some embodiments, the core tenets and biggest touch points are ongoing member engagement. In some embodiments, as described previously, the system provides an effective way to stay top of mind for people and make claims easy, both from a submission perspective and a payment perspective.

In some embodiments, the system includes billing functionality. In some embodiments, employers have access to an ongoing human resources administration type of employer portal provided by the system. In some embodiments, the employer portal enables employers to review employees' information, run reports, access their bill, preview their bill, as well as access any conventional information including, as a non-limiting example, electronic payments. In some embodiments, the system focuses on billing simplicity for the employer, focuses on ways that may eliminate the need of retroactive adjustments, and focuses on ways to facilitate an easier reconciliation process when it comes to reconciling (i.e., reconciling any differences between what is in a payroll report or what is in the system). In some embodiments, the system includes a direct bill module configured to bill employees directly (i.e., not via a payroll deduction with the employer).

In some embodiments, the journey workflow includes an enrollment shopping experience. In some embodiments, the enrollment shopping experience includes an enrollment platform that the system provides to employers that do not have an online enrollment tool. In some embodiments, the platform is used for initial annual open enrollment. In some embodiments, it is also used for those employers for ongoing Qualified Life Event (“QLE”) management throughout the year, annual open enrollment in following years, so it is not a one-time annual open enrollment tool. In some embodiments, if a new hire was added through the employer portal, this functionality would add them as well.

In some embodiments, the system takes a different approach to education and engagement. In some embodiments, the system includes a more interactive experience that catches a user's attention and keeps them engaged as they are learning about the product and as the system provides layers of building blocks of information. In some embodiments, the system is conversational in terms of feedback and information presentation providing a give-and-take experience: a “you tell us something, we tell you something” approach provided by the system keeps the user engaged. In some embodiments, the system is configured to provide information in smaller, consumable pieces.

In some embodiments, the enrollment platform UI starts with a welcome message. In some embodiments, the system is configured to send out open enrollment e-mails to all the employees and direct employees to enrollment by, as a non-limiting example, clicking on a link in an e-mail. In some embodiments, a validation step includes entering a birthday. In some embodiments, the system is configured to provide feedback using language that's common for consumers that feels very conversational both to be approachable but also to truly help them understand.

In some embodiments, the enrollment platform includes tips or deep dive areas throughout the platform. In some embodiments, the system is configured to assist a lot of different buyer types, including, as non-limiting examples, people who understand instantly, those who do not want help, those who just want to breeze through and make their election, those who are already convinced, and those who just want to purchase the product and check out, to people who are the opposite end of the spectrum where they want to read as much detail as is made available to them. The system provides a flow that a user can go through quickly, but a user can also take their time and go through different areas of the system, dive into all of those different content pieces, and gather whatever they need out of it. Some embodiments provide interfaces and materials in a wide variety of languages.

In some embodiments, the next step is for the system to ask the consumer a little bit about themselves. This will funnel into the ultimate recommendation that the system provides to them. In some embodiments, based on what the consumer just selected, the system can customize the product to the consumer's particular needs.

In some embodiments, the system is configured to funnel the information into the recommendation that the system provides to them via the recommendation engine as previously described. In some embodiments, the system is configured to emphasize the breadth of covered conditions during the enrollment. In some embodiments, the system is configured to emphasize that the claims process is easy and emphasize the presence of support standing by in the form of a customer care team.

In some embodiments, the enrollments platform is configured to provide information in the form of stories involving individuals such as the non-limiting examples previously presented. In some embodiments, the system provides an interface for a user to personalize their plans. In some embodiments, the system is configured to ask further questions to assist the recommendation engine. In some embodiments, some non-limiting questions provided by the system include one or more of medical costs and how they feel about the affordability of the recommended plan. In some embodiments, the system is configured to enable a user to skip this if they don't want to answer the questions and still receive a recommendation. In some embodiments, the system is configured to enable a user to enter their budget into the recommendation engine. In some embodiments, the recommendation engine is configured to restrict the recommendation based on affordability.

As previously discussed, in some embodiments, the system includes a Choice Plan. In some embodiments, the system is built to manage, as a non-limiting example, seven increments across each of the three benefit categories and can illustrate pricing through a slider system for employees. In some embodiments, the system is configured to enable an employee to construct their plan selecting a benefit amount for moderate, severe, and catastrophic. In some embodiments, an alternate plan or the other plan that the system provides is a Select Plan, which includes a preconfigured set of options. In some embodiments, the Select Plan includes one or more categories that include value, enhanced and premier (i.e., low, medium, and high). In some embodiments, each category is a preconfigured package. In some embodiments, the plans are integratable into third-party systems as described above.

In some embodiments, based on the increments selected and/or the family tier selected, the system is configured to then enable a user to review the plan details, add personal demographic information and check-out. In some embodiments, following the check-out selection, the system is configured to direct the employee back to the third-party system and/or email their recommendations.

In some embodiments, the system includes a full condition lookup tool. In some embodiments, the conditions lookup tool enables a user to search from all covered conditions and provide them with different types of examples of what's configured. In some embodiments, the system enables employees to look up a summary of their coverage over a secure connection. In some embodiments, some non-limiting examples of available information include costs, plan documents, important details around plans, separation periods, limitations, and exclusions.

In some embodiments, the system includes a claims module as previously described. In some embodiments, the system is configured not to prevent claim submission if incomplete or it hasn't met the number of documents that the system is suggesting. In some embodiments, the system is configured to provide a message that the submission documents have not met the suggested number. In a non-limiting example, if only two documents where three documents are needed, the system would then alert the member that they will be allowed to continue, but that an additional document will make the claim processing faster and more efficient.

In some embodiments, the system includes a member administration platform. In some embodiments, the member administration platform enables an employer to pull up a particular employee and see a list of, as non-limiting examples, their demographic information, their employment related details, their benefit related details, handle a life event, as well as any other conventional insurance information.

In some embodiments, the system includes a billing platform (module). In some embodiments, the system provides the ability to set up payment details through the billing platform. In some embodiments, the system enables a user to preview their invoice which includes a breakdown of totals and all employees included in that preview. In some embodiments, the system is configured to help with reconciliation around flags and retroactive adjustments such as when a member wasn't billed for a portion of their coverage and there is a need to retroactively adjust it. In some embodiments, the system is configured to flag some of the adjustments so that employers have an easier way of understanding what they may have to pay attention to from a one-time difference on a payroll deduction or a catchup, as non-limiting examples. In some embodiments, the billing platform is configured to generate final invoices, enable users to edit their account, add other HR administrators, and run reports as further non-limiting examples of functionality.

The subject matter described herein are directed to technological improvements to the field of computer resource conservation by limiting the computer resources needed to identify, transmit, store, and analyze insurance claims. The disclosure describes the specifics of how a machine including one or more computers comprising one or more processors and one or more non-transitory computers implement the system and its improvements over the prior art. The instructions executed by the machine cannot be performed in the human mind or derived by a human using a pin and paper but require the machine to convert process input data to useful output data. Moreover, the claims presented herein do not attempt to tie-up a judicial exception with known conventional steps implemented by a general-purpose computer; nor do they attempt to tie-up a judicial exception by simply linking it to a technological field. Indeed, the systems and methods described herein were unknown and/or not present in the public domain at the time of filing, and they provide technologic improvements and advantages not known in the prior art. Furthermore, the system includes unconventional steps that confine the claim to a useful application.

It is understood that the system is not limited in its application to the details of construction and the arrangement of components set forth in the previous description or illustrated in the drawings. The system and methods disclosed herein fall within the scope of numerous embodiments. The previous discussion is presented to enable a person skilled in the art to make and use embodiments of the system. Any portion of the structures and/or principles included in some embodiments can be applied to any and/or all embodiments: it is understood that features from some embodiments presented herein are combinable with other features according to some other embodiments. Thus, some embodiments of the system are not intended to be limited to what is illustrated but are to be accorded the widest scope consistent with all principles and features disclosed herein.

Some embodiments of the system are presented with specific values and/or setpoints. These values and setpoints are not intended to be limiting and are merely examples of a higher configuration versus a lower configuration and are intended as an aid for those of ordinary skill to make and use the system.

Any text in the drawings is part of the system's disclosure and is understood to be readily incorporable into any description of the metes and bounds of the system. Any functional language in the drawings is a reference to the system being configured to perform the recited function, and structures shown or described in the drawings are to be considered as the system comprising the structures recited therein. Any figure depicting contents of graphical user interface is a disclosure of the system being configured to generate a graphical user interface and display the contents on the graphical user interface. It is understood that defining the metes and bounds of the system using a description of images in the drawing does not need a corresponding text description in the written specification to fall with the scope of the disclosure.

Furthermore, acting as Applicant's own lexicographer, Applicant imparts the explicit meaning and/or disavow of claim scope to the following terms:

Applicant defines any use of “and/or” such as, for example, “A and/or B,” or “at least one of A and/or B” to mean element A alone, element B alone, or elements A and B together. In addition, a recitation of “at least one of A, B, and C,” a recitation of “at least one of A, B, or C,” or a recitation of “at least one of A, B, or C or any combination thereof” are each defined to mean element A alone, element B alone, element C alone, or any combination of elements A, B and C, such as AB, AC, BC, or ABC, for example.

“Substantially” and “approximately” when used in conjunction with a value encompass a difference of 5% or less of the same unit and/or scale of that being measured.

“Simultaneously” as used herein includes lag and/or latency times associated with a conventional and/or proprietary computer, such as processors and/or networks described herein attempting to process multiple types of data at the same time. “Simultaneously” also includes the time it takes for digital signals to transfer from one physical location to another, be it over a wireless and/or wired network, and/or within processor circuitry.

As used herein, “can” or “may” or derivations there of (e.g., the system display can show X) are used for descriptive purposes only and is understood to be synonymous and/or interchangeable with “configured to” (e.g., the computer is configured to execute instructions X) when defining the metes and bounds of the system.

In addition, the term “configured to” means that the limitations recited in the specification and/or the claims must be arranged in such a way to perform the recited function: “configured to” excludes structures in the art that are “capable of” being modified to perform the recited function but the disclosures associated with the art have no explicit teachings to do so. For example, a recitation of a “container configured to receive a fluid from structure X at an upper portion and deliver fluid from a lower portion to structure Y” is limited to systems where structure X, structure Y, and the container are all disclosed as arranged to perform the recited function. The recitation “configured to” excludes elements that may be “capable of” performing the recited function simply by virtue of their construction but associated disclosures (or lack thereof) provide no teachings to make such a modification to meet the functional limitations between all structures recited. Another example is “a computer system configured to or programmed to execute a series of instructions X, Y, and Z.” In this example, the instructions must be present on a non-transitory computer readable medium such that the computer system is “configured to” and/or “programmed to” execute the recited instructions: “configure to” and/or “programmed to” excludes art teaching computer systems with non-transitory computer readable media merely “capable of” having the recited instructions stored thereon but have no teachings of the instructions X, Y, and Z programmed and stored thereon. The recitation “configured to” can also be interpreted as synonymous with operatively connected when used in conjunction with physical structures.

It is understood that the phraseology and terminology used herein is for description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The previous detailed description is to be read with reference to the figures, in which like elements in different figures have like reference numerals. The figures, which are not necessarily to scale, depict some embodiments and are not intended to limit the scope of embodiments of the system. Values depicted in example graphical user interfaces are non-limiting and are merely to aid those of ordinary skill in making and using the invention.

Any of the operations described herein that form part of the invention are useful machine operations. The invention also relates to a device or an apparatus for performing these operations. The apparatus can be specially constructed for the required purpose, such as a special purpose computer. When defined as a special purpose computer, the computer can also perform other processing, program execution or routines that are not part of the special purpose, while still being capable of operating for the special purpose. Alternatively, the operations can be processed by a general-purpose computer selectively activated or configured by one or more computer programs stored in the computer memory, cache, or obtained over a network. When data is obtained over a network the data can be processed by other computers on the network, e.g., a cloud of computing resources.

The embodiments of the invention can also be defined as a machine that transforms data from one state to another state. The data can represent an article, that can be represented as an electronic signal and electronically manipulate data. The transformed data can, in some cases, be visually depicted on a display, representing the physical object that results from the transformation of data. The transformed data can be saved to storage generally, or in particular formats that enable the construction or depiction of a physical and tangible object. In some embodiments, the manipulation can be performed by a processor. In such an example, the processor thus transforms the data from one thing to another. Still further, some embodiments include methods can be processed by one or more machines or processors that can be connected over a network. Each machine can transform data from one state or thing to another, and can also process data, save data to storage, transmit data over a network, display the result, or communicate the result to another machine. Computer-readable storage media, as used herein, refers to physical or tangible storage (as opposed to signals) and includes without limitation volatile and non-volatile, removable, and non-removable storage media implemented in any method or technology for the tangible storage of information such as computer-readable instructions, data structures, program modules or other data.

Although method operations are presented in a specific order according to some embodiments, the execution of those steps do not necessarily occur in the order listed unless explicitly specified. Also, other housekeeping operations can be performed in between operations, operations can be adjusted so that they occur at slightly different times, and/or operations can be distributed in a system which allows the occurrence of the processing operations at various intervals associated with the processing, as long as the processing of the overlay operations are performed in the desired way and result in the desired system output.

We claim:

1. A system for secure transfer of electronic documents comprising:
one or more computers comprising one or more processors and one or more non-transitory computer readable media, the one or more non-transitory computer readable media having instructions stored thereon that when executed cause the one or more computers to:
generate, by the one or more processors, a secure processing platform;
generate, by the one or more processors, a claims module;
initiate, by the one or more processors, a secure data connection between the processing platform and the claims module;
send, by the one or more processors, one or more documents from the claims module to the secure processing platform;
analyze, by the one or more processors, the one or more documents for one or more ICD codes; and
associate, by the one or more processors, the one or more ICD codes with one or more conditions.

2. The system of claim 1,
wherein compensation associated with the one or more conditions are sent via a data encrypted network.

3. The system of claim 1,
the one or more non-transitory computer readable media having instructions stored thereon that when executed cause the one or more computers to:
generate, by the one or more processors, a digital marketplace module; and
send, by the one or more processors, compensation associated with the one or more conditions from the digital marketplace module.

4. The system of claim 1,
wherein the one or more ICD codes are ICD-10 codes.

5. The system of claim 1,
wherein the claims module is configured to use a combination of one or more ICD codes and one or more benefit categories to determine the compensation without human intervention.

6. The system of claim 5,
the claims module is configured to determine if more than one benefit category is covered by analysis of one or more eligible documents.

7. The system of claim 1,
the one or more non-transitory computer readable media having instructions stored thereon that when executed cause the one or more computers to:
generate, by the one or more processors, a member portal module; and
generate, by the one or more processors, an employer portal module;
wherein the secure processing platform is configured to accept one or more member inputs and one or more employer inputs from the member portal module and the employer portal module, respectively.

8. The system of claim 1,
the one or more non-transitory computer readable media having instructions stored thereon that when executed cause the one or more computers to:
generate, by the one or more processors, a recommendation engine; and
send, by the one or more processors, the one or more member inputs and the one or more employer inputs from the secure processing platform to the recommendation engine.

9. The system of claim 8,
wherein the recommendation engine is configured to apply a factor to each of the one or more member inputs and each of the one or more employer inputs.

10. The system of claim 9,
wherein the recommendation engine is configured to sum each factor.

11. The system of claim 1,
the one or more non-transitory computer readable media having instructions stored thereon that when executed cause the one or more computers to:
generate, by the one or more processors, a graphical user interface; and
display, on the graphical user interface, a partner portal;
wherein the partner portal is configured to enable a user to input a quote request.

12. The system of claim 11,
the one or more non-transitory computer readable media having instructions stored thereon that when executed cause the one or more computers to:
generate, by the one or more processors, an underwriting module; and
send, by the one or more processors, the quote request to the underwriting module;
wherein the underwriting module is configured estimate risk; and
wherein if the risk is below a predetermined threshold, the underwriting module is configured to automatically provide a quote proportional to the risk.
